(19) United States
(12) Patent Application Publication
(10) Pub. No.: US 2010/0305120 A1
(43) Pub. Date: Dec. 2, 2010

(54) COMPOUNDS FOR TREATING MUSCULAR DYSTROPHY

(75) Inventors: Graham Michael Wynne, Abingdon (GB); Stephen Paul Wren, Abingdon (GB); Renate Maria Van Well, Abingdon (GB); Daniel Robert Chancellor, Abingdon (GB); Frank Schroer, Abingdon (GB); Francis Xavier Wilson, Abingdon (GB)

Correspondence Address: Jones Day, 222 East 41st Street, New York, NY, 10017-6702, US

(73) Assignee: BIOMARIN IGA LIMITED

(21) Appl. No.: 12/599,963

(22) PCT Filed: Apr. 3, 2009
(86) PCT No.: PCT/EP09/02473

§ 371 Date: Jul. 1, 2010

(30) Foreign Application Priority Data
Apr. 4, 2008 (GB) 0806130.1
Feb. 5, 2009 (GB) 0901794.8

(51) Int. Cl. A61K 31/5377; A61K 31/498; A61K 31/4709; A61K 31/454; A61K 31/4439; A61K 31/433; A61K 31/4245; A61K 31/428; A61K 31/427; A61K 31/423; A61K 31/422; A61K 31/416; C07D 413/10; C07D 231/56; A61P 21/00

(52) U.S. Cl. 514/234.5; 514/249; 514/314; 514/322; 514/338; 514/361; 514/364; 514/367; 514/371; 514/375; 514/378; 514/407; 544/140; 548/356.5

(57) ABSTRACT

Compounds of formula (I): wherein X, L¹, R¹, L², R², R³, and R⁴ are as defined herein, are useful in the treatment or prophylaxis of Duchenne muscular dystrophy, Becker muscular dystrophy, or cachexia.

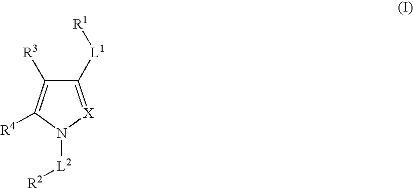

This application claims priority to U.K. Patent Application No. GB0806130.1, filed Apr. 4, 2008, and U.K. Patent Application No. GB0901794.8, filed Feb. 5, 2009; the contents of each of which are incorporated by reference herein in their entireties.

### FIELD

Provided herein are compounds for the treatment of muscular dystrophy and related conditions, including Duchenne muscular dystrophy, compositions comprising the compounds, and methods of use thereof. Also provided herein is a method for the treatment of muscular dystrophy and related conditions, including Duchenne muscular dystrophy.

### BACKGROUND

Duchenne muscular dystrophy (DMD) is a common, genetic neuromuscular disease associated with the progressive deterioration of muscle function, first described over 150 years ago by the French neurologist, Duchenne de Boulogne, after whom the disease is named. DMD has been characterized as an X-linked recessive disorder that affects 1 in 3,500 males caused by mutations in the dystrophin gene. The gene is the largest in the human genome, encompassing 2.6 million base pairs of DNA and containing 79 exons. Approximately 60% of dystrophin mutations are large insertion or deletions that lead to frame-shift errors downstream, whereas approximately 40% are point mutations or small frame-shift rearrangements. The vast majority of DMD patients lack the dystrophin protein. Becker muscular dystrophy is a much milder form of DMD caused by reduction in the amount, or alteration in the size, of the dystrophin protein. The high incidence of DMD (1 in 10,000 sperm or eggs) means that genetic screening will never eliminate the disease, so an effective therapy is highly desirable.

A number of natural and engineered animal models of DMD exist, and provide a mainstay for preclinical studies (Allamand et al., Animal models for muscular dystrophy: valuable tools for the development of therapies. Hum. Mol. Genet. 9, 2459-2467 (2000).) Although the mouse, cat and dog models all have mutations in the DMD gene and exhibit a biochemical dystrophinopathy similar to that seen in humans, they show surprising and considerable variation in terms of their phenotype. Like humans, the canine (golden retriever muscular dystrophy and German short-haired pointer) models have a severe phenotype; these dogs typically die of cardiac failure. Dogs offer the best phenocopy for human disease, and are considered a high benchmark for preclinical studies. Unfortunately, breeding these animals is expensive and difficult, and the clinical time course can be variable among litters.

The mdx mouse is the most widely used model due to availability, short gestation time, time to mature and relatively low cost (Bulfield et al., X chromosome-linked muscular dystrophy (mdx) in the mouse. Proc. Natl. Acad. Sci. USA 81, 1189-1192 (1984).).

Since the discovery of the DMD gene about 20 years ago, varying degrees of success in the treatment of DMD have been achieved in preclinical animal studies, some of which are being followed up in humans. Present therapeutic strategies can be broadly divided into three groups: first, gene therapy approaches; second, cell therapy; and last, pharmacological therapy. Gene- and cell-based therapies offer the fundamental advantage of obviating the need to separately correct secondary defects/pathology (for example, contractures), especially if initiated early in the course of the disease. Unfortunately, these approaches face a number of technical hurdles. Immunological responses against viral vectors, myoblasts and newly synthesized dystrophin have been reported, in addition to toxicity, lack of stable expression and difficulty in delivery.

Pharmacological approaches for the treatment of muscular dystrophy differ from gene- and cell-based approaches in not being designed to deliver either the missing gene and/or protein. In general, the pharmacological strategies use drugs/molecules in an attempt to improve the phenotype by means such as decreasing inflammation, improving calcium homeostasis and increasing muscle progenitor proliferation or commitment. These strategies offer the advantage that they are easy to deliver systemically and can circumvent many of the immunological and/or toxicity issues that are related to vectors and cell-based therapies. Although investigations with corticosteroids and sodium cromoglycate, to reduce inflammation, dantrolene to maintain calcium homeostasis and clenbuterol to increase muscle strength, have produced promising results, none of these potential therapies has yet been shown to be effective in treating DMD.

An alternative pharmacological approach is upregulation therapy. Upregulation therapy is based on increasing the expression of alternative genes to replace a defective gene and is particularly beneficial when an immune response is mounted against a previously absent protein. Upregulation of utrophin, an autosomal paralogue of dystrophin has been proposed as a potential therapy for DMD (Perkins et al., Neuromuscul. Disord., S1: S78-S89 (2002); Khurana et al., Nat. Rev. Drug Discov. 2:379-390 (2003).). When utrophin is over expressed in transgenic mdx mice, it localizes to the sarcolemma of muscle cells and restores the components of the dystrophin-associated protein complex (DAPC), which prevents the dystrophic development and in turn leads to functional improvement of skeletal muscle. Adenoviral delivery of utrophin in the dog has been shown to prevent pathology. Commencement of increased utrophin expression shortly after birth in the mouse model can be effective, and no toxicity is observed when utrophin is ubiquitously expressed, which is promising for the translation of this therapy to humans. Upregulation of endogenous utrophin to sufficient levels to decrease pathology might be achieved by the delivery of small diffusible compounds.

In earlier applications, PCT/GB2007/050055, PCT/GB2007/050056, UK Patent Application No. 0617739.8, UK Patent Application No. 0619282.7, UK Patent Application No. 0623985.9, UK Patent Application No. 0617740.6, UK Patent Application No. 0619283.5, UK Patent Application No. 0714303.5, and UK Patent Application No. 0803906.7, we disclosed compounds which upregulate endogenous utrophin in predictive screens, and thus are useful in the treatment of DMD.

### SUMMARY

Provided herein are compounds for the treatment of muscular dystrophy and related conditions, including DMD, compositions comprising the compounds, and methods of use thereof.

In one embodiment, provided herein are compounds that upregulate endogenous utrophin, and are useful in the treatment of muscular dystrophy, including DMD. In one embodiment, provided herein is a compound of formula (I):

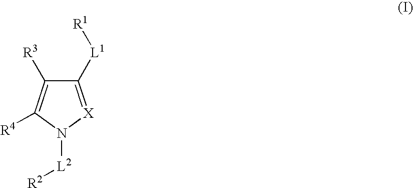

or a tautomer, enantiomer, pharmaceutically acceptable salt, hydrate, solvate, complex or prodrug thereof, wherein X, L¹, L², R¹, R², R³, and R⁴ are defined herein elsewhere, for use in the treatment or prophylaxis of Duchenne muscular dystrophy, Becker muscular dystrophy, or cachexia.

Also provided herein are pharmaceutical compositions comprising a compound of formula (I), including an enantiomer, a mixture of enantiomer, or a mixture of two or more diastereomers; or a pharmaceutically acceptable salt, solvate, hydrate, or prodrug thereof; in combination with one or more pharmaceutically acceptable carriers or excipients.

Also provided herein is a method for the treatment or prophylaxis of Duchenne muscular dystrophy, Becker muscular dystrophy, or cachexia. In one embodiment, the method treats, prevents, or ameliorates one or more symptoms of Duchenne muscular dystrophy, Becker muscular dystrophy, or cachexia. In one embodiment, the method comprises administering to a patient in need thereof an effective amount of a compound of formula (I). In another embodiment, the method comprises administering to a patient in need thereof an effective amount of a pharmaceutically acceptable salt, solvate, hydrate, or prodrug, of a compound of formula (I).

In one embodiment, the compounds of formula (I) are used to treat or prevent Duchenne muscular dystrophy, Becker muscular dystrophy, or cachexia. In another embodiment, the compounds of formula (I) are used in the treatment or prophylaxis of Duchenne muscular dystrophy or Becker muscular dystrophy.

### DETAILED DESCRIPTION

### A. Definitions

Unless defined otherwise, all technical and scientific terms used herein generally have the same meaning as commonly understood by one of ordinary skill in the art to which this disclosure belongs. To facilitate understanding of the disclosure set forth herein, a number of terms are defined below.

As used herein, and unless otherwise specified, the term “C₁-C₆ alkyl” refers to an optionally substituted straight or branched saturated hydrocarbon chain having one to six carbon atoms. In one embodiment, the optional substituent is halo. Examples include, but are not limited to, methyl, ethyl, n-propyl, isopropyl, t-butyl, n-hexyl, trifluoromethyl, and 1,2-dichloroethyl.

As used herein, and unless otherwise specified, the term “C₁-C₄ alkyl” and “C₁-C₁₀ alkyl” have similar meanings except that they contain respectively from one to four and from one to ten carbon atoms.

As used herein, and unless otherwise specified, the term “C₂-C₆ alkenyl” refers to an optionally substituted straight or branched hydrocarbon chain having from two to six carbon atoms, containing at least one carbon-carbon double bond. In one embodiment, the optional substituents is halo. Examples include, but are not limited to, ethenyl, chloroethenyl, 2-propenyl, and 3-hexenyl.

As used herein, and unless otherwise specified, the term “C₂-C₆ alkynyl” refers to an optionally substituted straight or branched hydrocarbon chain having from two to six carbon atoms, containing at least one carbon-carbon triple bond. In one embodiment, the optional substituent is halo. Examples include, but are not limited to, ethynyl, chloroethynyl, 2-propynyl, and 3-hexynyl.

As used herein, and unless otherwise specified, the term “carbocyclic” refers to an optionally substituted ring system in which all the ring atoms are carbon atoms.

As used herein, and unless otherwise specified, the term “heterocyclic” refers to an optionally substituted ring system in which one or more of the ring atoms is a hetero atom selected from N, O and S.

As used herein, and unless otherwise specified, in the carbocyclic and heterocyclic ring systems, one or more ring CH₂ groups may be replaced with a C═O to form a cyclic ketone. In certain embodiment, one or more ring CH₂ groups may be replaced with a C═O to form a cyclic amide.

As used herein, and unless otherwise specified, the term “aromatic” refers to an optionally substituted carbocyclic or heterocyclic ring system which has aromatic character.

In one embodiment, the aromatic ring has one or two rings and from 5 to 10 ring atoms. In bicyclic systems, one of the rings may have aromatic character. Examples of aromatic ring systems include, but are not limited to, phenyl, naphthalene, pyridine, pyrimidine, furan, thiophene, indole, isoindole, benzofuran, benzimidazole, benzimidazoline, benzodioxyl, benzodioxane, quinoline, isoquinoline, tetrahydroisoquinoline, quinazoline, thiazole, benzthiazole, benzoxazole, indazole, and imidazole ring systems.

As used herein, and unless otherwise specified, the term “non-aromatic” refers to an optionally substituted carbocyclic or heterocyclic ring system which may be fully or partially saturated. In one embodiment, the non-aromatic ring is monocyclic and has from 4 to 7 ring atoms. Examples of non-aromatic ring systems include, but are not limited to, cyclopropyl, cyclobutyl, cyclopentyl, cyclohexyl, piperidine, piperazine, morpholine, tetrahydrofuran, and pyrrolidine.

As used herein, and unless otherwise specified, carbocyclic or heterocyclic ring systems are optionally substituted with one or more —X⁴R⁷, wherein:
X⁴ is a bond, —NR⁵—, —S—, —(CR⁵R⁵)_{q}—, —(CR⁵R⁵)_{q}O—, —O(CR⁵R⁵)_{q}—NR⁵C(O)—, —NR⁵C(S)—, —NR⁵C(O)O—, —NR⁵SO₂—, —NR⁵C(O)NR⁵—, —NR⁵C(S)NR⁵—, —NR⁵C(NH)NR⁵—, —NR⁵C(NH)—, —C(O)—, —C(S)—, —C(O)NR⁵—, —C(S)NR⁵—, —SO—, —SO₂—, —SO₂NR⁵—, —OC(O)NR⁵—, or —P(O)OR⁵—;
R⁵ is as defined herein elsewhere;
q is 0, 1, or 2; and
R⁷ is H, C₁-C₆ alkyl, cycloalkyl, heterocyclyl, aryl, or heteroaryl, wherein the alkyl, cycloalkyl, heterocyclyl, aryl, and heteroaryl are each optionally substituted with one or more halo or —O(C₁-C₆ alkyl), and wherein the cycloalkyl, heterocyclyl, aryl, and heteroaryl are each optionally substituted with one or more C₁-C₆ alkyl; or
R⁷ is aralkyl, optionally substituted with one or more halo, —O(C₁-C₆ alkyl), or C₁-C₆ alkyl; or
when X⁴ is a bond, R⁷ may also be halo, NO₂, or CN.

As used herein, and unless otherwise specified, aromatic or non-aromatic ring systems are optionally substituted with one or more —X⁴R⁷, wherein X⁴ and R⁷ are defined herein elsewhere.

As used herein, and unless otherwise specified, the term “cycloalkyl” refers to a cyclic saturated bridged and/or non-bridged monovalent hydrocarbon radical, which may be optionally substituted with one or more —X⁴R⁷, wherein X⁴ and R⁷ are defined herein elsewhere. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may be optionally substituted with one or more —X¹R⁷, wherein X⁴ and R⁷ are defined herein elsewhere.

As used herein, and unless otherwise specified, the term “aralkyl” or “aryl-alkyl” refers to a monovalent alkyl group substituted with aryl. In certain embodiments, the alkyl and aryl moieties are optionally substituted with one or more substituents.

As used herein, and unless otherwise specified, the term “heteroaryl” refers to a monocyclic aromatic group and/or multicyclic aromatic group that contain at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted with one or more —X⁴R⁷, wherein X⁴ and R⁷ are defined herein elsewhere.

As used herein, and unless otherwise specified, the term “heterocyclyl” or “heterocyclic” refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may also be optionally substituted with one or more —X⁴R⁷, wherein X⁴ and R⁷ are defined herein elsewhere.

As used herein, and unless otherwise specified, the term “halo” refers to fluoro, chloro, bromo, or iodo.

As used herein, and unless otherwise specified, for the linker groups L¹ and L², defined herein elsewhere, the right hand side of the L¹ linker group as written is joined to the R¹ moiety and the right hand side of the L² linker group as written is joined to the R² moiety.

As used herein, and unless otherwise specified, appropriate pharmaceutically and veterinarily acceptable salts include basic addition salts such as sodium, potassium, calcium, aluminum, zinc, magnesium, and other metal salts, as well as choline, diethanolamine, ethanolamine, ethyl diamine, and other known basic addition salts.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids.

Salts which are not pharmaceutically or veterinarily acceptable may still be valuable as intermediates.

As used herein, and unless otherwise specified, the term “prodrug” refers to any covalently bonded compounds which release the active parent drug according to formula (I) in vivo.

If a chiral center or another form of isomeric center is present in a compound provided herein, all forms of such isomer or isomers, including enantiomers and diastereoisomers, are intended to be within the scope of this disclosure. Compounds provided herein containing a chiral center may be used as a racemic mixture, an enantiomerically enriched mixture, or the racemic mixture may be separated using known techniques and an individual enantiomer may be used alone.

As used herein, and unless otherwise specified, the term “subject” refers to an animal, including but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

As used herein, and unless otherwise specified, the terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself As used herein, and unless otherwise specified, the terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

As used herein, and unless otherwise specified, the term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington. The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, Fla., 2009.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, and unless otherwise specified, the terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

As used herein, and unless otherwise specified, the terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

As used herein, and unless otherwise specified, the term “solvate” refers to a compound provided herein or a salt thereof, which further includes a stoichiometric or non-stoichiometric amount of solvent bound by non-covalent intermolecular forces. Where the solvent is water, the solvate is a hydrate.

### B. Compounds

Provided herein is a compound of formula (I):

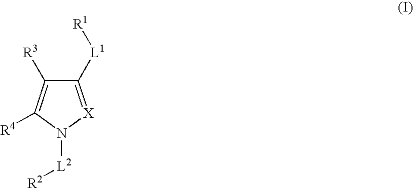

or tautomer, enantiomer, pharmaceutically acceptable salt, hydrate, solvate, complex, or prodrug thereof, wherein
X is CR^{x} or N;
R^{x} is H or C₁-C₆ alkyl;
L¹ is a bond, —(CR⁵R⁵)ₙ—, —NR⁵—, —O—, —S—, —(CR⁵R⁵)ₙNR⁵—, —C(O)NR⁵—, —C(O)NR⁵⁰—, —C(S)NR⁵—, —NR⁵C(O)—, —NR⁵C(S)—, —NR⁵C(NH)—, —SO₂NR⁵—, —NR⁵SO₂—, —C(O)—, —C(S)—, —SO—, —SO₂—, —CR⁵═CR⁵—, —C≡C—, —NR⁵C(O)NR⁵—, —NR⁵C(S)NR⁵—, —NR⁵C(NH)NR⁵—, —NR⁵C(O)O—, or —OC(O)NR⁵—;
R¹ is C₁-C₁₀ alkyl, C₂-C₆ alkenyl, or C₂-C₆ alkynyl, each of which is optionally substituted with one or more OR⁵, N(R⁵)₂, R⁶, or OR⁶; or
R¹ is an optionally substituted 5-10 membered mono- or bicyclic aromatic or 4-7 membered non-aromatic carbocyclic or heterocyclic ring system;
L² is —(CR⁵R⁵)ₙ—, —(CR⁵R⁵)ₙO—, —C(O)—, —C(NR⁵)—, —SO₂—, —C(O)NR⁵—, or —SO₂NR⁵—;
R² is C₁-C₁₀ alkyl, C₂-C₆ alkenyl, or C₂-C₆ alkynyl, each of which is optionally substituted with optionally substituted 5-10 membered mono- or bicyclic aromatic or 4-7 membered non-aromatic carbocyclic or heterocyclic ring system; or
R² is an optionally substituted 5-10 membered mono- or bicyclic aromatic or 4-7 membered non-aromatic carbocyclic or heterocyclic ring system;
Each of R³ and R⁴ is independently hydrogen, C₁-C₆ alkyl, OH, —O(C₁-C₆ alkyl), halo, SOₘR⁵, or NR⁵R⁵; or
R³ and R⁴ together with the carbon atoms to which they are attached form a 5-6 membered aromatic or 5-7 membered non-aromatic carbocyclic or heterocyclic ring system, each of which is optionally substituted with one or more C₁-C₆ alkyl, OH, —O (C₁-C₆ alkyl), halo, SOₘR⁵, C(O)R⁵, or NR⁵R⁵;
m is 0, 1, or 2;
n is 1 or 2;
each R⁵ is independently H or C₁-C₆ alkyl optionally substituted with one or more halo; and
R⁶ is an optionally substituted 5-10 membered mono- or bicyclic aromatic or 4-7 membered non-aromatic carbocyclic or heterocyclic ring system;
   for use in the treatment or prophylaxis of Duchenne muscular dystrophy, Becker muscular dystrophy, or cachexia.

In certain embodiments, provided herein is a compound of formula (I), or a tautomer, enantiomer, pharmaceutically acceptable salt, hydrate, solvate, complex, or prodrug thereof.

In one embodiment, L¹ is a bond, —(CR⁵R⁵)ₙ—, —NR⁵—, —O—, —S—, —(CR⁵R⁵)ₙNR⁵—, —C(O)NR⁵—, —C(S)NR⁵—, —NR⁵C(O)—, —NR⁵C(S)—, —NR⁵C(NH)—, —SO₂NR⁵—, —NR⁵SO₂—, —C(S)—, —SO—, —SO₂—, —CR⁵═CR⁵—, —NR⁵C(O)NR⁵—, —NR⁵C(S)NR⁵—, —NR⁵C(NH)NR⁵—, —NR⁵C(O)O—, or —OC(O)NR⁵—; wherein R⁵ and n are defined herein elsewhere.

In one embodiment, L¹ is a bond, —CH₂—, —CH₂CH₂—, —C(O)NR⁵—, —C(S)NR⁵—, —NR⁵—C(O)—NR⁵—, —NR⁵C(O)—, —C(O)—, —NR⁵—SO₂—, —C(O)NR⁵—O—, —S—, —SO₂—, or —CH₂NR⁵—; wherein R⁵ is H or C₁-C₄ alkyl.

In another embodiment, L¹ is a bond, —CH₂—, —C(O)NH—, —C(O)NCH₃—, —C(S)NH—, —C(S)NCH₃—, —NHC(O)NH—, —NHC(O)—, —CO—, —NHSO₂—, —C(O)NH—O—, or —CH₂NH—.

In another embodiment, L¹ is a bond, —C(O)NH—, —NHC(O)—, —NHC(O)NH—, or —CH₂NH—.

In one embodiment, L¹ is a bond. In another embodiment, L¹ is —(CR⁵R⁵)ₙ—. In another embodiment, L¹ is —NR⁵—. In another embodiment, L¹ is —O—. In another embodiment, L¹ is —S—. In another embodiment, L¹ is —(CR⁵R⁵)ₙNR⁵—. In another embodiment, L¹ is —C(O)NR⁵—. In another embodiment, L¹ is —C(O)NR⁵⁰—. In another embodiment, L¹ is —C(S)NR⁵—. In another embodiment, L¹ is —NR⁵C(O)—. In another embodiment, L¹ is —NR⁵C(S)—. In another embodiment, L¹ is —NR⁵C(NH)—. In another embodiment, L¹ is —SO₂NR⁵—. In another embodiment, L¹ is —NR⁵SO₂—. In another embodiment, L¹ is —C(O)—. In another embodiment, L¹ is —C(S)—. In another embodiment, L¹ is —SO—. In another embodiment, L¹ is —SO₂—. In another embodiment, L¹ is —CR⁵═CR⁵—. In another embodiment, L¹ is —C≡C—. In another embodiment, L¹ is —NR⁵C(O)NR⁵—. In another embodiment, L¹ is —NR⁵C(S)NR⁵—. In another embodiment, L¹ is —NR⁵C(NH)NR⁵—. In another embodiment, L¹ is —NR⁵C(O)O—. In another embodiment, L¹ is —OC(O)NR⁵—. In another embodiment, L¹ is —CH₂—. In another embodiment, L¹ is —CH₂CH₂—. In another embodiment, L¹ is —CH₂NR⁵—. In another embodiment, L¹ is —C(O)NH—. In another embodiment, L¹ is —C(O)NCH₃—. In another embodiment, L¹ is —C(S)NH—. In another embodiment, L¹ is —C(S)NCH₃—. In another embodiment, L¹ is —NHC(O)NH—. In another embodiment, L¹ is —NHC(O)—. In another embodiment, L¹ is —NHSO₂—. In another embodiment, L¹ is —C(O)NH—O—. In another embodiment, L¹ is —CH₂NH—. R⁵ and n are defined herein elsewhere.

In one embodiment, R¹ is C₁-C₁₀ alkyl, C₂-C₆ alkenyl, or C₂-C₆ alkynyl, any of which is optionally substituted with one or more OR⁵, R⁶, or OR⁶. R⁵ and R⁶ are defined herein elsewhere.

In another embodiment, R¹ is a 5-10 membered mono- or bicyclic aromatic or 4-7 membered non-aromatic carbocyclic or heterocyclic ring system, each of which is unsubstituted or substituted with one or more R⁵, R⁶, —C(O)NR⁵R⁵, —C(O)NHR⁶, —NR⁵C(O)R⁵, —NR⁵C(O)R⁶, —C(O)OR⁵, —C(O)OR⁶, —C(O)R⁵, —C(O)R⁶, —(CH₂)_{q}OR⁵, —(CH₂)_{q}OR⁶, —SO₂R⁵, —SO₂R⁶, halo, or CN. R⁵, R⁶, and q are defined herein elsewhere.

In another embodiment, R¹ is a 5-10 membered mono- or bicyclic aromatic or 4-7 membered non-aromatic carbocyclic or heterocyclic ring system, each of which is unsubstituted or substituted with one or more R⁵, R⁶, —C(O)NR⁵R⁵, —C(O)NHR⁶, —C(O)OR⁵, —C(O)OR⁶, —C(O)R⁵, —C(O)R⁶, —(CH₂)_{q}OR⁵, —(CH₂)_{q}OR⁶, —SO₂R⁵, —SO₂R⁶, halo, or CN. R⁵, R⁶, and q are defined herein elsewhere.

In another embodiment, R¹ is optionally substituted phenyl, pyridine, pyrimidine, pyridazine, pyrrole, furan, thiophene, benzofuran, benzothiazole, benzodioxolane, benzodioxyl, benzodioxane, thiadiazole, isoxazole, cyclopropyl, cyclobutyl, piperazine, pyrrolidine, pyrrolidinone, piperidine, piperazine, morpholine, thiazole, naphthalene, quinoxaline, quinoline, benzoxazole, indane, or tetrahydronaphthalene. Optional substituents of the ring systems are defined herein elsewhere.

In another embodiment, R¹ is optionally substituted phenyl, pyridine, pyrimidine, pyridazine, pyrrole, furan, thiophene, benzofuran, benzothiazole, benzodioxolane, benzodioxyl, benzodioxane, thiadiazole, isoxazole, cyclopropyl, cyclobutyl, piperazine, pyrrolidine, pyrrolidinone, piperidine, piperazine, or morpholine. Optional substituents of the ring systems are defined herein elsewhere.

In another embodiment, R¹ is optionally substituted phenyl, pyridine, pyrimidine, pyridazine, pyrrole, furan, thiophene, benzofuran, benzothiazole, benzodioxolane, thiadiazole, isoxazole, cyclopropyl, cyclobutyl, piperazine, pyrrolidine, pyrrolidinone, piperidine, piperazine, or morpholine. Optional substituents of the ring systems are defined herein elsewhere.

In another embodiment, R¹ is a 5-10 membered mono- or bicyclic aromatic or 4-7 membered non-aromatic carbocyclic or heterocyclic ring system, each of which is optionally substituted with one or more CONH₂, CON(CH₃)₂, CONH(C₁-C₃ alkyl), CO(C₁-C₃ alkyl), C(O)heterocyclyl, COOH, COO(C₁-C₃ alkyl), SO₂CH₃, CH₂CH₂OH, O(C₁-C₃ alkyl), NHC(O)(C₁-C₃ alkyl), heterocyclyl, phenoxy, C₁-C₃ alkyl, (CH₂)_{q}OH, (CH₂)_{q}O-phenyl, cyano, and halo; wherein q is defined herein elsewhere. C₁-C₃ alkyl include, but are not limited to, methyl, ethyl, propyl, trifluororomethyl, difluoromethyl, and isopropyl. In certain embodiment, C₁-C₃ alkyl include, but are not limited to, methyl, ethyl, trifluororomethyl, difluoromethyl, and isopropyl.

In another embodiment, R¹ is a 5-10 membered mono- or bicyclic aromatic or 4-7 membered non-aromatic carbocyclic or heterocyclic ring system, each of which is optionally substituted with one or more CONH₂, CON(CH₃)₂, CONH(C₁-C₃ alkyl), CO(C₁-C₃ alkyl), C(O)heterocyclyl, COOH, COO(C₁-C₃ alkyl), SO₂CH₃, CH₂CH₂OH, O(C₁-C₃ alkyl), phenoxy, C₁-C₃ alkyl, (CH₂)_{q}OH, (CH₂)_{q}O-phenyl, cyano, and halo; wherein q is defined herein elsewhere. C₁-C₃ alkyl include, but are not limited to, methyl, ethyl, propyl, trifluororomethyl, difluoromethyl, and isopropyl. In certain embodiment, C₁-C₃ alkyl include, but are not limited to, methyl, ethyl, trifluororomethyl, difluoromethyl, and isopropyl.

In one embodiment, R¹ is C₁-C₁₀ alkyl, optionally substituted with OR⁵, N(R⁵)₂, R⁶, or OR⁶. In one embodiment, R¹ is C₁-C₁₀ alkyl, optionally substituted with OR⁵, R⁶, or OR⁶. In another embodiment, R¹ is —(CH₂)—R⁶ or —(CH₂)₂—R⁶, wherein R⁶ is phenyl, furan, or tetrahydrofuran, each of which is optionally substituted with one or more C₁-C₃ alkyl, O(C₁-C₃ alkyl), or halo.

In one embodiment, R¹ is C₁-C₁₀ alkyl optionally substituted with one or more OR⁵, N(R⁵)₂, R⁶, or OR⁶. In another embodiment, R¹ is C₂-C₆ alkenyl optionally substituted with one or more OR⁵, N(R⁵)₂, R⁶, or OR⁶. In another embodiment, R¹ is C₂-C₆ alkynyl optionally substituted with one or more OR⁵, N(R⁵)₂, R⁶, or OR⁶. In another embodiment, R¹ is C₁-C₁₀ alkyl optionally substituted with one or more OR⁵, R⁶, or OR⁶. In another embodiment, R¹ is C₂-C₆ alkenyl optionally substituted with one or more OR⁵, R⁶, or OR⁶. In another embodiment, R¹ is C₂-C₆ alkynyl optionally substituted with one or more OR⁵, R⁶, or OR⁶.

In one embodiment, R¹ is optionally substituted monocyclic aromatic. In another embodiment, R¹ is optionally substituted bicyclic aromatic. In another embodiment, R¹ is optionally substituted 4-7 membered non-aromatic carbocyclic ring system. In another embodiment, R¹ is optionally substituted 4-7 membered non-aromatic heterocyclic ring system. Optional substituents of the ring systems are defined herein elsewhere.

In one embodiment, R¹ is optionally substituted phenyl. In another embodiment, R¹ is optionally substituted pyridine. In another embodiment, R¹ is optionally substituted pyrimidine. In another embodiment, R¹ is optionally substituted pyridazine. In another embodiment, R¹ is optionally substituted pyrrole. In another embodiment, R¹ is optionally substituted furan. In another embodiment, R¹ is optionally substituted thiophene. In another embodiment, R¹ is optionally substituted benzofuran. In another embodiment, R¹ is optionally substituted benzothiazole. In another embodiment, R¹ is optionally substituted benzodioxolane. In another embodiment, R¹ is optionally substituted benzodioxyl. In another embodiment, R¹ is optionally substituted benzodioxane. In another embodiment, R¹ is optionally substituted thiadiazole. In another embodiment, R¹ is optionally substituted isoxazole. In another embodiment, R¹ is optionally substituted cyclopropyl. In another embodiment, R¹ is optionally substituted cyclobutyl. In another embodiment, R¹ is optionally substituted piperazine. In another embodiment, R¹ is optionally substituted pyrrolidine. In another embodiment, R¹ is optionally substituted pyrrolidinone. In another embodiment, R¹ is optionally substituted piperidine. In another embodiment, R¹ is optionally substituted piperazine. In another embodiment, R¹ is optionally substituted morpholine. In another embodiment, R¹ is optionally substituted thiazole. In another embodiment, R¹ is optionally substituted naphthalene. In another embodiment, R¹ is optionally substituted quinoxaline. In another embodiment, R¹ is optionally substituted quinoline. In another embodiment, R¹ is optionally substituted benzoxazole. In another embodiment, R¹ is optionally substituted indane. In another embodiment, R¹ is optionally substituted tetrahydronaphthalene. Optional substituents of the ring systems are defined herein elsewhere.

In one embodiment, R¹ is:

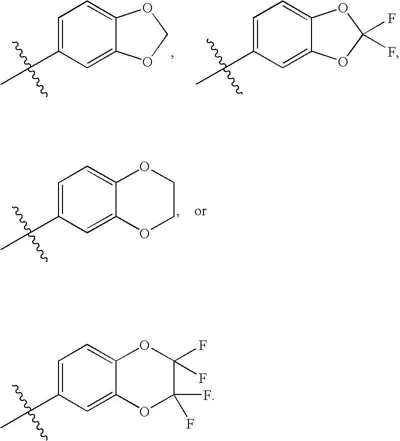

In one embodiment, L² is —(CH₂)ᵣ—, —(CH₂)ᵣO—, —C(O)—, or SO₂—; wherein r is 1, 2 or 3. In another embodiment, L² is —CH₂—, —(CH₂)₂—, or —C(O)—. In another embodiment, L² is —(CH₂)O— or —(CH₂)₂O—.

In one embodiment, L² is —(CR⁵R⁵)ₙ—. In another embodiment, L² is —(CR⁵R⁵)ₙO—. In another embodiment, L² is —C(O)—. In another embodiment, L² is —C(NR⁵)—. In another embodiment, L² is —SO₂—. In another embodiment, L² is —C(O)NR⁵—. In another embodiment, L² is —SO₂NR⁵—. In another embodiment, L² is —(CH₂)ᵣ—. In another embodiment, L² is —(CH₂)ᵣO—. In another embodiment, L² is CH₂—. In another embodiment, L² is —(CH₂)₂—. In another embodiment, L² is —(CH₂)O—. In another embodiment, L² is —(CH₂)₂O—. R⁵, n, and r are defined herein elsewhere.

In one embodiment, R² is optionally substituted 5- or 6-membered aromatic or non-aromatic cyclic groups, including but not limited to, phenyl, pyridine, piperidine, cyclohexyl, pyrimidine. In another embodiment, R² is optionally substituted thiophene, isoxazole, or oxadiazole. In another embodiment, R² is optionally substituted naphthalene or benzodioxolane. Optional substituents of the ring systems are defined herein elsewhere.

In one embodiment, R² is optionally substituted phenyl. In another embodiment, R² is optionally substituted pyridine. In another embodiment, R² is optionally substituted piperidine. In another embodiment, R² is optionally substituted cyclohexyl. In another embodiment, R² is optionally substituted pyrimidine. In another embodiment, R² is optionally substituted thiophene. In another embodiment, R² is optionally substituted isoxazole. In another embodiment, R² is optionally substituted oxadiazole. In another embodiment, R² is optionally substituted naphthalene. In another embodiment, R² is optionally substituted benzodioxolane. Optional substituents of the ring systems are defined herein elsewhere.

In one embodiment, R² is optionally substituted with one or more halo, cyano, R⁷, C(O)NR⁵R⁷, —C(O)R⁷, —SO₂NR⁵R⁷, —SO₂N(R⁵)(OR⁷), —(CR⁵R⁵)_{q}R⁷, —SO₂R⁷, —(CR⁵R⁵)_{q}OR⁷, or —O(CR⁵R⁵)_{q}R⁷; wherein R⁵, R⁷, and q are defined herein elsewhere; and when R⁵ and R⁷ are attached to the same nitrogen atom, R⁵ and R⁷ may be combined with that nitrogen atom to form a 4 to 7 membered saturated or unsaturated ring system which may contain one or more additional heteroatoms.

In one embodiment, R² is optionally substituted with one or more halo, cyano, R⁷, C(O)NR⁵R⁷, —C(O)R⁷, —SO₂NR⁵R⁷, —(CR⁵R⁵)_{q}R⁷, —SO₂R⁷, —(CR⁵R⁵)_{q}OR⁷, or —O(CR⁵R⁵)_{q}R⁷; wherein R⁵, R⁷, and q are defined herein elsewhere; and when R⁵ and R⁷ are attached to the same nitrogen atom, R⁵ and R⁷ may be combined with that nitrogen atom to form a 4 to 7 membered saturated or unsaturated ring system which may contain one or more additional heteroatoms.

In one embodiment, R⁵ is C₁-C₄ alkyl, including but not limited to, methyl, ethyl, trifluoromethyl, and t-butyl. In another embodiment, R⁵ is H. In another embodiment, R⁵ is methyl. In another embodiment, R⁵ is ethyl. In another embodiment, R⁵ is trifluoromethyl. In another embodiment, R⁵ is t-butyl. In another embodiment, R⁵ is propyl. In another embodiment, R⁵ is isopropyl.

In one embodiment, R⁷ is C₁-C₄ alkyl, C₁-C₄ alkenyl, or C₁-C₄ alkynyl, including but are not limited to, methyl, ethyl, trifluoromethyl, t-butyl, 2-propenyl and 2-propynyl. In another embodiment, R⁷ is C₁-C₄ alkyl. In another embodiment, R⁷ is trifluoromethyl. In another embodiment, R⁷ is methyl. In another embodiment, R⁷ is ethyl. In another embodiment, R⁷ is t-butyl. In another embodiment, R⁷ is 2-propenyl. In another embodiment, R⁷ is 2-propynyl. In another embodiment, R⁷ is propyl. In another embodiment, R⁷ is isopropyl. In another embodiment, R⁷ is phenyl. In another embodiment, R⁷ is benzodioxolane. In another embodiment, R⁷ is pyrrolidine. In another embodiment, R⁷ is morpholine. In another embodiment, R⁷ is piperidine. In another embodiment, R⁷ is pyrrolidinedione.

In one embodiment, X is N. In another embodiment, X is CR^{x}.

In one embodiment, R³ and R⁴ combine to form a fused ring system. In one embodiment, R³ and R⁴ combine to form an optionally substituted benzene ring. In another embodiment, R³ and R⁴ combine to form an optionally substituted cyclohexenyl or cyclopentenyl ring. In another embodiment, R³ and R⁴ are each hydrogen or C₁-C₄ alkyl.

In one embodiment, R³ and R⁴ combine to form an optionally substituted benzene ring and X is N; and the compounds of formula (I) are indazole derivatives. In another embodiment, R³ and R⁴ combine to form an optionally substituted benzene ring and X is CR^{x}; and the compounds of formula (I) are indole derivatives.

In one embodiment, provided herein is a compound of formula (Ia):

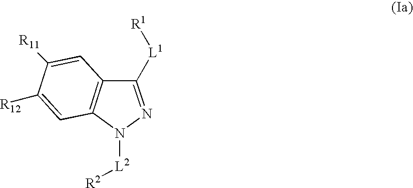

or a tautomer, enantiomer or pharmaceutically acceptable salt, hydrate, solvate, complex or prodrug thereof, wherein

R¹¹ and R¹² are each independently H, C₁-C₆ alkyl, —O(C₁-C₆ alkyl), or halo; and L¹, L², R¹, and R² are defined herein elsewhere.

In one embodiment, R¹¹ is H. In another embodiment, R¹¹ is C₁-C₆ alkyl, including but not limited to, methyl and CF₃. In another embodiment, R¹¹ is —O(C₁-C₆ alkyl). In another embodiment, R¹¹ is halo.

In one embodiment, R¹² is H. In another embodiment, R¹² is C₁-C₆ alkyl, including but not limited to, methyl and CF₃. In another embodiment, R¹² is —O(C₁-C₆ alkyl). In another embodiment, R¹² is halo.

In one embodiment, R¹¹ is H and R¹² is H. In another embodiment, R¹¹ is H and R¹² is F. In another embodiment, R¹² is H and R¹¹ is F. In another embodiment, R¹² is H and R¹¹ is CF₃. In another embodiment, R¹² is H and R¹¹ is OMe. In another embodiment, R¹² is H and R¹¹ is methyl.

Any combinations of R¹, R², R³, R⁴, R⁵, R⁶, R⁷, R¹¹, R¹², R^{x}, X, X⁴, L¹, L², m, n, q, and r are encompassed by this disclosure and specifically provided herein.

In one embodiment, specific examples of compounds of formula (I) include, but are not limited to, the following:
1. N-(benzo[d]thiazol-2-yl)-1-(4-(N-methoxy-N-methylsulfamoyl)benzyl)-1H-indazole-3-carboxamide;

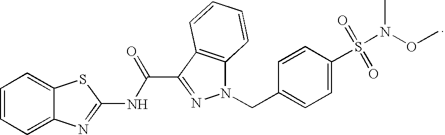

2. 1-(1-(4-((benzo[d][1,3]dioxol-5-yloxy)methyl)benzyl)-5-(trifluoromethyl)-1H-indazol-3-yl)-3-(2,5-dimethoxyphenyl)urea;

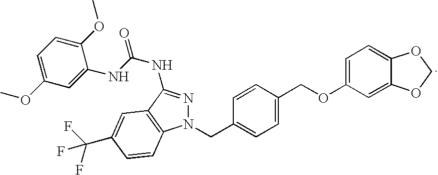

3. 1-(4-(methylsulfonyl)benzyl)-N-(thiazol-2-yl)-1H-indazole-3-carboxamide;

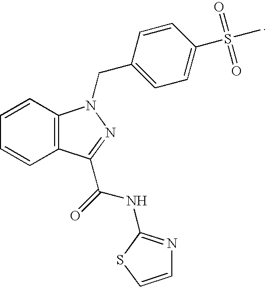

4. N-(benzo[d][1,3]dioxol-5-yl)-1-(4-(methylsulfonyl)benzyl)-1H-indazole-3-carboxamide;

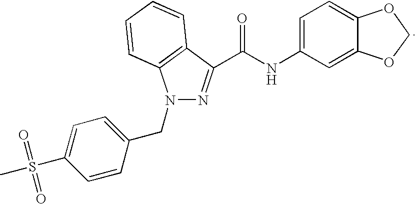

5. N-(4-methoxyphenyl)-1-(3-phenoxypropyl)-1H-indazole-3-carboxamide;

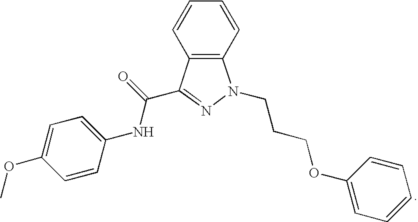

6. 1-(4-(N-methyl-N-(prop-2-ynyl)sulfamoyl)benzyl)-N-(4-(piperidin-1-yl)phenyl)-1H-indazole-3-carboxamide;

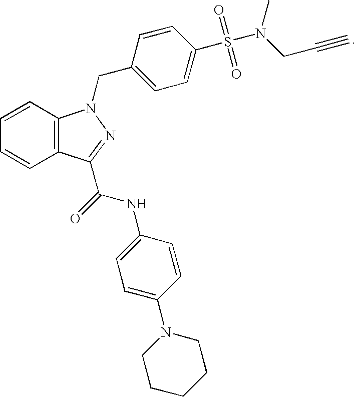

7. N-(benzo[d]thiazol-2-yl)-1-(4-(N,N-diethylsulfamoyl)benzyl)-1H-indazole-3-carboxamide;

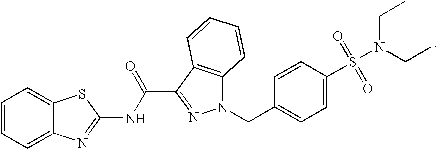

8. 5-methoxy-1-(4-(N-methoxy-N-methylsulfamoyl)benzyl)-N-(4-(piperidin-1-yl)phenyl)-1H-indazole-3-carboxamide;

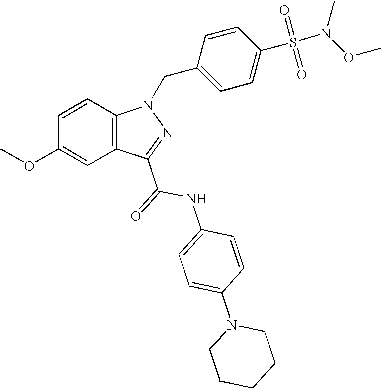

9. 1-(1-(4-((benzo[d][1,3]dioxol-5-yloxy)methyl)benzyl)-5-(trifluoromethyl)-1H-indazol-3-yl)-3-(3-fluorophenyl)urea;

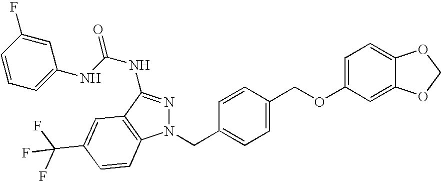

10. 1-(4-(N,N-diethylsulfamoyl)benzyl)-N-(4-(piperidin-1-yl)phenyl)-1H-indazole-3-carboxamide;

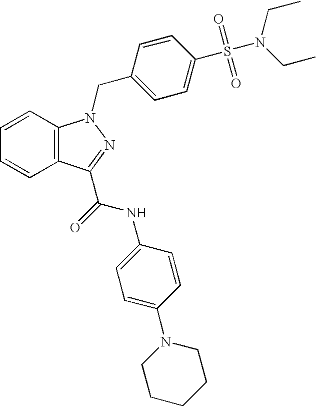

11. methyl 4-(3-(1-(4-((3-methoxyphenoxy)methyl)benzyl)-1H-indazol-3-yl)ureido)benzoate;

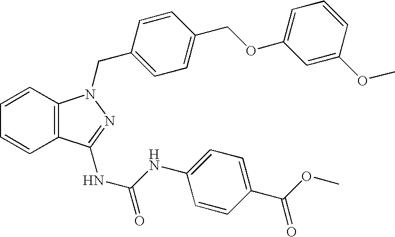

12. 1-(2,6-dimethoxyphenyl)-3-(1-(4-(trifluoromethyl)benzyl)-1H-indazol-3-yl)urea;

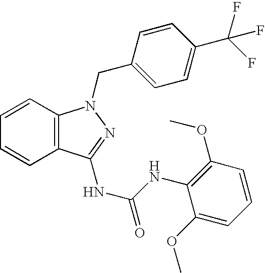

13. 1-(1-((6-chlorobenzo[d][1,3]dioxol-5-yl)methyl)-5-methyl-1H-indazol-3-yl)-3-(2-isopropylphenyl)urea;

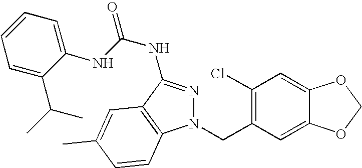

14. 1-(3-phenoxypropyl)-N-(4-(piperidin-1-yl)phenyl)-1H-indazole-3-carboxamide;

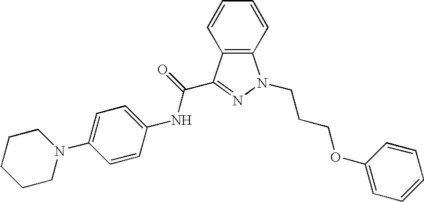

15. 1-m-tolyl-3-(1-(4-(trifluoromethyl)benzyl)-1H-indazol-3-yl)urea;

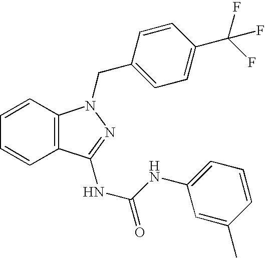

16. 1-(3-phenoxypropyl)-N-(thiazol-2-yl)-1H-indazole-3-carboxamide;

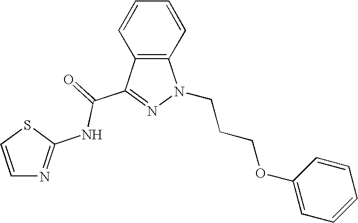

17. N-(benzo[d]thiazol-2-yl)-1-(4-(morpholine-4-carbonyl)benzyl)-1H-indazole-3-carboxamide;

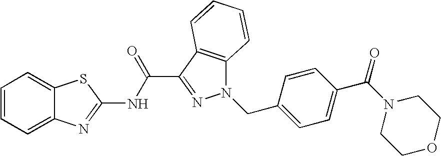

18. 1-(4-(methylsulfonyl)benzyl)-N-(4-(piperidin-1-yl)phenyl)-1H-indazole-3-carboxamide;

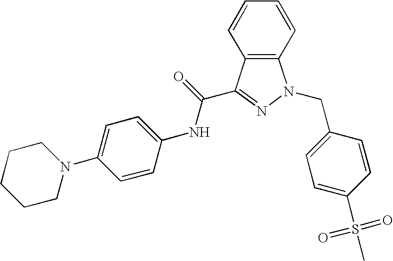

19. N-(4-isopropylphenyl)-1-(4-(morpholine-4-carbonyl)benzyl)-1H-indazole-3-carboxamide;

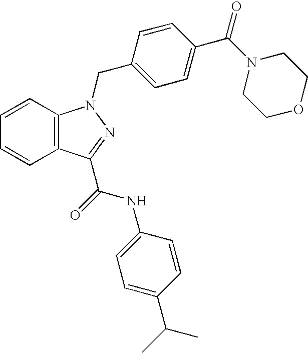

20. 1-(4-(benzyl(methyl)carbamoyl)benzyl)-N-(4-(trifluoromethyl)phenyl)-1H-indazole-3-carboxamide;

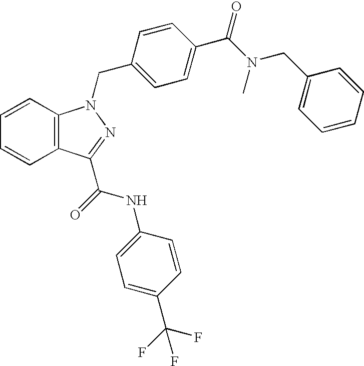

21. N-(benzo[d][1,3]dioxol-5-yl)-1-(4-(N-methyl-N-(prop-2-ynyl)sulfamoyl)benzyl)-1H-indazole-3-carboxamide;

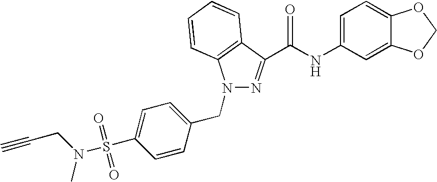

22. 1-(4-(benzyl(methyl)carbamoyl)benzyl)-N-(4-isopropylphenyl)-1H-indazole-3-carboxamide;

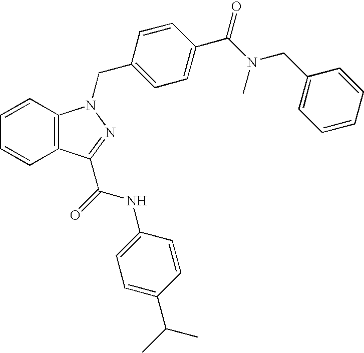

23. 1-(4-(methylsulfonyl)benzyl)-N-(4-(trifluoromethoxy)phenyl)-1H-indazole-3-carboxamide;

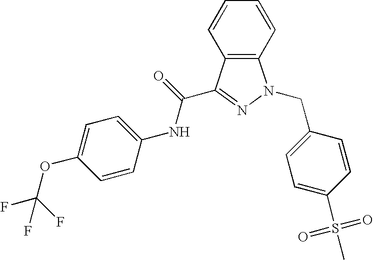

24. N-(benzyloxy)-1-(4-(N-methyl-N-phenylsulfamoyl)benzyl)-1H-indazole-3-carboxamide;

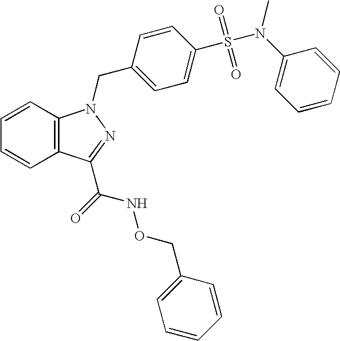

25. N-(2,2-difluorobenzo[d][1,3]dioxol-5-yl)-1-(4-(morpholine-4-carbonyl)benzyl)-1H-indazole-3-carboxamide;

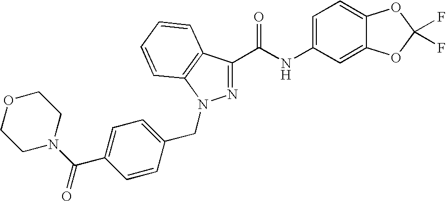

26. N-(4-isopropylphenyl)-1-(2-phenoxyethyl)-1H-indazole-3-carboxamide;

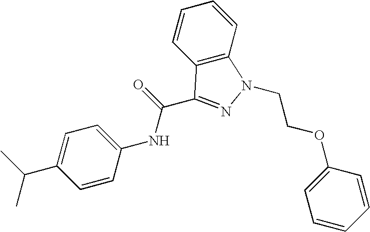

27. 1-(biphenyl-4-ylmethyl)-N-(4-isopropylphenyl)-1H-indazole-3-carboxamide;

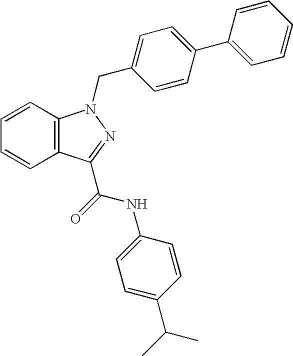

28. 1-(2-fluorobenzyl)-3-(1-(4-((3-methoxyphenoxy)methyl)benzyl)-1H-indazol-3-yl)urea;

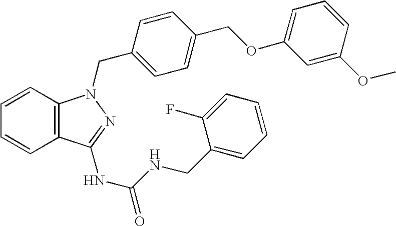

29. 1-(1-(4-((benzo[d][1,3]dioxol-5-yloxy)methyl)benzyl)-5-(trifluoromethyl)-1H-indazol-3-yl)-3-(2-(trifluoromethyl)phenyl)urea;

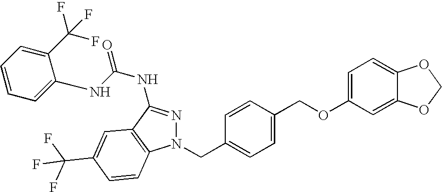

30. 1-(2,3-dihydro-1H-inden-5-yl)-3-(1-(4-((3-methoxyphenoxy)methyl)benzyl)-1H-indazol-3-yl)urea;

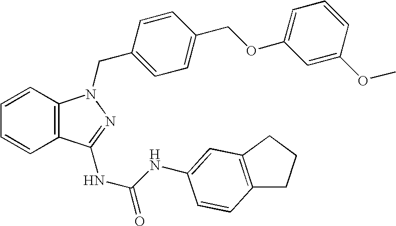

31. 1-(1-(4-((benzo[d][1,3]dioxol-5-yloxy)methyl)benzyl)-5-(trifluoromethyl)-1H-indazol-3-yl)-3-(2,4-dimethoxyphenyl)urea;

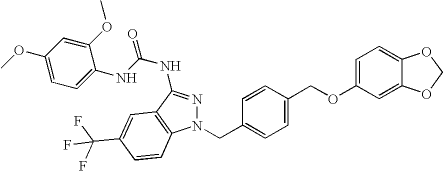

32. 1-(1-((6-chlorobenzo[d][1,3]dioxol-5-yl)methyl)-1H-indazol-3-yl)-3-(3-methylbenzyl)urea;

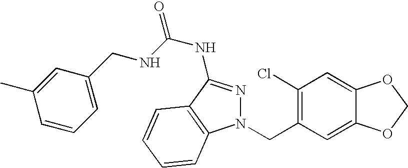

33. N-(1-(4-((2,5-dioxopyrrolidin-1-yl)methyl)benzyl)-1H-indazol-3-yl)quinoxaline-2-carboxamide;

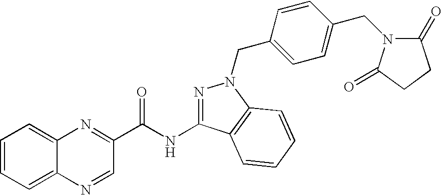

34. N-(4-methoxyphenyl)-1-(4-(methylsulfonyl)benzyl)-1H-indazole-3-carboxamide;

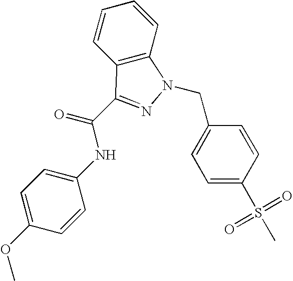

35. N-(2,2-difluorobenzo[d][1,3]dioxol-5-yl)-1-(4-(methylsulfonyl)benzyl)-1H-indazole-3-carboxamide;

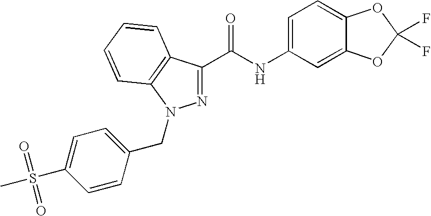

36. 1-(4-(N-methoxy-N-methylsulfamoyl)benzyl)-N-(thiazol-2-yl)-1H-indazole-3-carboxamide;

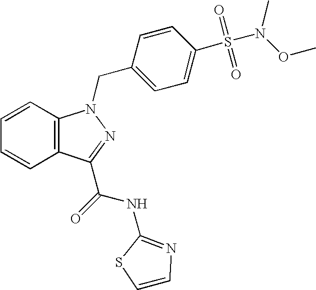

37. 1-(1-((6-chlorobenzo[d][1,3]dioxol-5-yl)methyl)-1H-indazol-3-yl)-3-(4-methoxyphenethyl)urea;

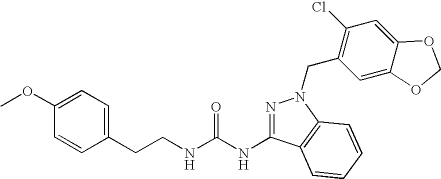

38. N-(4-carbamoylphenyl)-1-(4-(morpholine-4-carbonyl)benzyl)-1H-indazole-3-carboxamide;

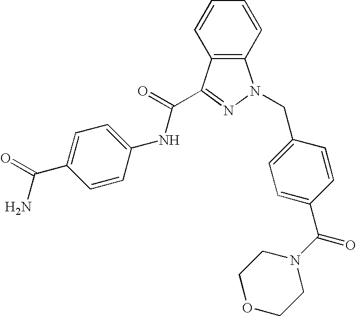

39. 1-(4-(N,N-diethylsulfamoyl)benzyl)-N-(pyridin-2-yl)-1H-indazole-3-carboxamide;

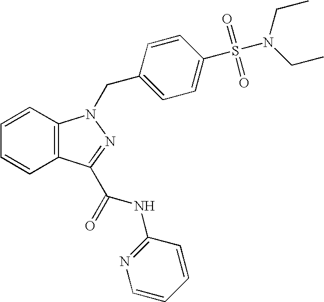

40. 1-(2-(trifluoromethoxy)phenyl)-3-(1-(4-(trifluoromethyl)benzyl)-1H-indazol-3-yl)urea;

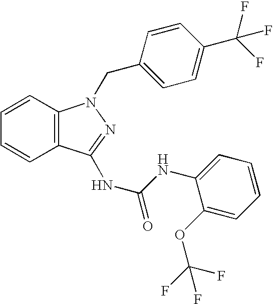

41. 1-(4-(N,N-diethylsulfamoyl)benzyl)-N-(2-methyl-1,3-dioxoisoindolin-5-yl)-1H-indazole-3-carboxamide;

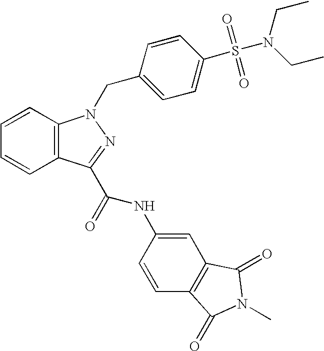

42. N-(benzo[d][1,3]dioxol-5-yl)-1-(4-tert-butylbenzyl)-1H-indazole-3-carboxamide;

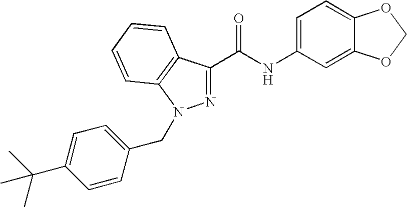

43. 1-(2-(4-fluorophenoxy)ethyl)-N-(4-(piperidin-1-yl)phenyl)-1H-indazole-3-carboxamide;

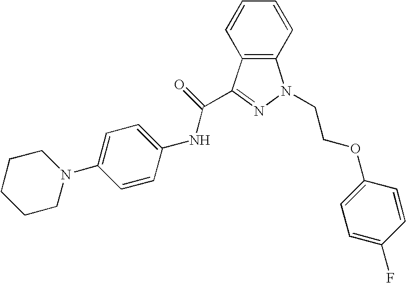

44. 1-(2-methoxybenzyl)-3-(1-(4-((3-methoxyphenoxy)methyl)benzyl)-1H-indazol-3-yl)urea;

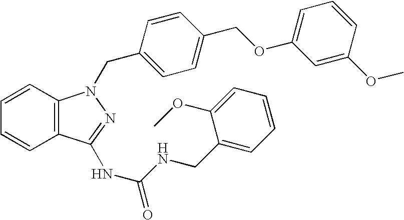

45. N-(benzo[d][1,3]dioxol-5-yl)-1-(3-phenoxypropyl)-1H-indazole-3-carboxamide;

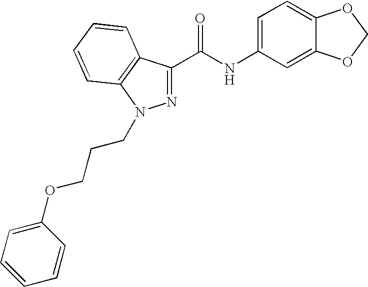

46. N-(benzo[d][1,3]dioxol-5-yl)-1-(2-(4-fluorophenoxy)ethyl)-1H-indazole-3-carboxamide;

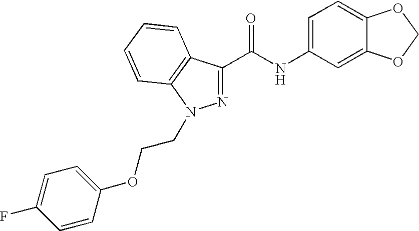

47. 1-(1-((6-chlorobenzo[d][1,3]dioxol-5-yl)methyl)-1H-indazol-3-yl)-3-(2-fluorobenzyl)urea;

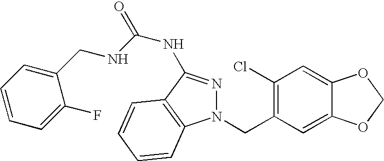

48. 1-(4-tert-butylbenzyl)-N-(2,2-difluorobenzo[d][1,3]dioxol-5-yl)-1H-indazole-3-carboxamide;

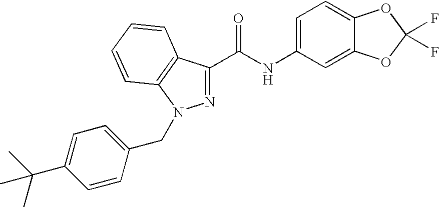

49. 1-(4-(N-methoxy-N-methylsulfamoyl)benzyl)-N-(pyridin-2-yl)-1H-indazole-3-carboxamide;

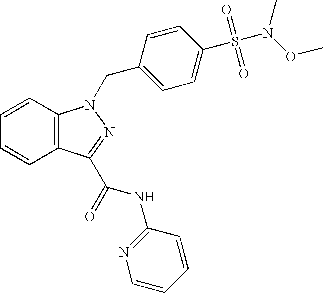

50. N-(benzo[d][1,3]dioxol-5-yl)-1-(biphenyl-4-ylmethyl)-1H-indazole-3-carboxamide;

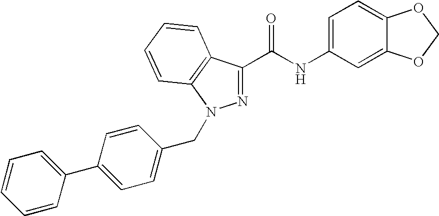

51. 1-(1-(4-((benzo[d][1,3]dioxol-5-yloxy)methyl)benzyl)-5-(trifluoromethyl)-1H-indazol-3-yl)-3-m-tolylurea;

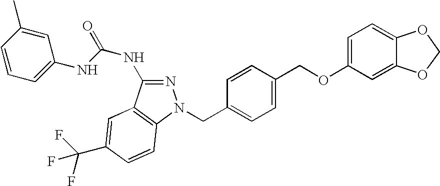

52. 1-(1-((6-chlorobenzo[d][1,3]dioxol-5-yl)methyl)-1H-indazol-3-yl)-3-(3,5-dimethoxyphenyl)urea;

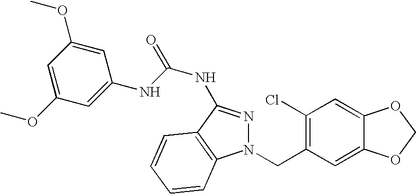

53. 1-(2,3-dihydro-1H-inden-5-yl)-3-(1-(4-(trifluoromethyl)benzyl)-1H-indazol-3-yl)urea;

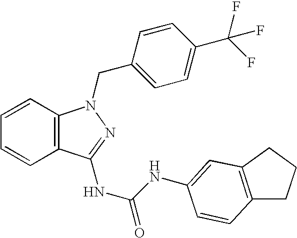

54. 1-(3,4-dimethoxyphenyl)-3-(1-(4-((3-methoxyphenoxy)methyl)benzyl)-1H-indazol-3-yl)urea;

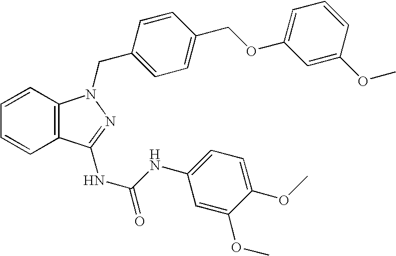

55. 1-(1-((6-chlorobenzo[d][1,3]dioxol-5-yl)methyl)-1H-indazol-3-yl)-3-(furan-2-ylmethyl)urea;

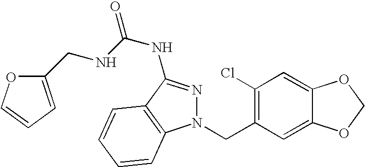

56. N-(4-methoxyphenyl)-1-(2-phenoxyethyl)-1H-indazole-3-carboxamide;

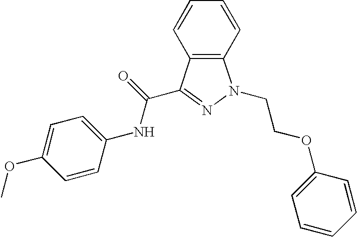

57. 1-(1-(4-((benzo[d][1,3]dioxol-5-yloxy)methyl)benzyl)-5-(trifluoromethyl)-1H-indazol-3-yl)-3-p-tolylurea;

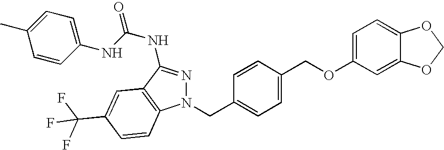

58. N-(benzo[d][1,3]dioxol-5-yl)-1-(2-phenoxyethyl)-1H-indazole-3-carboxamide;

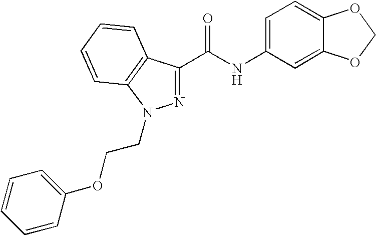

59. 1-p-tolyl-3-(1-(4-(trifluoromethyl)benzyl)-1H-indazol-3-yl)urea;

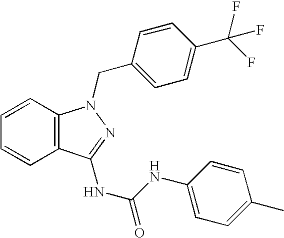

60. 1-(2-(4-fluorophenoxy)ethyl)-N-(4-isopropylphenyl)-1H-indazole-3-carboxamide;

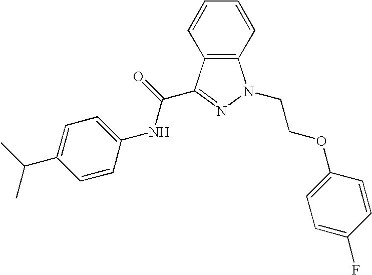

61. 1-(4-(morpholine-4-carbonyl)benzyl)-N-(4-(trifluoromethoxy)phenyl)-1H-indazole-3-carboxamide;

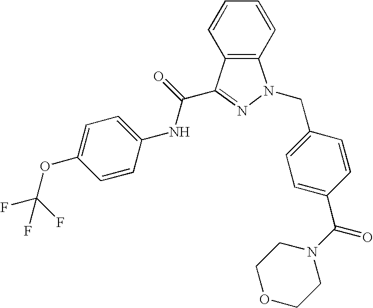

62. N-(4-isopropylphenyl)-1-(4-(methylsulfonyl)benzyl)-1H-indazole-3-carboxamide;

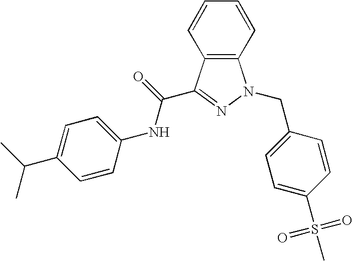

63. N-(benzo[d][1,3]dioxol-5-yl)-1-(4-(morpholine-4-carbonyl)benzyl)-1H-indazole-3-carboxamide;

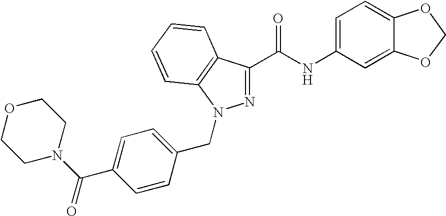

64. N-(4-carbamoylphenyl)-1-(4-(morpholinosulfonyl)benzyl)-1H-indazole-3-carboxamide;

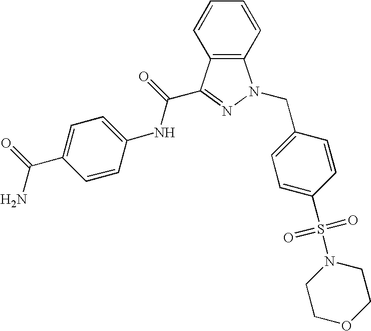

65. 1-(4-tert-butylbenzyl)-N-(4-methoxyphenyl)-1H-indazole-3-carboxamide;

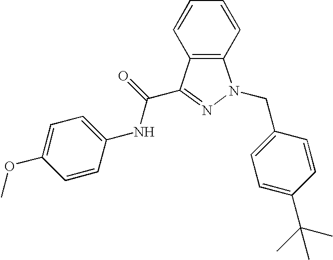

66. N-(4-isopropylphenyl)-1-(4-(N-methyl-N-(prop-2-ynyl)sulfamoyl)benzyl)-1H-indazole-3-carboxamide;

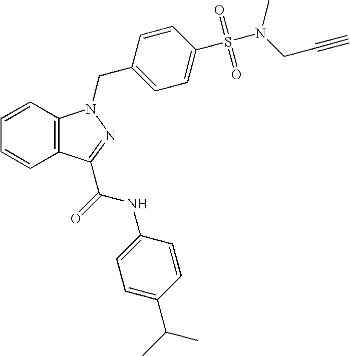

67. 1-(2-(4-fluorophenoxy)ethyl)-N-(pyridin-2-yl)-1H-indazole-3-carboxamide;

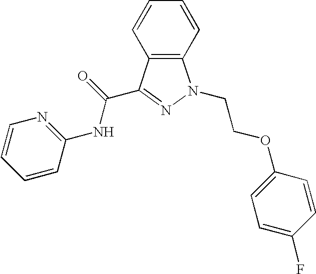

68. 1-(1-(4-((3-methoxyphenoxy)methyl)benzyl)-1H-indazol-3-yl)-3-(2-(trifluoromethoxy)phenyl)urea;

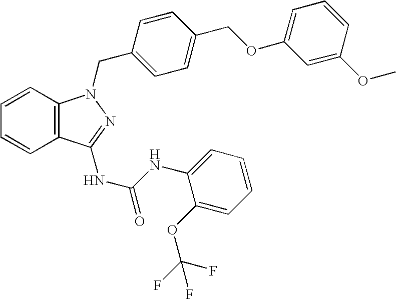

69. 1-(biphenyl-4-ylmethyl)-N-(4-(piperidin-1-yl)phenyl)-1H-indazole-3-carboxamide;

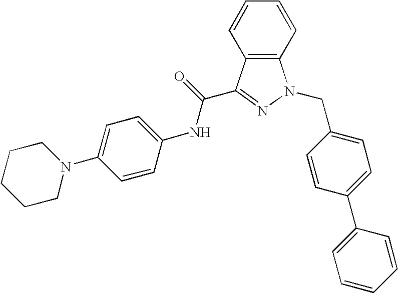

70. 1-(4-tert-butylbenzyl)-N-(4-(trifluoromethoxy)phenyl)-1H-indazole-3-carboxamide;

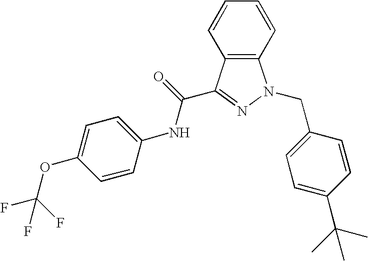

71. 1-(2-phenoxyethyl)-N-(thiazol-2-yl)-1H-indazole-3-carboxamide;

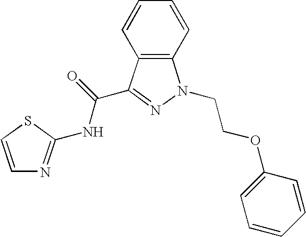

72. 1-(4-(morpholine-4-carbonyl)benzyl)-N-((tetrahydrofuran-2-yl)methyl)-1H-indazole-3-carboxamide;

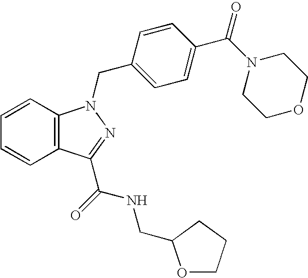

73. 1-(3,4-dimethoxyphenethyl)-3-(1-(4-(trifluoromethyl)benzyl)-1H-indazol-3-yl)urea;

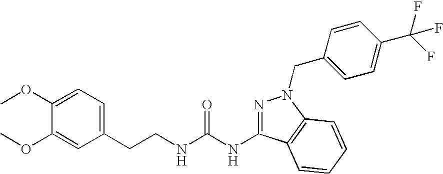

74. 1-(biphenyl-4-ylmethyl)-N-(4-methoxyphenyl)-1H-indazole-3-carboxamide;

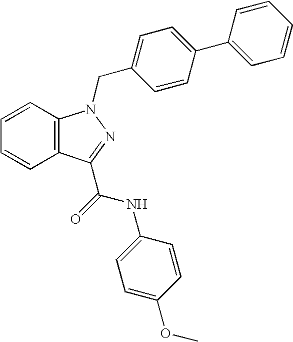

75. 1-(2-phenoxyethyl)-N-(4-(piperidin-1-yl)phenyl)-1H-indazole-3-carboxamide;

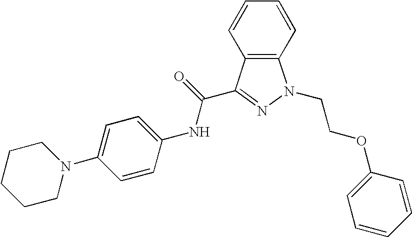

76. N-(4-carbamoylphenyl)-1-(4-(N-methyl-N-(prop-2-ynyl)sulfamoyl)benzyl)-1H-indazole-3-carboxamide;

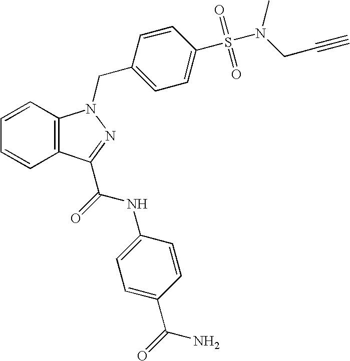

77. 1-(4-(methylsulfonyl)benzyl)-N-(pyridin-2-yl)-1H-indazole-3-carboxamide;

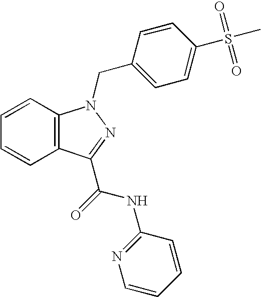

78. 1-(1-((6-chlorobenzo[d][1,3]dioxol-5-yl)methyl)-1H-indazol-3-yl)-3-(2-methoxybenzyl)urea;

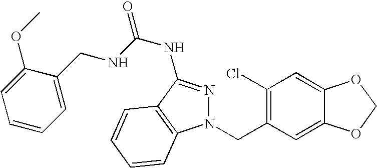

79. 1-(4-tert-butylbenzyl)-N-(4-(piperidin-1-yl)phenyl)-1H-indazole-3-carboxamide;

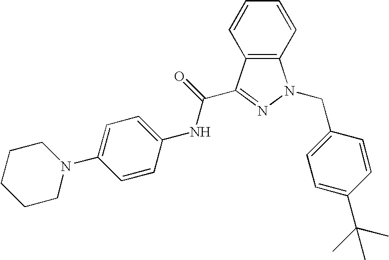

80. 1-(4-methoxyphenethyl)-3-(1-(4-((3-methoxyphenoxy)methyl)benzyl)-1H-indazol-3-yl)urea;

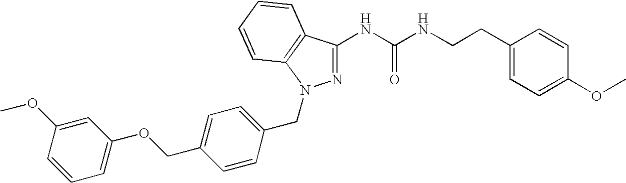

81. N-(4-tert-butylphenyl)-1-(4-(methylsulfonyl)benzyl)-1H-indazole-3-carboxamide;

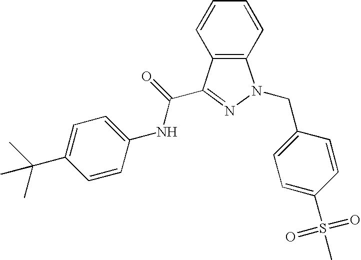

82. N-(3,4-dimethoxyphenyl)-1-(4-(methylsulfonyl)benzyl)-1H-indazole-3-carboxamide;

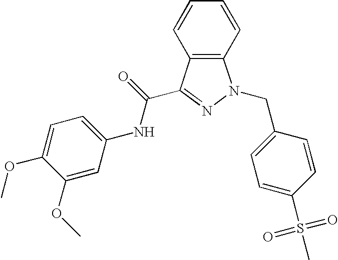

83. N-phenyl-1-(4-(methylsulfonyl)benzyl)-1H-indazole-3-carboxamide;

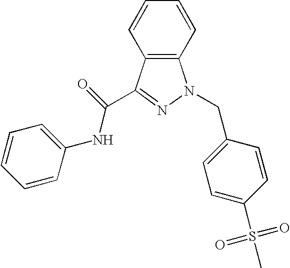

84. N-(4-ethylphenyl)-1-(4-(methylsulfonyl)benzyl)-1H-indazole-3-carboxamide;

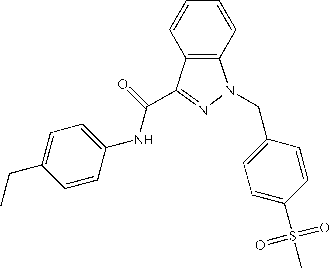

85. N-(2-isopropylphenyl)-1-(4-(methylsulfonyl)benzyl)-1H-indazole-3-carboxamide;

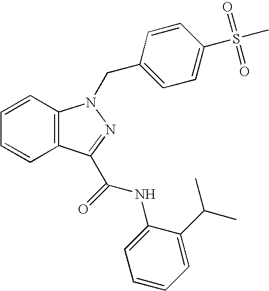

86. N-(3-methoxyphenyl)-1-(4-(methylsulfonyl)benzyl)-1H-indazole-3-carboxamide;

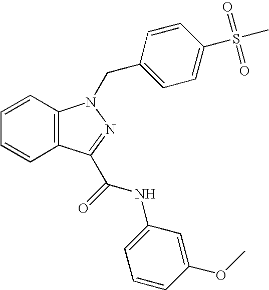

87. N-(benzo[d][1,3]dioxol-5-yl)-1-benzyl-1H-indazole-3-carboxamide;

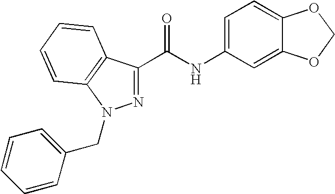

88. N-(4-hydroxyphenyl)-1-(4-(methylsulfonyl)benzyl)-1H-indazole-3-carboxamide;

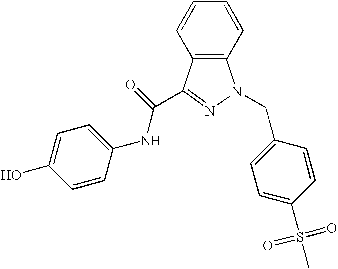

89. N-(4-chlorophenyl)-1-(4-(methylsulfonyl)benzyl)-1H-indazole-3-carboxamide;

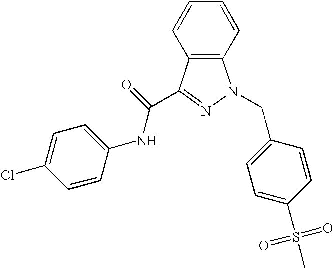

90. N-((1-(4-(methylsulfonyl)benzyl)-1H-indazol-3-yl)methyl)benzo[d][1,3]dioxol-5-amine;

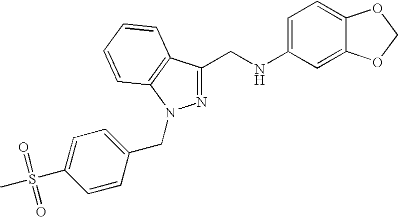

91. 1-(4-isopropylbenzyl)-N-(4-(methylsulfonyl)phenyl)-1H-indazole-3-carboxamide;

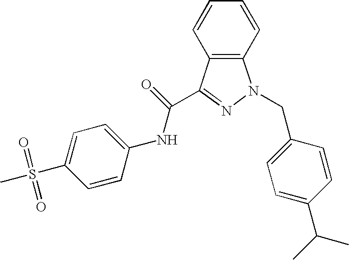

92. N-(benzo[d][1,3]dioxol-5-yl)-1-(4-(morpholine-4-carbonyl)phenethyl)-1H-indazole-3-carboxamide;

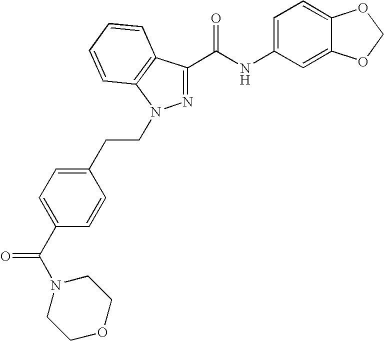

93. N-(benzo[d][1,3]dioxol-5-yl)-1-(4-(piperidine-1-carbonyl)benzyl)-1H-indazole-3-carboxamide;

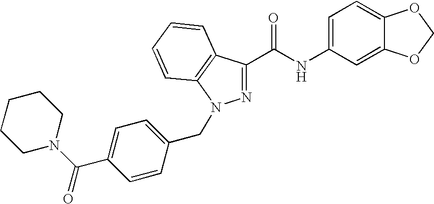

94. N-(4-fluorophenyl)-1-(4-(methylsulfonyl)benzyl)-1H-indazole-3-carboxamide;

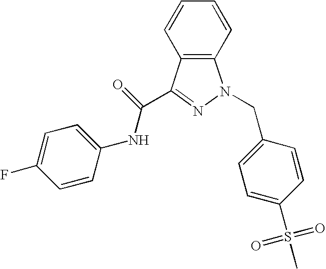

95. 6-chloro-2-(1-(4-(methylsulfonyl)benzyl)-1H-indazol-3-yl)benzo[d]oxazole;

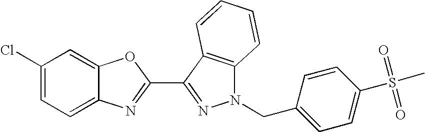

96. N-(3-isopropylphenyl)-1-(4-(methylsulfonyl)benzyl)-1H-indazole-3-carboxamide;

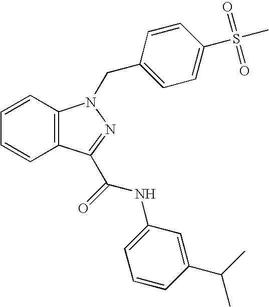

97. N-(benzo[d][1,3]dioxol-5-yl)-1-(pyridin-4-ylmethyl)-1H-indazole-3-carboxamide;

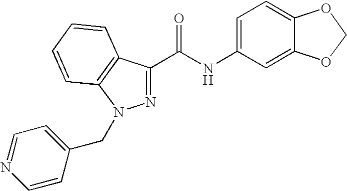

98. N-(3-isopropyl-1,2,4-thiadiazol-5-yl)-1-(4-(methylsulfonyl)benzyl)-1H-indazole-3-carboxamide;

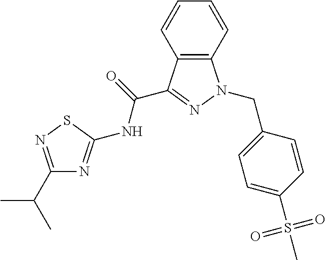

99. 1-(4-(methylsulfonyl)benzyl)-N-(quinolin-6-yl)-1H-indazole-3-carboxamide;

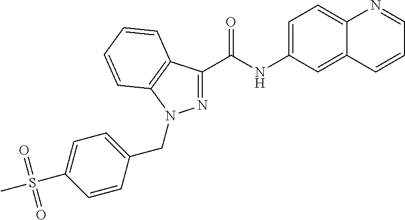

100. N-(benzo[d][1,3]dioxol-5-yl)-1-(3-(morpholine-4-carbonyl)benzyl)-1H-indazole-3-carboxamide;

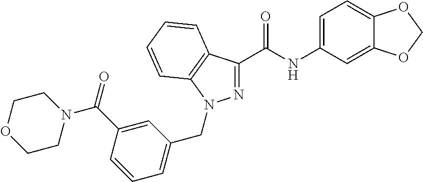

101. 1-(4-(methylsulfonyl)benzyl)-N-(4-(methylsulfonyl)phenyl)-1H-indazole-3-carboxamide;

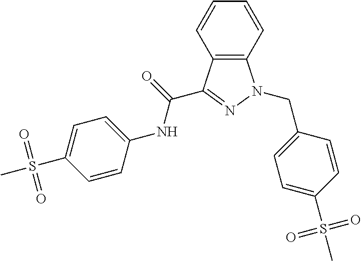

102. N-(benzo[d][1,3]dioxol-5-yl)-1-(4-(methylsulfonyl)benzyl)-1H-indazole-3-carbothioamide;

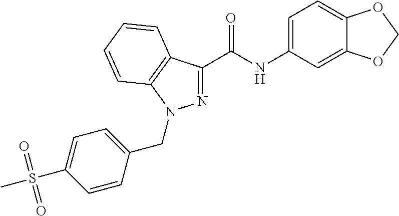

103. 1-(4-(methylsulfonyl)benzyl)-N-(quinolin-3-yl)-1H-indazole-3-carboxamide;

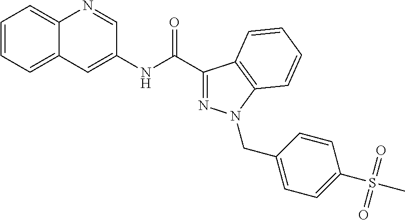

104. N-(3,4-dimethylphenyl)-1-(4-(methylsulfonyl)benzyl)-1H-indazole-3-carboxamide;

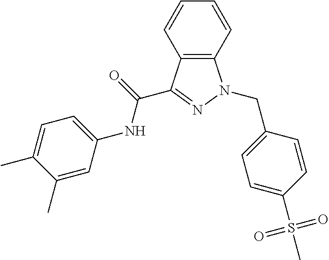

105. N-(2,3-dihydrobenzo[b][1,4]dioxin-6-yl)-1-(4-(methylsulfonyl)benzyl)-1H-indazole-3-carboxamide;

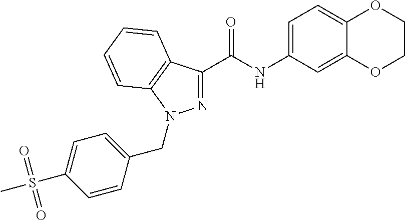

106. N-(benzo[d][1,3]dioxol-5-yl)-1-(pyridin-3-ylmethyl)-1H-indazole-3-carboxamide;

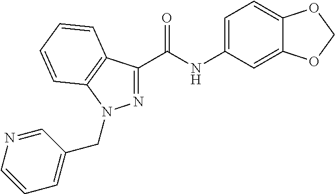

107. N-(4-acetamidophenyl)-1-(4-(methylsulfonyl)benzyl)-1H-indazole-3-carboxamide;

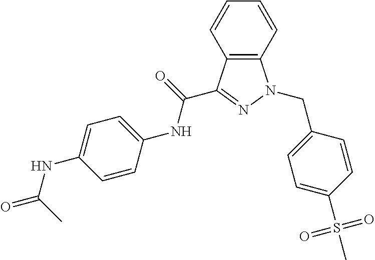

108. 1-(4-(methylsulfonyl)benzyl)-N-p-tolyl-1H-indazole-3-carboxamide;

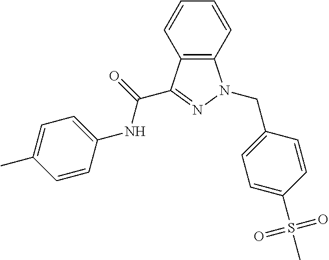

109. N-(4-acetamidophenyl)-1-(4-(trifluoromethyl)benzyl)-1H-indazole-3-carboxamide;

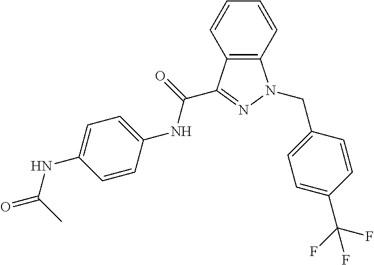

110. 1-(4-(methylsulfonyl)benzyl)-N-(quinoxalin-6-yl)-1H-indazole-3-carboxamide;

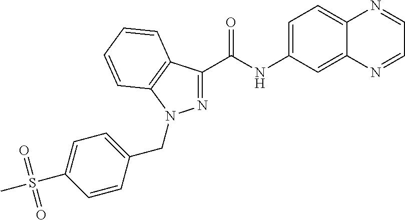

111. N-(3,4-dichlorophenyl)-1-(4-(methylsulfonyl)benzyl)-1H-indazole-3-carboxamide;

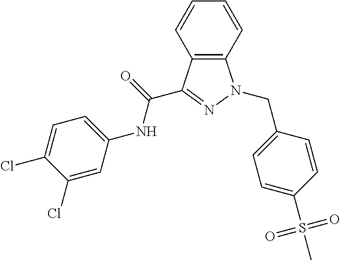

112. 1-(4-(methylsulfonyl)benzyl)-N-(4-propylphenyl)-1H-indazole-3-carboxamide;

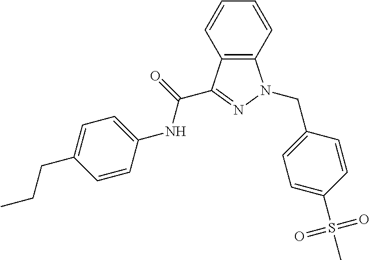

113. N-(1,3-dihydrobenzo[c]thiophen-2,2-dioxy-5-yl)-1-(4-(methylsulfonyl)benzyl)-1H-indazole-3-carboxamide;

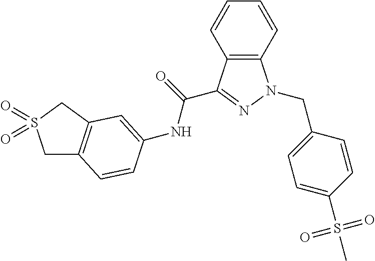

114. N-(1-(4-(methylsulfonyl)benzyl)-1H-indazol-3-yl)benzo[d][1,3]dioxole-5-carboxamide;

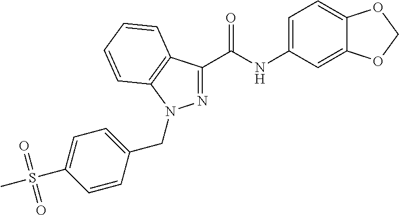

115. N-(benzo[d][1,3]dioxol-5-yl)-1-(4-(trifluoromethyl)benzyl)-1H-indazole-3-carboxamide;

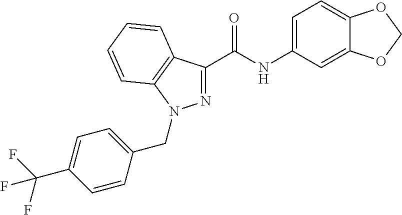

116. 1-(4-(methylsulfonyl)benzyl)-N-(naphthalen-2-yl)-1H-indazole-3-carboxamide;

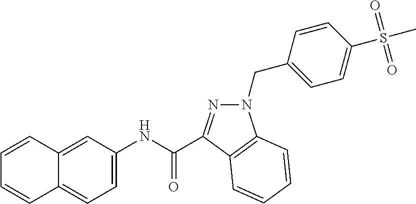

117. N-(benzo[d][1,3]dioxol-5-yl)-1-(4-(dimethylcarbamoyl)benzyl)-1H-indazole-3-carboxamide;

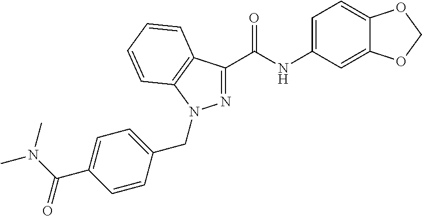

118. 1-(4-chlorophenyl)-3-(1-(4-(methylsulfonyl)benzyl)-1H-indazol-3-yl)urea;

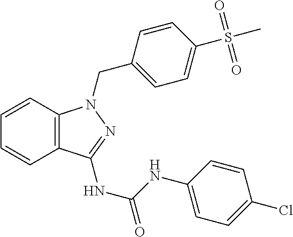

119. N-(benzo[d][1,3]dioxol-5-yl)-1-(4-isopropylbenzyl)-1H-indazole-3-carboxamide;

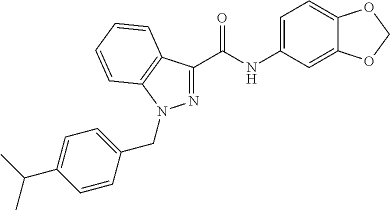

120. N-(benzofuran-5-yl)-1-(4-(methylsulfonyl)benzyl)-1H-indazole-3-carboxamide;

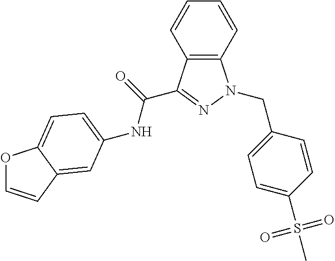

121. 1-(4-(methylsulfonyl)benzyl)-N-(5,6,7,8-tetrahydronaphthalen-2-yl)-1H-indazole-3-carboxamide;

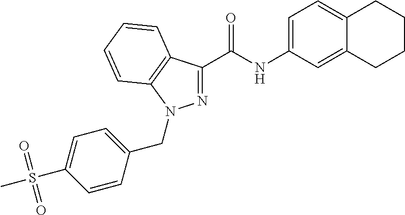

122. N-(4-cyclohexylphenyl)-1-(4-(methylsulfonyl)benzyl)-1H-indazole-3-carboxamide;

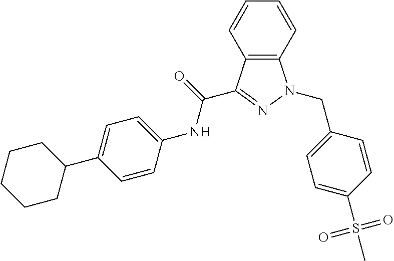

123. N-(benzo[d][1,3]dioxol-5-yl)-1-(benzo[d][1,3]dioxol-5-ylmethyl)-1H-indazole-3-carboxamide;

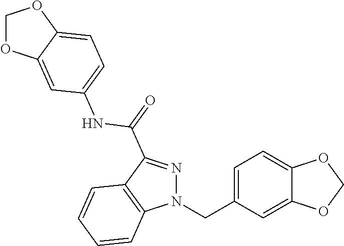

124. N-(benzo[d][1,3]dioxol-5-yl)-1-(4-tert-butylbenzoyl)-1H-indazole-3-carboxamide;

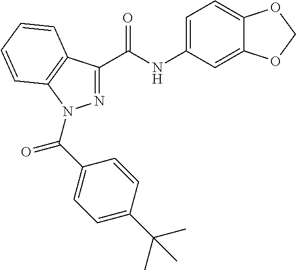

125. 1-(4-(methylsulfonyl)benzyl)-N-(5-methylthiazol-2-yl)-1H-indazole-3-carboxamide;

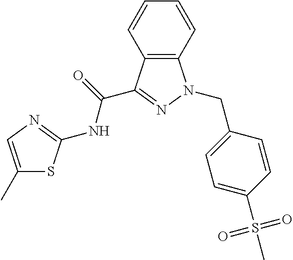

126. 1-(4-(methylsulfonyl)benzyl)-N-(4-methylthiazol-2-yl)-1H-indazole-3-carboxamide;

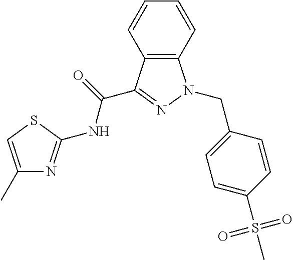

127. N-(benzo[d][1,3]dioxol-5-yl)-1-(3-(methylsulfonyl)benzyl)-1H-indazole-3-carboxamide;

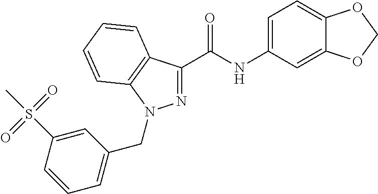

128. N-(benzo[d][1,3]dioxol-5-yl)-1-(4-(N,N-dimethylsulfamoyl)benzyl)-1H-indazole-3-carboxamide;

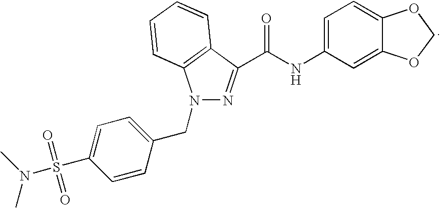

129. N-(benzo[d][1,3]dioxol-5-yl)-1-(thiophen-3-ylmethyl)-1H-indazole-3-carboxamide;

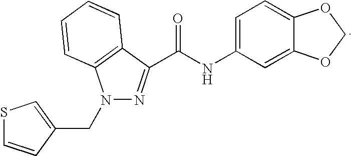

130. N-(2,3-dihydro-1H-inden-5-yl)-1-(4-(methylsulfonyl)benzyl)-1H-indazole-3-carboxamide;

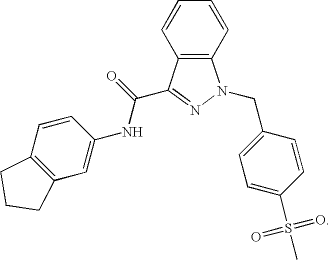

131. N-(benzo[d][1,3]dioxol-5-yl)-1-(4-methylbenzyl)-1H-indazole-3-carboxamide;

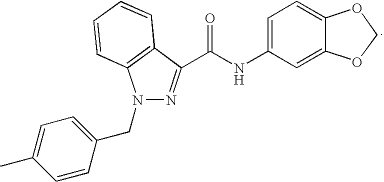

132. N-(benzo[d][1,3]dioxol-5-yl)-1-(4-ethylbenzyl)-1H-indazole-3-carboxamide;

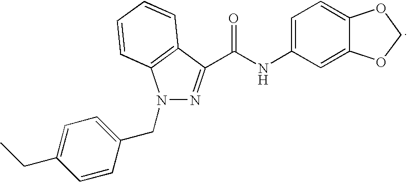

133. N-(6-methylpyridin-3-yl)-1-(4-(methylsulfonyl)benzyl)-1H-indazole-3-carboxamide;

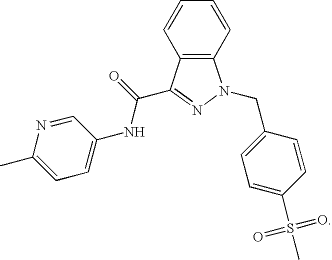

134. N-(5-methylpyridin-2-yl)-1-(4-(methylsulfonyl)benzyl)-1H-indazole-3-carboxamide;

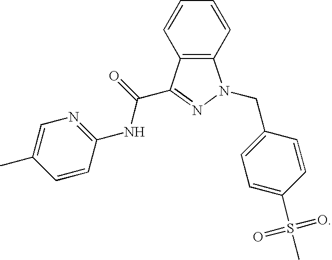

135. N-(2,2-difluorobenzo[d][1,3]dioxol-5-yl)-2-(3-(dimethylcarbamoyl)benzyl)-2H-indazole-3-carboxamide;

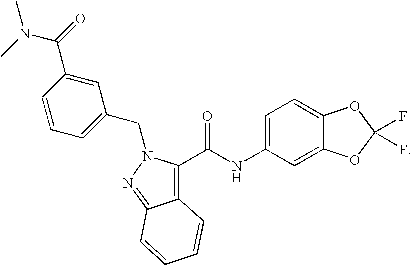

136. N-(benzo[d][1,3]dioxol-5-yl)-1-(2-(methylsulfonyl)benzyl)-1H-indazole-3-carboxamide;

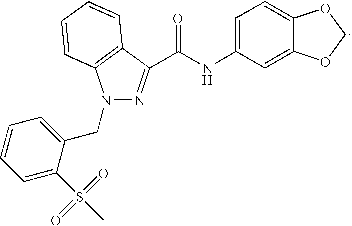

137. N-(benzo[d][1,3]dioxol-5-yl)-1-(4-(morpholinosulfonyl)benzyl)-1H-indazole-3-carboxamide;

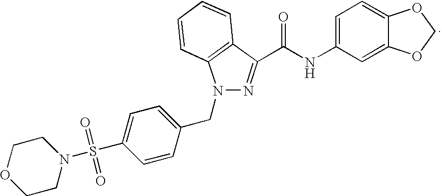

138. 1-(4-(dimethylcarbamoyl)benzyl)-N-p-tolyl-1H-indazole-3-carboxamide;

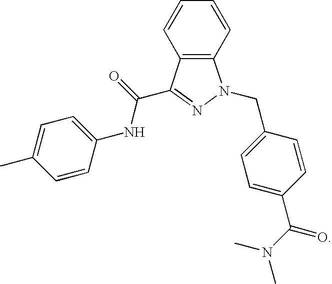

139. 1-(4-(dimethylcarbamoyl)benzyl)-N-(4-isopropylphenyl)-1H-indazole-3-carboxamide;

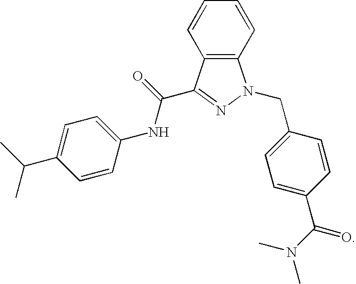

140. N-(benzo[d][1,3]dioxol-5-yl)-1-(4-(methylsulfonyl)benzoyl)-1H-indazole-3-carboxamide;

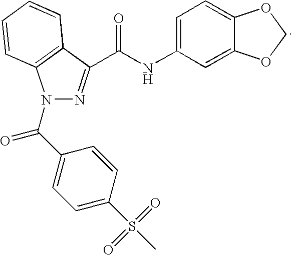

141. N-(benzo[d][1,3]dioxol-5-yl)-1-(naphthalen-2-ylmethyl)-1H-indazole-3-carboxamide;

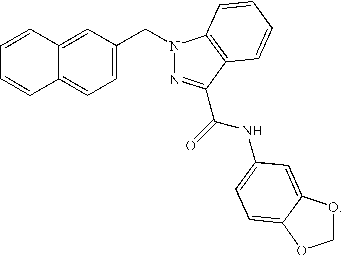

142. N-(benzo[d][1,3]dioxol-5-yl)-1-(4-(pyrrolidine-1-carbonyl)benzyl)-1H-indazole-3-carboxamide;

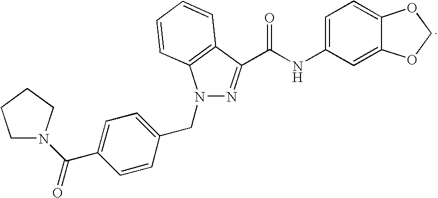

143. N-(2,3-dihydrobenzo[b][1,4]dioxin-6-yl)-1-(4-(morpholine-4-carbonyl)benzyl)-1H-indazole-3-carboxamide;

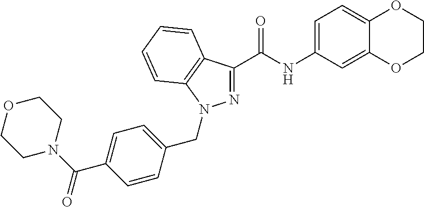

144. N-(4-methoxyphenyl)-1-(4-(morpholine-4-carbonyl)benzyl)-1H-indazole-3-carboxamide;

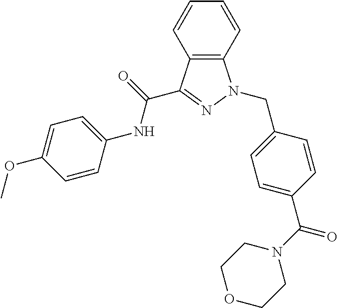

145. N-(benzo[d][1,3]dioxol-5-yl)-1-(isoxazol-3-ylmethyl)-1H-indazole-3-carboxamide;

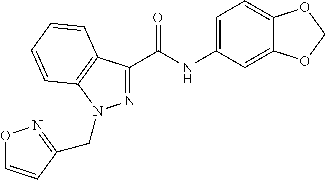

146. N-(benzo[d][1,3]dioxol-5-yl)-1-(4-(methylsulfonyl)benzyl)-1H-indole-3-carboxamide;

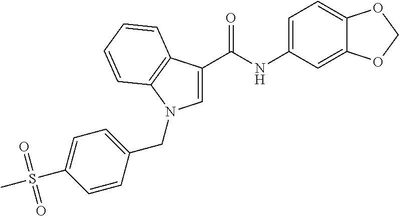

147. N-(2,4-dichlorophenyl)-1-ethyl-1H-pyrazole-3-carboxamide;

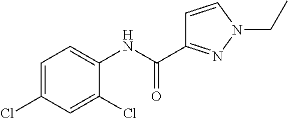

148. N-(benzo[d][1,3]dioxol-5-yl)-1-(4-(methylsulfonyl)benzyl)-1H-pyrazole-3-carboxamide;

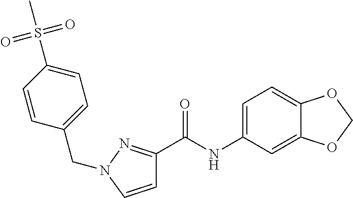

149. N-(benzo[d][1,3]dioxol-5-yl)-1-(4-(methylsulfonyl)benzyl)-4,5,6,7-tetrahydro-1H-indazole-3-carboxamide;

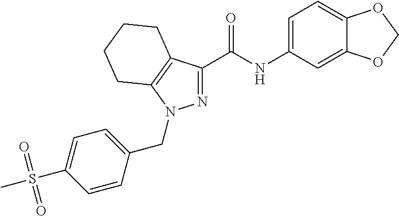

150. N-(benzo[d][1,3]dioxol-5-yl)-1-(4-(methylsulfonyl)benzyl)-1,4,5,6-tetrahydrocyclopenta[c]pyrazole-3-carboxamide;

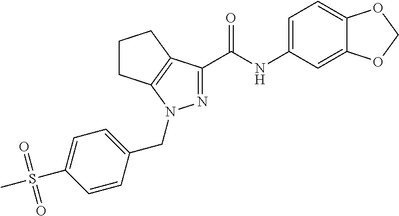

151. N-(benzo[d][1,3]dioxol-5-yl)-1-(4-(diethylcarbamoyl)benzyl)-1H-indazole-3-carboxamide;

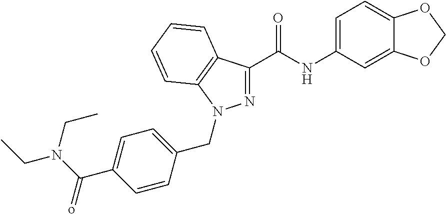

152. N-(benzo[d][1,3]dioxol-5-yl)-1-(4-(isopropylsulfonyl)benzyl)-1H-indazole-3-carboxamide;

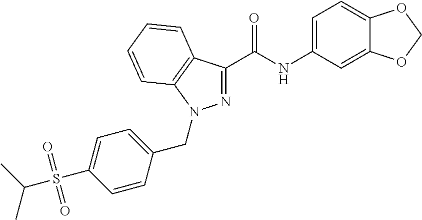

153. N-(benzo[d][1,3]dioxol-5-yl)-1-((5-ethylpyridin-2-yl)methyl)-1H-indazole-3-carboxamide;

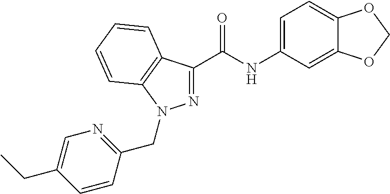

154. N-(benzo[d][1,3]dioxol-5-yl)-1-((5-isopropyl-1,2,4-oxadiazol-3-yl)methyl)-1H-indazole-3-carboxamide;

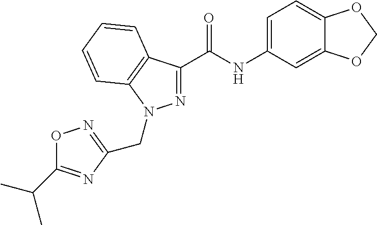

155. N-(benzo[d][1,3]dioxol-5-yl)-1-(5-ethyl-1,3,4-oxadiazol-2-yl)methyl)-1H-indazole-3-carboxamide;

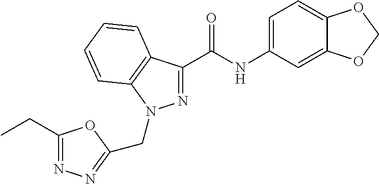

156. N-(benzo[d][1,3]dioxol-5-yl)-1-(4-(ethylsulphonyl)benzyl)-1H-indazole-3-carboxamide;

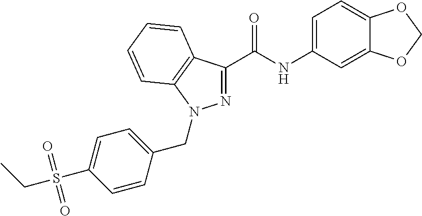

157. 1-(4-(methylsulphonyl)benzyl)-1H-indazole-3-carboxylic acid;

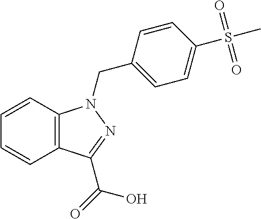

158. N-(2,3-dihydrobenzo[b][1,4]dioxin-6-yl)-1-(4-ethylbenzyl)-1H-indazole-3-carboxamide;

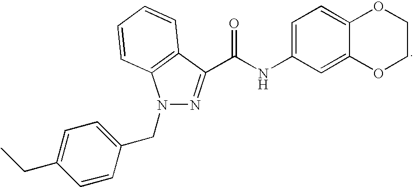

159. 1-(4-ethylbenzyl)-N-(2,2,3,3-tetrafluoro-2,3-dihydrobenzo[b][1,4]dioxin-6-yl)-1H-indazole-3-carboxamide;

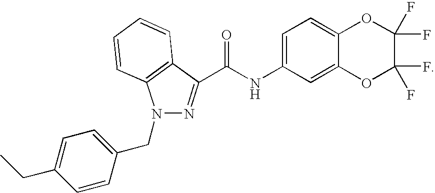

160. N-(2,3-dihydrobenzo[b][1,4]dioxin-6-yl)-5-fluoro-1-(4-(morpholine-4-carbonyl)benzyl)-1H-indazole-3-carboxamide;

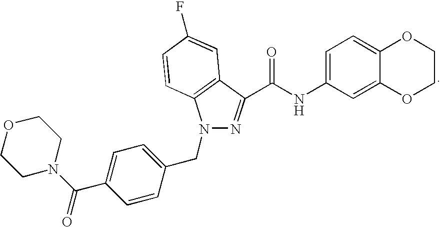

161. 1-(4-(morpholine-4-carbonyl)benzyl)-N-(2,2,3,3-tetrafluoro-2,3-dihydrobenzo[b][1,4]dioxin-6-yl)-1H-indazole-3-carboxamide;

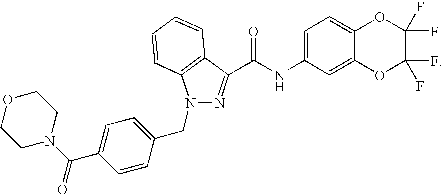

162. N-(2,3-dihydrobenzo[b][1,4]dioxin-6-yl)-6-fluoro-1-(4-(morpholine-4-carbonyl)benzyl)-1H-indazole-3-carboxamide;

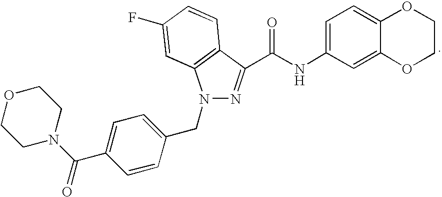

163. N-(2,3-dihydrobenzo[b][1,4]dioxin-6-yl)-1-(4-(morpholine-4-carbonyl)benzyl)-6-(trifluoromethyl)-1H-indazole-3-carboxamide;

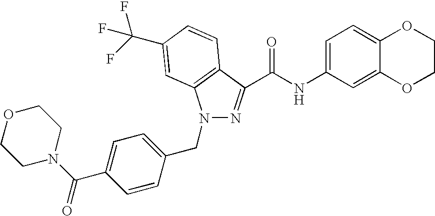

or a tautomer, enantiomer, pharmaceutically acceptable salt, hydrate, solvate, complex, or prodrug thereof.

In another embodiment, specific examples of compounds of formula (I) include, but are not limited to, the following:
N-(4-tert-butylphenyl)-1-(4-(methylsulfonyl)benzyl)-1H-indazole-3-carboxamide;
N-(3,4-dimethoxyphenyl)-1-(4-(methylsulfonyl)benzyl)-1H-indazole-3-carboxamide;
N-phenyl-1-(4-(methylsulfonyl)benzyl)-1H-indazole-3-carboxamide;
N-(4-ethylphenyl)-1-(4-(methylsulfonyl)benzyl)-1H-indazole-3-carboxamide;
N-(2-isopropylphenyl)-1-(4-(methylsulfonyl)benzyl)-1H-indazole-3-carboxamide;
N-(3-methoxyphenyl)-1-(4-(methylsulfonyl)benzyl)-1H-indazole-3-carboxamide;
N-(benzo[d][1,3]dioxol-5-yl)-1-benzyl-1H-indazole-3-carboxamide;
N-(4-hydroxyphenyl)-1-(4-(methylsulfonyl)benzyl)-1H-indazole-3-carboxamide;
N-(4-chlorophenyl)-1-(4-(methylsulfonyl)benzyl)-1H-indazole-3-carboxamide;
N-((1-(4-(methylsulfonyl)benzyl)-1H-indazol-3-yl)methyl)benzo[d][1,3]dioxol-5-amine;
1-(4-isopropylbenzyl)-N-(4-(methylsulfonyl)phenyl)-1H-indazole-3-carboxamide;
N-(benzo[d][1,3]dioxol-5-yl)-1-(4-(morpholine-4-carbonyl)phenethyl)-1H-indazole-3-carboxamide;
N-(benzo[d][1,3]dioxol-5-yl)-1-(4-(piperidine-1-carbonyl)benzyl)-1H-indazole-3-carboxamide;
N-(4-fluorophenyl)-1-(4-(methylsulfonyl)benzyl)-1H-indazole-3-carboxamide;
6-chloro-2-(1-(4-(methylsulfonyl)benzyl)-1H-indazol-3-yl)benzo[d]oxazole;
N-(3-isopropylphenyl)-1-(4-(methylsulfonyl)benzyl)-1H-indazole-3-carboxamide;
N-(benzo[d][1,3]dioxol-5-yl)-1-(pyridin-4-ylmethyl)-1H-indazole-3-carboxamide;
N-(3-isopropyl-1,2,4-thiadiazol-5-yl)-1-(4-(methylsulfonyl)benzyl)-1H-indazole-3-carboxamide;
1-(4-(methylsulfonyl)benzyl)-N-(quinolin-6-yl)-1H-indazole-3-carboxamide;
N-(benzo[d][1,3]dioxol-5-yl)-1-(3-(morpholine-4-carbonyl)benzyl)-1H-indazole-3-carboxamide;
1-(4-(methylsulfonyl)benzyl)-N-(4-(methylsulfonyl)phenyl)-1H-indazole-3-carboxamide;
N-(benzo[d][1,3]dioxol-5-yl)-1-(4-(methylsulfonyl)benzyl)-1H-indazole-3-carbothioamide;
1-(4-(methylsulfonyl)benzyl)-N-(quinolin-3-yl)-1H-indazole-3-carboxamide;
N-(3,4-dimethylphenyl)-1-(4-(methylsulfonyl)benzyl)-1H-indazole-3-carboxamide;
N-(2,3-dihydrobenzo[b][1,4]dioxin-6-yl)-1-(4-(methylsulfonyl)benzyl)-1H-indazole-3-carboxamide;
N-(benzo[d][1,3]dioxol-5-yl)-1-(pyridin-3-ylmethyl)-1H-indazole-3-carboxamide;
N-(4-acetamidophenyl)-1-(4-(methylsulfonyl)benzyl)-1H-indazole-3-carboxamide;
1-(4-(methylsulfonyl)benzyl)-N-p-tolyl-1H-indazole-3-carboxamide;
N-(4-acetamidophenyl)-1-(4-(trifluoromethyl)benzyl)-1H-indazole-3-carboxamide;
1-(4-(methylsulfonyl)benzyl)-N-(quinoxalin-6-yl)-1H-indazole-3-carboxamide;
N-(3,4-dichlorophenyl)-1-(4-(methylsulfonyl)benzyl)-1H-indazole-3-carboxamide;
1-(4-(methylsulfonyl)benzyl)-N-(4-propylphenyl)-1H-indazole-3-carboxamide;
N-(1,3-dihydrobenzo[c]thiophen-2,2-dioxy-5-yl)-1-(4-(methylsulfonyl)benzyl)-1H-indazole-3-carboxamide;
N-(1-(4-(methylsulfonyl)benzyl)-1H-indazol-3-yl)benzo[d][1,3]dioxole-5-carboxamide;
N-(benzo[d][1,3]dioxol-5-yl)-1-(4-(trifluoromethyl)benzyl)-1H-indazole-3-carboxamide;
1-(4-(methylsulfonyl)benzyl)-N-(naphthalen-2-yl)-1H-indazole-3-carboxamide;
N-(benzo[d][1,3]dioxol-5-yl)-1-(4-(dimethylcarbamoyl)benzyl)-1H-indazole-3-carboxamide;
1-(4-chlorophenyl)-3-(1-(4-(methylsulfonyl)benzyl)-1H-indazol-3-yl)urea;
N-(benzo[d][1,3]dioxol-5-yl)-1-(4-isopropylbenzyl)-1H-indazole-3-carboxamide;
N-(benzofuran-5-yl)-1-(4-(methylsulfonyl)benzyl)-1H-indazole-3-carboxamide;
1-(4-(methylsulfonyl)benzyl)-N-(5,6,7,8-tetrahydronaphthalen-2-yl)-1H-indazole-3-carboxamide;
N-(4-cyclohexylphenyl)-1-(4-(methylsulfonyl)benzyl)-1H-indazole-3-carboxamide;
N-(benzo[d][1,3]dioxol-5-yl)-1-(benzo[d][1,3]dioxol-5-ylmethyl)-1H-indazole-3-carboxamide;
N-(benzo[d][1,3]dioxol-5-yl)-1-(4-tert-butylbenzoyl)-1H-indazole-3-carboxamide;
1-(4-(methylsulfonyl)benzyl)-N-(5-methylthiazol-2-yl)-1H-indazole-3-carboxamide;
1-(4-(methylsulfonyl)benzyl)-N-(4-methylthiazol-2-yl)-1H-indazole-3-carboxamide;
N-(benzo[d][1,3]dioxol-5-yl)-1-(3-(methylsulfonyl)benzyl)-1H-indazole-3-carboxamide;
N-(benzo[d][1,3]dioxol-5-yl)-1-(4-(N,N-dimethylsulfamoyl)benzyl)-1H-indazole-3-carboxamide;
N-(benzo[d][1,3]dioxol-5-yl)-1-(thiophen-3-ylmethyl)-1H-indazole-3-carboxamide;
N-(2,3-dihydro-1H-inden-5-yl)-1-(4-(methylsulfonyl)benzyl)-1H-indazole-3-carboxamide;
N-(benzo[d][1,3]dioxol-5-yl)-1-(4-methylbenzyl)-1H-indazole-3-carboxamide;
N-(benzo[d][1,3]dioxol-5-yl)-1-(4-ethylbenzyl)-1H-indazole-3-carboxamide;
N-(6-methylpyridin-3-yl)-1-(4-(methylsulfonyl)benzyl)-1H-indazole-3-carboxamide;
N-(5-methylpyridin-2-yl)-1-(4-(methylsulfonyl)benzyl)-1H-indazole-3-carboxamide;
N-(2,2-difluorobenzo[d][1,3]dioxol-5-yl)-2-(3-(dimethylcarbamoyl)benzyl)-2H-indazole-3-carboxamide;
N-(benzo[d][1,3]dioxol-5-yl)-1-(2-(methylsulfonyl)benzyl)-1H-indazole-3-carboxamide;
N-(benzo[d][1,3]dioxol-5-yl)-1-(4-(morpholinosulfonyl)benzyl)-1H-indazole-3-carboxamide;
1-(4-(dimethylcarbamoyl)benzyl)-N-p-tolyl-1H-indazole-3-carboxamide;
1-(4-(dimethylcarbamoyl)benzyl)-N-(4-isopropylphenyl)-1H-indazole-3-carboxamide;
N-(benzo[d][1,3]dioxol-5-yl)-1-(4-(methylsulfonyl)benzoyl)-1H-indazole-3-carboxamide;
N-(benzo[d][1,3]dioxol-5-yl)-1-(naphthalen-2-ylmethyl)-1H-indazole-3-carboxamide;
N-(benzo[d][1,3]dioxol-5-yl)-1-(4-(pyrrolidine-1-carbonyl)benzyl)-1H-indazole-3-carboxamide;
N-(2,3-dihydrobenzo[b][1,4]dioxin-6-yl)-1-(4-(morpholine-4-carbonyl)benzyl)-1H-indazole-3-carboxamide;
N-(4-methoxyphenyl)-1-(4-(morpholine-4-carbonyl)benzyl)-1H-indazole-3-carboxamide;
N-(benzo[d][1,3]dioxol-5-yl)-1-(isoxazol-3-ylmethyl)-1H-indazole-3-carboxamide;
N-(benzo[d][1,3]dioxol-5-yl)-1-(4-(methylsulfonyl)benzyl)-1H-indole-3-carboxamide;
N-(2,4-dichlorophenyl)-1-ethyl-1H-pyrazole-3-carboxamide;
N-(benzo[d][1,3]dioxol-5-yl)-1-(4-(methylsulfonyl)benzyl)-1H-pyrazole-3-carboxamide;
N-(benzo[d][1,3]dioxol-5-yl)-1-(4-(methylsulfonyl)benzyl)-4,5,6,7-tetrahydro-1H-indazole-3-carboxamide;
N-(benzo[d][1,3]dioxol-5-yl)-1-(4-(methylsulfonyl)benzyl)-1,4,5,6-tetrahydrocyclopenta[c]pyrazole-3-carboxamide;
N-(benzo[d][1,3]dioxol-5-yl)-1-(4-(diethylcarbamoyl)benzyl)-1H-indazole-3-carboxamide;
N-(benzo[d][1,3]dioxol-5-yl)-1-(4-(isopropylsulfonyl)benzyl)-1H-indazole-3-carboxamide;
N-(benzo[d][1,3]dioxol-5-yl)-1-((5-ethylpyridin-2-yl)methyl)-1H-indazole-3-carboxamide;
N-(benzo[d][1,3]dioxol-5-yl)-1-((5-isopropyl-1,2,4-oxadiazol-3-yl)methyl)-1H-indazole-3-carboxamide;
N-(benzo[d][1,3]dioxol-5-yl)-1-((5-ethyl-1,3,4-oxadiazol-2-yl)methyl)-1H-indazole-3-carboxamide;
N-(benzo[d][1,3]dioxol-5-yl)-1-(4-(ethylsulphonyl)benzyl)-1H-indazole-3-carboxamide;
1-(4-(methylsulphonyl)benzyl)-1H-indazole-3-carboxylic acid;
N-(2,3-dihydrobenzo[b][1,4]dioxin-6-yl)-1-(4-ethylbenzyl)-1H-indazole-3-carboxamide;
1-(4-ethylbenzyl)-N-(2,2,3,3-tetrafluoro-2,3-dihydrobenzo[b][1,4]dioxin-6-yl)-1H-indazole-3-carboxamide;
N-(2,3-dihydrobenzo[b][1,4]dioxin-6-yl)-5-fluoro-1-(4-(morpholine-4-carbonyl)benzyl)-1H-indazole-3-carboxamide;
1-(4-(morpholine-4-carbonyl)benzyl)-N-(2,2,3,3-tetrafluoro-2,3-dihydrobenzo[b][1,4]dioxin-6-yl)-1H-indazole-3-carboxamide;
N-(2,3-dihydrobenzo[b][1,4]dioxin-6-yl)-6-fluoro-1-(4-(morpholine-4-carbonyl)benzyl)-1H-indazole-3-carboxamide; or
N-(2,3-dihydrobenzo[b][1,4]dioxin-6-yl)-1-(4-(morpholine-4-carbonyl)benzyl)-6-(trifluoromethyl)-1H-indazole-3-carboxamide;
   or a tautomer, enantiomer, pharmaceutically acceptable salt, hydrate, solvate, complex, or prodrug thereof.

In another embodiment, specific examples of compounds of formula (I) include the following:
1-(4-(Morpholine-4-carbonyl)benzyl)-N-(4-(trifluoromethoxy)phenyl)-1H-indazole-3-carboxamide (Compound 61);
N-(3,4-Dimethoxyphenyl)-1-(4-(methylsulfonyl)benzyl)-1H-indazole-3-carboxamide (Compound 82);
N-(Benzo[d][1,3]dioxol-5-yl)-1-(4-(methylsulfonyl)benzyl)-1,4,5,6-tetrahydrocyclopenta[c]pyrazole-3-carboxamide (Compound 150);
N-(Benzo[d][1,3]dioxol-5-yl)-1-(4-(methylsulfonyl)benzyl)-1H-indole-3-carboxamide (Compound 146); and
N-(4-Methoxyphenyl)-1-(4-(morpholine-4-carbonyl)benzyl)-1H-indazole-3-carboxamide (Compound 144).

Also provided herein is the use of a compound of formula (I) in the preparation of an agent for the treatment or prophylaxis of Duchenne muscular dystrophy, Becker muscular dystrophy, or cachexia.

Some of the examples of compounds of formula (I) are commercially available. For example, Compounds 1-80 were obtained from Biofocus Discovery Limited, Chesterford Park, Saffron Walden, Essex, and CB10 1 XL. Other examples of compounds of formula (I), for example, Compounds 81 to 163, can be synthesised from commercially available starting materials using the following methods.

### C. Synthesis of the Compounds

In one embodiment, provided herein is a method of making a compound of formula (I).

Compounds of formula (I) in which L¹ is CONH may be prepared by reacting a compound of formula (II):

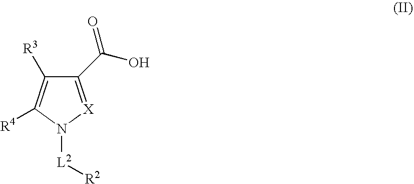

wherein R², R³, R⁴, L² and X are as defined herein elsewhere;
by reaction with a compound of formula (III):

R¹—NH₂ (III)

wherein R¹ is as defined herein elsewhere.

The reaction may be carried out in a polar organic solvent such as N,N-dimethylformamide and under basic conditions. This reaction is exemplified in procedures D, J, N and P in the examples.

Compounds of formula (III) are known and are commercially available or may be prepared by methods familiar to those of skill in the art.

Compounds of formula (II) may be prepared by the hydrolysis of an ester of formula (IV):

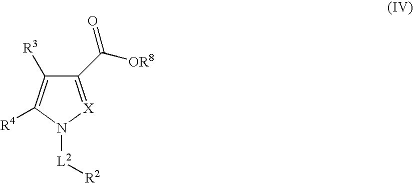

wherein R², R³, R⁴, L² and X are as defined herein elsewhere, and R⁸ is C₁-C₆ alkyl, including but not limited to, methyl or ethyl, or benzyl.

The hydrolysis may be alkaline hydrolysis achieved by the reaction of the ester with a base such as aqueous sodium hydroxide in an organic solvent, and the process is exemplified in Procedure C in the examples.

Esters of formula (IV) may be prepared from a compound of formula (V):

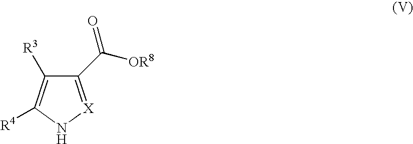

wherein R³, R⁴ and X are as defined herein elsewhere, and R⁸ is as defined for formula (IV); by reaction with a compound of formula (VI):

Cl-L²-R² (VI)

wherein L² and R² are as defined herein elsewhere.

The reaction may be conducted under basic conditions and at elevated temperature, such as, for example, 100 to 150° C. The reaction may be carried out in a polar organic solvent, such as N,N-dimethylformamide, and heating may be achieved by microwave radiation. The process is exemplified in Procedure B of the Examples.

An ester of formula (V) may also be prepared by reacting an acid of formula (VII):

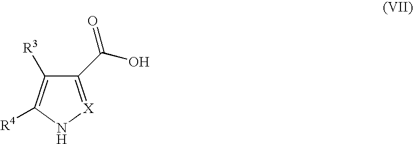

wherein R³, R⁴ and X are as defined herein elsewhere; by reaction with an appropriate alcohol of formula (VIII):

R⁸—OH (VIII)

wherein R⁸ is defined herein elsewhere. The process is exemplified in Procedure A of the Examples.

Compounds of formula (VII) and the alcohols of formula (VIII) are known to those of skill in the art and are commercially available or can be prepared by known methods.

Alternatively, compounds of formula (I) can be prepared by reacting a compound of formula (II) as defined herein elsewhere with a carbamate of formula (IX):

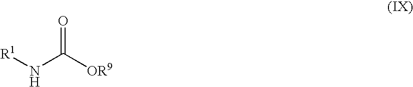

wherein R¹ is as defined herein elsewhere, and R⁹ is a group such as ^{t}butyl or benzyl. In this reaction, the carbamate (IX) is first treated with an acid, for example trifluoroacetic acid, in a polar organic solvent such as dichloromethane, followed by the addition of the compound of formula (II). This process is exemplified in procedure F in the Examples.

A carbamate of formula (IX) may be prepared by reacting a carboxylic acid of formula (X):

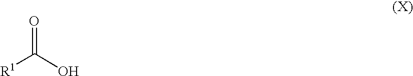

wherein R¹ is as defined herein elsewhere, with diphenylphosphoryl azide, followed by triethylamine and an alcohol of formula (XIV):

R⁹—OH (XIV)

wherein R⁹ is as defined for formula (IX). This process is exemplified in Procedure E in the Examples.

Carboxylic acids of formula (X) are commercially available or can be prepared by processes known to those of skill in the art.

Compounds of formula (I) wherein L¹ is C(O)NH and L² is C(O) may be prepared by reacting a compound of formula (XI):

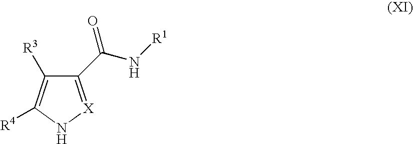

wherein X, R¹, R³ and R⁴ are as defined herein elsewhere, with a compound of formula (XII):

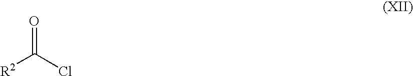

wherein R² is as defined herein elsewhere.

The reaction may be conducted in a polar organic solvent, such as N,N-dimethylformamide, and in the presence of a base, such as sodium hydride. For example, the reaction mixture may be cooled initially, for example, to 0° C., and subsequently allowed to warm to room temperature. This reaction is exemplified in the synthesis of Compound 124.

A compound of formula (XI) may be prepared by reacting a compound of formula (VII) as defined herein elsewhere with a compound of formula (III) as defined herein elsewhere using procedure D as described in the Examples below.

Compounds of formula (I) in which L¹ is NHC(O) may be prepared by reacting a compound of formula (XIII):

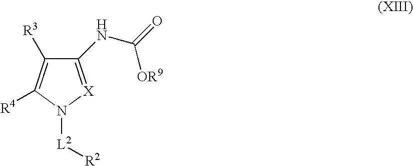

wherein R², R³, R⁴, L² and X are as defined herein elsewhere, and R⁹ is as defined for formula (IX); with a carbamate of formula (IX) as defined above. The process may be carried out according to procedure F as described in the Examples, and is illustrated in the preparation of Compound 114.

A compound of formula (XIII) may be prepared by reacting a compound of formula (II) with diphenylphosphoryl azide followed by triethylamine and an alcohol of formula (XIV):

R⁹—OH (XIV)

wherein R⁹ is as defined for formula (IX). This process is exemplified in Procedure E in the Examples.

A compound of formula (I) in which L¹ is a bond may be prepared by a variety of cyclization reactions. For example, when R¹ is a 2-benzo[d]oxazole derivative, a compound of formula (I) may be prepared by reacting a carboxylic acid derivative of formula (II) as defined herein elsewhere with optionally substituted 2-aminophenol. The starting materials react via an elimination and cyclisation reaction to give the required compound of formula (I). The reaction maybe carried out under acidic conditions and at raised temperature, for example, 100 to 150° C. An example of such a reaction is described in the Examples under Procedure H.

Compounds of formula (I) in which L¹ is NHC(O)NH may be prepared from compounds of formula (XIII) as defined herein elsewhere, by reaction with a compound of formula (XV):

R¹—N═C═O (XV)

wherein R¹ is as defined herein elsewhere. The reaction may be carried out at room temperature and under basic conditions. An example of the process is described in procedure L of the Examples.

Compounds of formula (I) can be converted to other compounds of formula (I). For example, a compound of formula (I) in which L¹ is CH₂NH may be prepared from a compound of formula (I) in which L¹ is C(O)NH, by reduction with borane, for example in the form of borane tetrahydrofuran complex. This reaction is exemplified in Procedure G in the Examples.

Compounds of formula (I) in which L¹ is C(S)NH may be prepared from a compound of formula (I) in which L¹ is C(O)NH, by reaction with Lawesson's reagent. The reaction may be carried out in an organic solvent, for example a mixture of toluene and 1,4-dioxane. This reaction is exemplified in Procedure K in the Examples.

In the syntheses described herein, suitable protecting groups may be used, as known to one skilled in the art. One skilled in the art would be able to choose appropriate protecting groups and conditions to introduce and remove such protecting groups. Information concerning protecting groups is available in “Protecting Groups in Organic Synthesis”, Theodora W. Greene and Peter G. M. Wuts, published by John Wiley & Sons Inc.

### D. Pharmaceutical Compositions

In one embodiment, the compounds of formula (I) for use in the treatment of DMD is administered in the form of a pharmaceutical composition. Provided herein is a pharmaceutical composition comprising a compound of formula (I), or its tautomer, enantiomer, pharmaceutically acceptable salt, hydrate, solvate, complex, or prodrug thereof; and one or more pharmaceutically acceptable excipients.

In one embodiment, the pharmaceutical composition comprises less than 80% w/w, less than 50% w/w, less than 20% w/w, or between 0.1 to 20% w/w, of a compound of formula (I), or a pharmaceutically acceptable salt thereof, in admixture with a pharmaceutically acceptable diluent or carrier.

In one embodiment, provided herein is a process for the production of such a pharmaceutical composition, which comprises mixing the ingredients.

In one embodiment, examples of pharmaceutical formulation, compositions, and suitable diluents or carriers, include, but are not limited to, the following:

for intravenous injection or infusion—purified water or saline solution;
for inhalation compositions—coarse lactose;
for tablets, capsules and dragees—microcrystalline cellulose, calcium phosphate, diatomaceous earth, a sugar such as lactose, dextrose or mannitol, talc, stearic acid, starch, sodium bicarbonate and/or gelatin;
for suppositories—natural or hardened oils or waxes.

In one embodiment, the compound is used in an aqueous solution, for example in intravenous infusion, the pharmaceutical composition comprising the compound may further comprise one or more excipients. In certain embodiment, the excipients include, but are not limited to, chelating or sequestering agents, antioxidants, tonicity adjusting agents, pH-modifying agents, or buffering agents.

In one embodiment, solutions containing a compound of formula (I) may, if desired, be evaporated, for example, by freeze drying or spray drying, to give a solid composition, which may be reconstituted prior to use.

In one embodiment, the compound of formula (I) is not used as a solution. In one embodiment, the compound of formula (I) is in a form having a mass median diameter of from 0.01 to 10 μm. In certain embodiment, the pharmaceutical composition comprising a compound of formula (I) may further contain preserving, stabilizing, and wetting agents, solubilisers, for example, a water-soluble cellulose polymer such as hydroxypropyl methylcellulose, or a water-soluble glycol such as propylene glycol, sweetening and colouring agents and flavourings. In one embodiment, the compositions may be formulated in sustained release form.

In one embodiment, the pharmaceutical composion comprises a compound of formula (I) in about 0.01% to about 99.9% w/w, relative to the entire preparation. In certain embodiment, the pharmaceutical composition comprises a compound of formula (I) in about 0.1% to about 50% w/w, relative to the entire preparation.

Provided herein is a pharmaceutical composition comprising a compound of formula (I), and a pharmaceutically acceptable excipient, adjuvant, carrier, buffer, or stabiliser.

In one embodiment, the pharmaceutically acceptable excipient, adjuvant, carrier, buffer, or stabiliser is non-toxic and does not interfere with the efficacy of the active ingredient. The precise nature of the carrier or other material will depend on the route of administration, which may be oral or by injection, such as cutaneous, subcutaneous, or intravenous injection.

In one embodiment, the pharmaceutical compositions are provided in a dosage form for oral administration, which comprise a compound provided herein, and one or more pharmaceutically acceptable excipients or carriers. The pharmaceutical compositions provided herein that are formulated for oral administration may be in tablet, capsule, powder, or liquid form. A tablet may comprise a solid carrier or an adjuvant. Liquid pharmaceutical compositions generally comprise a liquid carrier such as water, petroleum, animal or vegetable oils, or mineral oil or synthetic oil. Physiological saline solution, dextrose or other saccharide solution, or glycols such as ethylene glycol, propylene glycol, or polyethylene glycol may be included. A capsule may comprise a solid carrier such as gelatin.

In another embodiment, the pharmaceutical compositions are provided in a dosage form for parenteral administration, and one or more pharmaceutically acceptable excipients or carriers. Where pharmaceutical compositions may be formulated for intravenous, cutaneous or subcutaneous injection, the active ingredient will be in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. Preservatives, stabilisers, buffers, antioxidants, and/or other additives may be included as required.

In yet another embodiment, the pharmaceutical compositions are provided in a dosage form for topical administration, which comprise a compound provided herein, and one or more pharmaceutically acceptable excipients or carriers.

The pharmaceutical compositions can also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (See, Remington: The Science and Practice of Pharmacy, supra; Modified-Release Drug Delivery Technology, 2nd Edition, Rathbone et al., Eds., Marcel Dekker, Inc.: New York, N.Y., 2008).

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In another embodiment, the pharmaceutical compositions provided herein further comprise one or more therapeutic agents for the treatment of Duchenne muscular dystrophy, Becker muscular dystrophy, or cachexia.

In yet another embodiment, provided herein is the use of a compound of formula (I) in the manufacture of a medicament for the treatment of Duchenne muscular dystrophy, Becker muscular dystrophy, or cachexia. In certain embodiments, the medicament is in tablet, capsule, powder, or liquid form. In certain embodiments, the medicament is formulated as described herein.

1. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms provided herein.

The pharmaceutical compositions provided herein for oral administration can be formulated as immediate or modified release dosage forms, including delayed-, sustained, pulsed-, controlled, targeted-, and programmed-release forms.

2. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (See, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

3. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (See, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

4. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

(a) Matrix Controlled Release Devices

The pharmaceutical compositions provided herein in a modified release dosage form can be fabricated using a matrix controlled release device known to those skilled in the art (See, Takada et al. in “Encyclopedia of Controlled Drug Delivery,” Vol. 2, Mathiowitz Ed., Wiley, 1999).

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

(b) Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art (See, Remington. The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

(c) Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

(d) Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### E. Methods of Use

Provided herein is a method for the treatment or prophylaxis of Duchenne muscular dystrophy, Becker muscular dystrophy, or cachexia, the method comprising administering to a patient in need thereof an effective amount of a compound of formula (I), or tautomer, enantiomer, pharmaceutically acceptable salt, hydrate, solvate, complex, or prodrog thereof.

In one embodiment, the dose of the compound of formula (I) is determined with consideration of age, body weight, general health condition, diet, administration time, administration method, clearance rate, combination of drugs, the level of disease for which the patient is under treatment for, and other factors. The dose varies depending on the target disease, condition, subject of administration, administration method and the like

In one embodiment, the pharmaceutical composition comprising a compound of formula (I) is administered orally as a therapeutic agent for the treatment of Duchenne muscular dystrophy in a patient suffering from such a disease. In one embodiment, about 0.01 mg to about 10 g of the compound is administered. In another embodiment, about 0.1 mg to about 100 mg of the compound is administered. In one embodiment, the compound is administered in a single dose. In another embodiment, the compound is administered in 2 or 3 portions per day.

In one embodiment, provided herein is a method for the treatment or prophylaxis of Duchenne muscular dystrophy or Becker muscular dystrophy, the method comprising administering to a patient in need thereof an effective amount of a compound of formula (I), or tautomer, enantiomer, pharmaceutically acceptable salt, hydrate, solvate, complex, or prodrog thereof.

In one embodiment, provided herein is a method of treating, preventing, and/or managing a disorder or symptoms related to Duchenne muscular dystrophy, Becker muscular dystrophy, or cachexia, the method comprising administering to a patient in need thereof an effective amount of a compound of formula (I), or tautomer, enantiomer, pharmaceutically acceptable salt, hydrate, solvate, complex, or prodrog thereof.

In another embodiment, provided herein is a method of treating, preventing, and/or managing a disorder or symptoms related to Duchenne muscular dystrophy or Becker muscular dystrophy, the method comprising administering to a patient in need thereof an effective amount of a compound of formula (I), or tautomer, enantiomer, pharmaceutically acceptable salt, hydrate, solvate, complex, or prodrog thereof.

In one embodiment, provided herein is a method of treating, preventing, and/or managing Duchenne muscular dystrophy. In another embodiment, provided herein is a method of treating, preventing, and/or managing Becker muscular dystrophy. In another embodiment, provided herein is a method of treating, preventing, and/or managing cachexia.

In one embodiment, provided herein is the use of a compound of formula (I), or tautomer, enantiomer, pharmaceutically acceptable salt, hydrate, solvate, complex, or prodrog thereof, in the manufacturing of a medicament for the treatment of Duchenne muscular dystrophy, Becker muscular dystrophy, or cachexia. In another embodiment, provided herein is the use of a compound of formula (I), or tautomer, enantiomer, pharmaceutically acceptable salt, hydrate, solvate, complex, or prodrog thereof, in the manufacturing of a medicament for the treatment of Duchenne muscular dystrophy or Becker muscular dystrophy. In another embodiment, provided herein is the use of a compound of formula (I), or tautomer, enantiomer, pharmaceutically acceptable salt, hydrate, solvate, complex, or prodrog thereof, in the manufacturing of a medicament for the treatment of Duchenne muscular dystrophy. In another embodiment, provided herein is the use of a compound of formula (I), or tautomer, enantiomer, pharmaceutically acceptable salt, hydrate, solvate, complex, or prodrog thereof, in the manufacturing of a medicament for the treatment of Becker muscular dystrophy. In another embodiment, provided herein is the use of a compound of formula (I), or tautomer, enantiomer, pharmaceutically acceptable salt, hydrate, solvate, complex, or prodrog thereof, in the manufacturing of a medicament for the treatment of cachexia.

In one embodiment, the method comprises administering to a subject (e.g., a human) a therapeutically or prophylactically effective amount of a composition of a compound of formula (I). In one embodiment, the subject is a human. In another embodiment, the subject is a mammal. In yet another embodiment, the subject is a non-human primate, a farm animal, such as cattle, a sport animal, or a pet such as a horse, dog, or cat.

In some embodiment, compound activity can be assessed by functional assays described herein elsewhere. In certain embodiments, the efficacious concentration of the compounds provided herein is less than 0.1 nM, less than 1 nM, less than 10 nM, less than 100 nM, less than 1 μM, less than 10 μM, less than 100 μM, or less than 1 mM. In other embodiments, compounds' activity may be assessed in various art-recognized animal models as described herein elsewhere.

In some embodiments, the compounds provided herein are active in at least one model, which can be used to measure the activity of the compounds and estimate their efficacy in treating Duchenne muscular dystrophy, Becker muscular dystrophy, or cachexia. For example, when the model is for Duchenne muscular dystrophy, the compounds are active in, for example the mdx mouse model, when compared to vehicle. In some embodiments, the compounds provided herein are active in a dose-dependent manner. In some embodiments, the compounds provided herein produce a similar disparity in measured endpoint between treated animals and animals treated with vehicle.

Depending on the disorder, disease, or condition to be treated, and the subject's condition, the compounds or pharmaceutical compositions provided herein can be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration and can be formulated, alone or together, in suitable dosage unit with pharmaceutically acceptable excipients, carriers, adjuvants, and vehicles appropriate for each route of administration. Also provided is administration of the compounds or pharmaceutical compositions provided herein in a depot formulation, in which the active ingredient is released over a predefined time period.

In the treatment, prevention, or amelioration of one or more symptoms of the disorders, diseases, or conditions described herein, an appropriate dosage level generally is ranging from about 0.001 to about 1000 mg per kg subject body weight per day (mg/kg per day), from about 0.001 to about 300 mg/kg per day, from about 0.001 to about 100 mg/kg per day, from about 0.01 to about 75 mg/kg per day, from about 0.1 to about 50 mg/kg per day, from about 0.5 to about 25 mg/kg per day, or from about 1 to about 20 mg/kg per day, which can be administered in single or multiple doses. Within this range, the dosage can be ranging from about 0.005 to about 0.05, from about 0.05 to about 0.5, from about 0.5 to about 5.0, from about 1 to about 15, from about 1 to about 20, or from about 1 to about 50 mg/kg per day.

In one embodiment, in the treatment, prevention, or amelioration of one or more symptoms of Duchenne muscular dystrophy, Becker muscular dystrophy, or cachexia, an appropriate dosage level is less than 0.001 mg/kg per day, less than 0.01 mg/kg per day, less than 0.1 mg/kg per day, less than 0.5 mg/kg per day, less than 1 mg/kg per day, less than 5 mg/kg per day, less than 10 mg/kg per day, less than 15 mg/kg per day, less than 20 mg/kg per day, less than 25 mg/kg per day, less than 50 mg/kg per day, less than 75 mg/kg per day, less than 100 mg/kg per day, less than 200 mg/kg per day, less than 500 mg/kg per day, or less than 1 g/kg per day.

For oral administration, the pharmaceutical compositions provided herein can be formulated in the form of tablets containing from about 1.0 to about 1,000 mg of the active ingredient, in one embodiment, about 1, about 5, about 10, about 15, about 20, about 25, about 50, about 75, about 100, about 150, about 200, about 250, about 300, about 400, about 500, about 600, about 750, about 800, about 900, and about 1,000 mg of the active ingredient for the symptomatic adjustment of the dosage to the patient to be treated. The pharmaceutical compositions can be administered on a regimen of 1 to 4 times per day, including once, twice, three times, and four times per day.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In another embodiment, the compounds provided herein, e.g., a compound of formula (I), or tautomer, enantiomer, pharmaceutically acceptable salt, hydrate, solvate, complex, or prodrog thereof, can be combined or used in combination with other agents or therapies useful in the treatment, prevention, or amelioration of Duchenne muscular dystrophy, Becker muscular dystrophy, or cachexia. Suitable other therapeutic agents include, but are not limited to, corticosteroids, such as for example, prednisone and deflazacort. In certain embodiments, the other therapies that may be used in combination with the compounds provided herein include, but are not limited to, physical therapy, gene therapy, or orthopedic appliances, such as braces and wheelchairs.

Such other agents, or drugs, can be administered, by a route and in an amount commonly used therefor, simultaneously or sequentially with the compounds provided herein, or tautomer, enantiomer, pharmaceutically acceptable salt, hydrate, solvate, complex, or prodrog thereof. When a compound provided herein is used contemporaneously with one or more other drugs, a pharmaceutical composition containing such other drugs in addition to the compound provided herein can be utilized, but is not required. Accordingly, the pharmaceutical compositions provided herein include those that also contain one or more other active ingredients or therapeutic agents, in addition to a compound provided herein.

The weight ratio of a compound provided herein to the second active ingredient can be varied, and will depend upon the effective dose of each ingredient. Generally, an effective dose of each will be used. Thus, for example, when a compound provided herein is combined with a corticosteroid, the weight ratio of the compound to the corticosteroid can range from about 1,000:1 to about 1:1,000, about 200:1 to about 1:200, about 100:1 to about 1:100, or about 10:1 to about 1:10. Combinations of a compound provided herein and other active ingredients will generally also be within the aforementioned range, but in each case, an effective dose of each active ingredient should be used.

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a compound provided herein.

In certain embodiments, the kit includes a container comprising a dosage form of the compound provided herein, or tautomer, enantiomer, pharmaceutically acceptable salt, hydrate, solvate, complex, or prodrog thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### EXAMPLES

Certain embodiments are illustrated by the following non-limiting examples.

HPLC-UV-MS was performed on a Gilson 321 HPLC with detection performed by a Gilson 170 DAD and a Finnigan AQA mass spectrometer operating in electrospray ionisation mode. The HPLC column used is a Phenomenex Gemini C18 150×4.6 mm.

In Examples 1 to 15, preparative HPLC was performed on a Gilson 321 with detection performed by a Gilson 170 DAD. Fractions were collected using a Gilson 215 fraction collector. The preparative HPLC column used is a Phenomenex Gemini C18 150×10 mm and the mobile phase is acetonitrile/water.

In Examples 16 to 21, preparative HPLC was performed on a Dionex Ultimate 3000 system incorporating a Foxy Jr. fraction collector. The preparative scale column is a Phenomenex Gemini C18 100×30 mm, with the mobile phase consisting of 0.1% formic acid in water and 0.1% formic acid in acetonitrile.

¹H NMR spectra were recorded on a Bruker instrument operating at 300 MHz.

NMR spectra were obtained as CDCl₃ solutions (reported in ppm), using chloroform as the reference standard (7.25 ppm) or DMSO-D₆ (2.50 ppm). When peak multiplicities are reported, the following abbreviations are used s (singlet), d (doublet), t (triplet), m (multiplet), br (broadened), dd (doublet of doublets), dt (doublet of triplets), td (triplet of doublets). Coupling constants, when given, are reported in Hertz (Hz).

Column chromatography was performed either by flash chromatography (40-65 μm silica gel) or using an automated purification system (SP1™ Purification System from Biotage® or an ISCO Companion). Reactions in the microwave were done in an Initiator 8™ (Biotage) or an Explorer 48 (CEM).

The abbreviations used are DMSO (dimethylsulfoxide), HATU (O-(7-azabenzotriazol-1yl)-N,N,N′,N′-tetramethyluronium hexafluorophosphate), HCl (hydrochloric acid), MgSO₄ (magnesium sulfate), NaOH (sodium hydroxide), Na₂CO₃ (sodium carbonate), NaHCO₃ (sodium bicarbonate), STAB (sodium triacetoxyborohydride), THF (tetrahydrofuran).

### Example 1

### Preparation of N-(benzo[d][1,3]-dioxol-5-yl)-1-(4-(methylsulfonyl)benzyl)-1H-indazole-3-carboxamide (Compound 4)

### Methyl 2H-indazole-3-carboxylate

Procedure A: A solution of 2H-indazole-3-carboxylic acid (5.00 g, 30.8 mmol) and concentrated sulphuric acid (0.16 mL, 3.08 mmol) in methanol (100 mL) was heated to reflux for 16 hours. The reaction mixture was concentrated in vacuo and then partitioned between ethyl acetate and water, washed with saturated sodium bicarbonate solution (aq.), the aqueous phase extracted with ethyl acetate, the combined organic layers washed with brine, dried (magnesium sulfate) and concentrated to give 2.02 g (93%) of the title compound.

### Methyl 1-(4-(methylsulfonyl)benzyl)-1H-indazole-3-carboxylate &

### Methyl 2-(4-(methylsulfonyl)benzyl)-2H-indazole-3-carboxylate

Procedure B: A solution of 1H-indazole-3-carboxylate (200 mg, 1.14 mmol) with 1-(chloromethyl)-4-(methylsulfonyl)benzene (233 mg, 1.14 mmol) and potassium carbonate (0.47 g, 3.41 mmol) in N,N-dimethylformamide (1.5 mL) was microwaved for 10 min at 130° C. The reaction mixture was partitioned between ethyl acetate and water, the organic phase washed with water (2×), the aqueous phase extracted with ethyl acetate (1×) and the combined organic phases washed with brine, dried (magnesium sulfate) and concentrated in vacuo. The residue was purified by column chromatography eluting using a gradient (petroleum ether 40/60/ethyl acetate 1:0 v/v 2:8) to afford 216 mg (55%) of methyl 1-(4-(methylsulfonyl)benzyl)-1H-indazole-3-carboxylate, ¹H NMR (CDCl₃): 8.21 (1H, d, J 8.1), 7.82 (2H, d, J 8.4), 7.39-7.27 (5H, m), 5.76 (2H,$), 4.024 (3H, s), 2.97 (3H, s), and 101 mg (25%) of methyl 2-(4-(methylsulfonyl)benzyl)-2H-indazole-3-carboxylate ¹H NMR (CDCl₃): 7.93 (1H, dt, J 8.3 1.0), 7.80-7.72 (3H, m), 7.40 (2H, d, J 8.6), 7.33-7.20 (2H, m), 6.10 (2H, s), 3.92 (3H, s), 2.91 (3H, s).

### 1-(4-(Methylsulfonyl)benzyl)-1H-indazole-3-carboxylic acid

Procedure C: A solution of methyl 1-(4-(methylsulfonyl)benzyl)-1H-indazole-3-carboxylate (1.96 g, 5.7 mmol) and sodium hydroxide (6.84 mL 1M solution, 6.84 mmol) in dioxane (30 mL) was stirred at room temperature. The reaction mixture was neutralized with Amberlite-H⁺, filtered and concentrated under reduced pressure to afford the title compound in quantitative yield, which was used in the next step without further purification.

### N-(Benzo[d][1,3]dioxol-5-yl)-1-(4-(methylsulfonyl)benzyl)-1H-indazole-3-carboxamide (Compound 4)

Procedure D: 1-(4-(Methylsulfonyl)benzyl)-1H-indazole-3-carboxylic acid (300 mg, 0.91 mmol) was taken up in N,N-dimethylformamide (5 mL), benzo[d][1,3]dioxol-5-amine (150 mg, 1.09 mmol), (benzotriazol-1-yloxy)tripyrrolidinophosphonium hexafluorophosphate (567 mg, 1.09 mmol) and diisopropylethylamine (0.47 mL, 2.73 mmol) were added and the resulting mixture was stirred at room temperature for 16 h. The reaction mixture was partitioned between ethyl acetate and water, the organic phase washed with water (2×), the aqueous phase extracted with ethyl acetate (1×) and the combined organic phases washed with brine, dried over magnesium sulfate and concentrated. The residue was purified by column chromatography eluting using a gradient (petroleum ether 40/60/ethyl acetate 1:0 v/v 3:7) followed by recrystallisation with petroleum ether 40/60/ethyl acetate to afford 317 mg (78%). (LCMS RT=6.03 min, MH⁺=450.2), ¹H NMR (CDCl₃): 8.73 (1H, s), 8.49 (1H, d, J 8.0), 7.93 (2H, d, J 8.5), 7.50-7.44 (2H, m), 7.40-7.36 (4H, m), 7.05 (1H, d, J 8.3 2.2), 6.82 (1H, d, J 8.3), 6.00 (2H, s), 5.75 (2H, s), 3.04 (3H, s).

The following compounds have been prepared and worked-up by the procedure D described above. Purification of the products to required purity specifications has been carried out by column chromatography and/or trituation (s)/recrystalisation(s).

### N-(3,4-Dimethoxyphenyl)-1-(4-(methylsulfonyl)benzyl)-1H-indazole-3-carboxamide (Compound 82)

The title compound was prepared according to procedure D to afford 272 mg (99%). (LCMS RT=5.87 min, MH⁺=466.2), ¹H NMR (CDCl₃): 8.76 (1H, s), 8.53-8.48 (1H, br m), 7.93 (2H, d, J 8.3), 7.64 (1H, d, J 2.4), 7.52-7.34 (5H, br m), 7.11 (1H, dd, J 8.6 and 2.5), 6.94-6.85 (1H, br m), 5.76 (2H, s), 3.98 (3H, s), 3.92 (3H, s), 3.05 (3H, s).

### N-(3-methoxyphenyl)-1-(4-(methylsulfonyl)benzyl)-1H-indazole-3-carboxamide (Compound 86)

The title compound was prepared according to procedure D to afford 56 mg (88%). (LCMS RT=6.3 min, MH⁺=436), ¹H NMR (CDCl₃): 8.76 (1H, s), 8.39 (1H, d, J 8.4), 7.80 (2H, d, J 8.4), 7.46 (1H, t, J 2.1), 7.39-7.31 (1H, br m), 7.30-7.09 (6H, br m), 6.65-6.59 (1H, br m), 5.63 (2H, s), 3.76 (3H, s), 2.92 (3H, s).

### N-(4-hydroxyphenyl)-1-(4-(methylsulfonyl)benzyl)-1H-indazole-3-carboxamide (Compound 88)

The title compound was prepared according to procedure D to afford 64 mg (51%). (LCMS RT=5.52 min, MH⁺=422.2), ¹H NMR (CDCl₃): 8.52 (1H, br s), 8.29 (1H, br m), 7.72 (2H, d, J 8.5), 7.41 (2H, d, J 8.9), 7.30-7.22 (1H, br m), 7.22-7.12 (4H, br m), 6.73-6.63 (1H, br m) 5.54 (2H, s), 2.83 (3H, s).

### N-(4-Isopropylphenyl)-1-(4-(methylsulfonyl)benzyl)-1H-indazole-3-carboxamide (Compound 62)

The title compound was prepared according to procedure D to afford 154 mg (56%). (LCMS RT=7.02 min, MH⁺=448.5), ¹H NMR (CDCl₃): 8.78 (1H, s), 8.51 (1H, d, J 8.0), 7.92 (2H, d, J 8.4), 7.68 (2H, d, J 8.5) 7.46-7.34 (5H, m), 7.26 (2H, m), 5.75 (2H, s), 5.75 (2H, s), 3.04 (3H, s), 2.92 (1H, m), 1.29 (3H, s), 1.27 (3H, s).

### N-(4-Ethylphenyl)-1-(4-(methylsulfonyl)benzyl)-1H-indazole-3-carboxamide (Compound 84)

The title compound was prepared according to procedure D to afford 244 mg (92%). (LCMS RT=6.71 min, MH⁺=434.2), ¹H NMR (CDCl₃): 8.79 (1H, s), 8.54-8.49 (1H, br m), 7.94 (2H, d, J 8.4), 7.68 (2H, d, J 8.5), 7.50-7.34 (5H, br m), 7.28-7.21 (2H, br m—overlapping with NMR solvent signal at left hand side), 5.76 (2H, s), 3.04 (3H, s), 2.67 (2H, q, J 7.6), 1.31-1.23 (3H, s).

### 1-(4-(Methylsulfonyl)benzyl)-N-p-tolyl-1H-indazole-3-carboxamide (Compound 108)

The title compound was prepared according to procedure D to afford 124 mg (88%). (LCMS RT=2.46 min, MH⁺=420.1), ¹H NMR (CDCl₃): 6.68 (1H, br s), 8.40 (1H, d, J 8.6), 7.82 (2H, d, J 8.3), 7.55 (2H, d, J 8.2), 7.40-7.22 (6H, br m), 7.11 (1H, d, J 8.1), 5.65 (2H, s), 2.94 (3H, s), 2.27 (3H, s).

### N-(3,4-Dimethylphenyl)-1-(4-(methylsulfonyl)benzyl)-1H-indazole-3-carboxamide (Compound 104)

The title compound was prepared according to procedure D to afford 125 mg (93%). (LCMS RT=2.53 min, MH⁺=434.3), ¹H NMR (DMSO-d6): 10.16 (1H, br s), 8.25 (1H, d, J 8.2), 8.36 (2H, d, J 8.4), 7.84 (1H, d, J 8.6), 7.70-7.67 (1H, br m), 7.60-7.55 (1H, d, J 8.0), 7.55-7.47 (3H, br, m), 7.34 (1H, t, J 7.6), 7.10 (1H, d, J 8.4), 5.95 (2H, s), 3.17 (3H, s), 2.23 (3H, s), 2.20 (3H, s).

### 1-(4-(Methylsulfonyl)benzyl)-N-(4-propylphenyl)-1H-indazole-3-carboxamide (Compound 112)

The title compound was prepared according to procedure D to afford 95 mg (70%). LCMS: RT 2.74 min, MH⁺448.1 ¹H NMR (DMSO-d6): 10.28 (1H, s), 8.26 (1H, d, J 8.2), 7.92-7.74 (5H, br m), 7.55-7.46 (3H, br m), 7.34 (1H, t, J 7.4), 7.17 (2H, t, J 8.4), 5.96 (2H, s), 3.16 (3H, s), 2.57 (2H, br m—obscured by NMR solvent signal on right hand side), 1.66-1.52 (2H, br m), 0.90 (3H, t, J).

### N-(4-Tert-butylphenyl)-1-(4-(methylsulfonyl)benzyl)-1H-indazole-3-carboxamide (Compound 81)

The title compound was prepared according to procedure D to afford 207 mg (99%). (LCMS RT=7.40 min, MH⁺=462.1), ¹H NMR (CDCl₃): 8.79 (1H, s), 8.54-8.48 (1H, br m), 7.94 (2H, d, J 8.5), 7.68 (2H, d, J 8.7), 7.50-7.34 (7H, br m), 5.76 (2H, s), 3.04 (3H, s), 1.35 (9H, s).

### N-(2-Isopropylphenyl)-1-(4-(methylsulfonyl)benzyl)-1H-indazole-3-carboxamide (Compound 85)

The title compound was prepared according to procedure D to afford 248 mg (91%). (LCMS RT=6.66 min, MH⁺=448.4), ¹H NMR (CDCl₃): 8.89 (1H, s), 8.53-8.48 (1H, br m), 8.12 (1H, dd, J 8.0 and 1.5), 7.94 (2H, d, J 8.5), 7.51-7.27 (7H, br m—right hand side overlapping with NMR solvent signal), 7.21 (1H, td, J 7.5 and 1.6), 5.77 (2H, s), 3.28-3.14 (1H, br m), 3.04 (3H, s), 1.34 (6H, d, J 6.8).

### N-(3-Isopropylphenyl)-1-(4-(methylsulfonyl)benzyl)-1H-indazole-3-carboxamide (Compound 96)

The title compound was prepared according to procedure D to afford 120 mg (89%). (LCMS RT=6.86 min, MH⁺=448.2), ¹H NMR (CDCl₃): 8.82 (1H, br s), 8.52 (1H, d, J 8.4), 7.93 (2H, d, J 8.4), 7.65 (1H, s), 7.59 (1H, d, J 8.3), 7.51-7.29 (6H, br m), 7.05 (1H, d, J 7.7), 5.76 (2H, s), 3.07-2.91 (4H, s and br m overlapping), 1.31 (6H, d, J 6.9).

### N-Phenyl-1-(4-(methylsulfonyl)benzyl)-1H-indazole-3-carboxamide (Compound 83)

The title compound was prepared according to procedure D to afford 181 mg (78%). (LCMS RT=6.22 min, MH⁺=406.1), ¹H NMR (CDCl₃): 8.84 (1H, s), 8.54-8.48 (1H, br m), 7.93 (2H, d, J 8.3), 7.77 (2H, d, J 8.1), 7.51-7.34 (7H, br m), 7.17 (1H, t, J 7.4), 5.76 (2H, s), 3.04 (3H, s).

### 1-(4-(Methylsulfonyl)benzyl)-N-(4-(piperidin-1-yl)phenyl)-1H-indazole-3-carboxamide (Compound 18)

The title compound was prepared according to procedure D to afford 431 mg (58%). (LCMS RT=4.62 min, MH⁺=489.3), ¹H NMR (CDCl₃): 8.70 (1H, s), 8.54-8.48 (1H, br m), 7.93 (2H, d, J 8.4), 7.63 (2H, d, J 8.9), 7.50-7.32 (5H, br m), 6.99 (2H, d, J 9.0), 5.75 (2H, s), 3.19-3.12 (4H, br m), 3.04 (3H, s), 1.79-1.70 (4H, br m), 1.64-1.58 (2H, br m—overlapping with water signal at right hand side).

### N-(4-Chlorophenyl)-1-(4-(methylsulfonyl)benzyl)-1H-indazole-3-carboxamide (Compound 89)

The title compound was prepared according to procedure D to afford 101 mg (77%). (LCMS RT=6.69 min, MH⁺=439.9), ¹H NMR (CDCl₃): 8.83 (1H, br s), 8.48 (1H, d, J 8.5), 7.93 (2H, d, J 8.3), 7.73 (2H, d, J 8.9), 7.52-7.44 (1H, br m), 7.43-7.33 (6H, br m), 5.76 (2H, s), 3.04 (3H, s).

### N-(3,4-Dichlorophenyl)-1-(4-(methylsulfonyl)benzyl)-1H-indazole-3-carboxamide (Compound III)

The title compound was prepared according to procedure D to afford 87 mg (61%). LCMS: RT 2.81 min, MH⁺not found ¹H NMR (DMSO-d6): 10.74 (1H, s), 8.32-8.23 (2H, br m), 7.95-7.82 (4H, br m), 7.62 (1H, d, J 8.8), 7.56-7.47 (3H, br m), 7.36 (1H, t, J 7.4), 5.98 (2H, s), 3.17 (3H, s).

### N-(4-Fluorophenyl)-1-(4-(methylsulfonyl)benzyl)-1H-indazole-3-carboxamide (Compound 94)

The title compound was prepared according to procedure D to afford 117 mg (92%). (LCMS RT=6.14 min, MH⁺=424.1), ¹H NMR (DMSO-d6): 10.49 (1H, br m), 8.26 (1H, d, J 8.1), 7.95-7.81 (5H, br m), 7.53-7.49 (3H, br m), 7.35 (1H, t, J 7.5), 7.20 (2H, t, J 8.8), 5.97 (2H, s), 3.17 (3H, s).

### N-(4-Acetamidophenyl)-1-(4-(methylsulfonyl)benzyl)-1H-indazole-3-carboxamide (Compound 107)

The title compound was prepared according to procedure D to afford 60 mg (43%). (LCMS RT=1.94 min, MH⁺=463.2), ¹H NMR (CD₃OD): 8.23 (1H, d, J 8.3), 7.82 (2H, d, J 8.05), 7.63 (2H, d, J 8.8), 7.57-7.32 (6H, Y, J), 7.24 (1H, t, J 7.5), 5.82 (2H, s), 4.50 (1H, br s), 2.98 (3H, s), 2.04 (3H, s).

### N-(3-Isopropyl-1,2,4-thiadiazol-5-yl)-1-(4-(methylsulfonyl)benzyl)-1H-indazole-3-carboxamide (Compound 98)

The title compound was prepared according to procedure D to afford 69 mg (39%). (LCMS RT=6.35 min, MH⁺=455.9), ¹H NMR (CDCl₃): 8.42 (1H, br s), 7.94 (2H, d, J 8.3), 7.57-7.40 (5H, br m), 5.75 (2H, s), 3.33-3.17 (1H, br m), 3.05 (3H, s), 1.41 (6H, d, J 7.0).

### 1-(4-(Methylsulfonyl)benzyl)-N-(quinolin-6-yl)-1H-indazole-3-carboxamide (Compound 99)

The title compound was prepared according to procedure D to afford 64 mg (47%). (LCMS RT=4.82 min, MH⁺=457.1), ¹H NMR (DMSO-d6): 10.75 (1H, br s), 8.81 (1H, dd, J4.3 and 1.6), 8.67 (1H, d, J 2.3), 8.32 (2H, t, J 8.3), 8.18 (1H, dd J 9.1 and 2.4), 8.00 (1H, d, J 9.2), 7.94-7.84 (3H, br m), 7.57-7.48 (4H, br m), 7.38 (1H, t, J 7.6), 6.00 (2H, s), 3.17 (3H, s).

### 1-(4-(Methylsulfonyl)benzyl)-N-(quinolin-3-yl)-1H-indazole-3-carboxamide (Compound 103)

The title compound was prepared according to procedure D to afford 27 mg (20%). (LCMS RT=2.29 min, MH⁺=457.1), ¹H NMR (DMSO-d6): 10.93 (1H, br s), 9.29-9.25 (1H, br m), 8.98-8.93 (1H, br m), 8.31 (1H, d, J 8.0), 8.02-7.84 (5H, br m), 7.73-7.46 (5H, br m), 7.39 (1H, t, J 7.3), 6.01 (2H, s), 3.17 (3H, s).

### N-(2,3-Dihydrobenzo[b][1,4]dioxin-6-yl)-1-(4-(methylsulfonyl)benzyl)-1H-indazole-3-carboxamide (Compound 105)

The title compound was prepared according to procedure D to afford 146 mg (90%). LCMS RT=2.26 min, MH⁺=464), ¹H NMR (CDCl₃): 8.44 (1H, br s), 8.28-8.23 (1H, br m), 7.69 (2H, d, J 8.5), 7.26-7.09 (6H, br m), 6.92 (1H, dd, J 8.7 and 2.5), 6.64 (1H, d, J 8.7), 5.51 (2H, s), 4.08-4.01 (4H, br m), 2.80 (3H, s).

### 1-(4-(Methylsulfonyl)benzyl)-N-(quinoxalin-6-yl)-1H-indazole-3-carboxamide (Compound 110)

The title compound was prepared according to procedure D to afford 58 mg (42%). LCMS: RT 2.08 min, MH⁺458.1 ¹H NMR (DMSO-d6): 10.97 (1H, br s), 8.92 (1H, d, J 1.9), 8.85 (1H, d, J 1.8), 8.80 (1H, d, J 2.2), 8.39-8.30 (2H, br m), 8.09 (1H, d, J 9.1), 7.94-7.85 (3H, br m), 7.58-7.50 (3H, br m), 7.40 (1H, t, J 7.6), 6.02 (2H, s), 3.17 (3H, s).

### N-(1,3-Dihydrobenzo[c]thiophen-2,2-dioxy-5-yl)-1-(4-(methylsulfonyl)benzyl)-1H-indazole-3-carboxamide (Compound 113)

The title compound was prepared according to procedure D to afford 83 mg (56%). LCMS: RT 2.09 min, MH⁺ not found ¹H NMR (DMSO-d6): 10.54 (1H, s), 8.26 (1H, d, J 8.2), 8.05-8.02 (1H, br m), 7.93-7.67 (4H, br m), 7.55-7.48 (3H, br m), 7.39-7.32 (2H, br m), 5.98 (2H, s), 4.53 (2H, s), 4.46 (2H, s), 3.17 (3H, s).

### N-(Benzo[d][1,3]dioxol-5-yl)-1-(isoxazol-3-ylmethyl)-1H-indazole-3-carboxamide (Compound 145)

The above compound was synthesised according to procedures C-D, starting from methyl 1-(isoxazol-3-ylmethyl)-1H-indazole-3-carboxylate, to afford 30 mg (100% step C, 41% step D). LCMS RT=2.32 min, MH⁺=363.0 ¹H NMR (DMSO): 10.30 (1H, s), 8.90 (1H, d, J 1.5 Hz), 8.23 (1H, d, J 8.1 Hz), 7.83 (1H, d, J 8.5 Hz), 7.55-7.49 (2H, m), 7.37-7.32 (2H, m), 6.90 (1H, d, J 8.4 Hz), 6.52 (1H, d, J 1.6 Hz), 6.01 (2H, s), 5.96 (2H, s).

### N-(Benzo[d][1,3]dioxol-5-yl)-1-(naphthalen-2-ylmethyl)-1H-indazole-3-carboxamide (Compound 141)

The title compound was prepared according to procedure D to afford 92 mg (23%). (LCMS RT=2.97 min, MH⁺=422.4), ¹H NMR (CHCl₃): 8.72 (1H, s), 8.38 (1H, d, J 9.4), 7.75-7.68 (3H, m), 7.56 (1H, s), 7.43-7.22 (7H, m), 6.97 (1H, dd, J 2.5, 9.7), 6.72 (1H, d, J 6.7), 5.89 (2H, s), 5.72 (2H, s).

### N-(Benzo[d][1,3]dioxol-5-yl)-1-(benzo[d][1,3]dioxol-5-ylmethyl)-1H-indazole-3-carboxamide (Compound 123)

The title compound was prepared according to the procedure D to afford 24 mg (22%). (LCMS RT=2.61 min, MH⁺=416.3), ¹H NMR (CDCl₃): 8.78 (1H, s), 8.45 (1H, d, J 9.5), 7.51 (1H, d, J 2.5), 7.42 (2H, m), 7.35-7.30 (1H, m), 7.07 (1H, dd, J 2.5, 9.7), 6.82 (1H, d, J 9.7), 6.77 (2H, m), 6.71 (1H, br s), 6.00 (2H, s), 5.95 (2H, s), 5.56 (2H, s).

### N-(2,2-Difluorobenzo[d][1,3]dioxol-5-yl)-1-(4-(dimethylcarbamoyl)benzyl)-1H-indazole-3-carboxamide (Compound 135)

The title compound was prepared according to procedure D to afford 100 mg (22%). (LCMS RT=2.42 min, MH⁺=479.2, 956.9), ¹H NMR (CDCl₃): 8.50 (1H, s), 7.73 (1H, d, J 10.0), 7.66 (1H, d, J 9.8), 7.62 (1H, d, J 1.5), 7.28 (1H, t, 18.1), 7.18 (3H, m), 7.03 (3H, m), 6.93 (1H, d, J 9.9), 5.92 (2H, s), 2.90 (3H, s), 2.76 (3H, s).

### 1-(4-(Dimethylcarbamoyl)benzyl)-N-p-tolyl-1H-indazole-3-carboxamide (Compound 138)

The title compound was prepared according to procedure D to afford 111 mg (57%). (LCMS RT=2.44 min, MH⁺=413.2, 825.6), ¹H NMR (CDCl₃): 8.82 (1H, s), 8.48 (1H, d, J 9.5), 7.66 (1H, d, J 9.8), 7.47-7.31 (5H, m), 7.26 (1H, s), 7.23 (2H, d, J 4.0), 7.19 (1H, s), 5.69 (2H, s), 3.12 (3H, s), 2.97 (3H, s), 2.37 (3H, s).

### 1-(4-(Dimethylcarbamoyl)benzyl)-N-(4-isopropylphenyl)-1H-indazole-3-carboxamide (Compound 139)

The title compound was prepared according to procedure D to afford 151 mg (37%). (LCMS RT=2.66 min, MH⁺=441.0, 882.0), ¹H NMR (CDCl₃): 9.95 (1H, s), 8.27 (1H, d, J 9.5), 7.79-7.75 (3H, m), 7.51-7.46 (1H, m), 7.39-7.30 (5H, m), 7.23 (1H, d, J 9.9), 5.85 (2H, s), 3.11 (6H, s), 1.23 (7H, d, J 8.1).

### Example 2

### Preparation of N-(Benzo[d][1,3]-dioxol-5-yl)-1-(4-tert-butylbenzoyl-1)-1H-indazole-3-carboxamide (Compound 124)

### N-(Benzo[d][1,3]dioxol-5-yl)-1-(4-tert-butylbenzoyl)-1H-indazole-3-carboxamide (Compound 124)

The title compound was prepared according to the protocol D, then N-(benzo[d][1,3]dioxol-5-yl)-1H-indazole-3-carboxamide (350 mg, 1.3 mmol) was dissolved in dry DMF (5 ml), and NaH (55 mg, 1.4 mmol) was added. The mixture was cooled to 0° C., stirred for 1 hour, and 4-isopropylbenzoyl chloride (0.25 ml, 1.4 mmol) was added. The mixture was warmed to room temperature and left stirring for 18 hours. The reaction mixture was partitioned between ethyl acetate and water, washed with 1M HCl solution (1×), saturated sodium bicarbonate solution (aq.), the aqueous phase extracted with ethyl acetate, the combined organic layers washed with brine, dried (magnesium sulfate) then concentrated. The compound was purified by column chromatography with gradient (petroleum ether 40/60/ethyl acetate 1:0 v/v 8:2) followed by trituration with methanol to give 270 mg (49%) of the title compound. (LCMS RT=3.51 min, MH⁺=442.1), ¹H NMR (DMSO): 10.48 (1H, s), 8.48, (1H, d, J 9.9), 8.26 (1H, d, J 9.3), 8.15 (2H, d, J 9.8), 7.77 (1H, t, J 8.4), 7.64 (2H, d, J 9.9), 7.58 (1H, t, J 9.3), 7.45 (1H, s), 7.23 (1H, dd, J 2.3, 9.8), 6.93 (1H, d, J 9.8), 6.03 (2H, s), 1.35 (9H, s)

This compound's regiochemistry was confirmed by X-ray crystallography.

### Example 3

### Preparation of N-(Benzo[d][1,3]-dioxol-5-yl)-1-(4-(methylsulfonyl)benzoyl)-1H-indazole-3-carboxamide (Compound 140)

N-(benzo[d][1,3]dioxol-5-yl)-1H-indazole-3-carboxamide (300 mg, 1.1 mmol) was dissolved in DMF (5 mL), 4-(methylsulfonyl)benzoic acid (260 mg, 1.2 mmol), (benzotriazol-1-yloxy)tripyrrolidinophosphonium hexafluorophosphate (690 mg, 1.2 mmol) and diisopropylethylamine (0.57 mL, 3.3 mmol) were added and the resulting mixture was stirred at room temperature for 16 h. The reaction mixture was partitioned between ethyl acetate and water, the organic phase washed with water (2×), the aqueous phase extracted with ethyl acetate (1×) and the combined organic phases washed with brine, dried over magnesium sulfate and concentrated. The residue was purified by column chromatography eluting using a gradient (petroleum ether 40/60/ethyl acetate 1:0 v/v 9:1) to afford 51 mg (10%) of the title compound.

### N-(Benzo[d][1,3]dioxol-5-yl)-1-(4-(methylsulfonyl)benzoyl)-1H-indazole-3-carboxamide

(LCMS RT=2.49 min, MH⁺=463.8, 927.3), ¹H NMR (DMSO): 10.92 (1H, s), 8.52 (1H, d, J 9.8), 8.38 (2H, d, J 9.8), 8.29 (1H, d, J 9.3), 8.14 (2H, d, J 9.8), 7.81 (1H, t, J 8.6), 7.62 (1H, t, J 8.6), 7.44 (1H, d, J 2.3), 7.21 (1H, dd, J 2.4, 7.8), 6.93 (1H, d, J 9.8), 6.03 (2H, s).

### Example 4

### Preparation of N-(Benzo[d][1,3]-dioxol-5-yl)-1-(4-(methylsulfonyl)benzyl)-1H-indole-3-carboxamide (Compound 146)

### Methyl 1H-pyrazole-3-carboxylate

The title compound was prepared following procedure A (from 1H-pyrazole-3-carboxylic acid) to afford 1.514 g (68%) of the title compound. ¹H NMR (CDCl₃): 7.88 (1H, d, J 2.4), 6.87 (1H, d, J 2.3), 3.98 (3H, s).

### Methyl 1-(4-(methylsulfonyl)benzyl)-1H-indole-3-carboxylate

This compound was prepared according to procedure B.

### 1-(4-(Methylsulfonyl)benzyl)-1H-indole-3-carboxylic acid

A solution of methyl 1-(4-(methylsulfonyl)benzyl)-1H-indole-3-carboxylate (300 mg, 0.87 mmol, 1 eq) in THF/H₂O (3.5 mL, 1/1) and NaOH (1N, V=7 mL) was stirred at room temperature for 18 hours. Then an extra 7 ml of NaOH 2N was added to the mixture and was refluxed for 16 hours. HCl 2N was added until pH=2, reaction mixture was put in cold ice until a white solid crashed out. The solid was filtered and dried in vacuo to give 200 mg as a white solid (69%). ¹H NMR (DMSO): 8.35 (1H, s), 8.08 (m, 1H, J 3.2 Hz), 7.94 (d, 2H, J 8.3 Hz), 7.56 (m, 3H, J 7.9 Hz), 7.25 (m, 2H, J 1.8 Hz), 5.70 (s, 2H), 3.22 (s, 3H).

### N-(Benzo[d][1,3]dioxol-5-yl)-1-(4-(methylsulfonyl)benzyl)-1H-indole-3-carboxamide

Procedure B: 1 1-(4-(methylsulfonyl)benzyl)-1H-indole-3-carboxylic acid (100 mg, 0.30 mmol) was taken up in DCM (2.5 mL) with drops of DMF because of poor solubility, 3,4 (methylendioxy)aniline (63 mg, 1.5 eq), benzotriazol-1-yloxytris(dimethylamino)phosphonium hexafluorophosphate (200 mg, 1.5 eq) and diisopropylethylamine (0.08 mL, 1.5 eq) were added and the resulting mixture was stirred at room temperature for 16 h. The reaction mixture was partitioned between DCM and water, the organic phase washed with water (2×), the aqueous phase extracted with DCM (1×) and the combined organic phases washed with brine, dried over magnesium sulfate and concentrated. The residue was purified by column chromatography eluting using a gradient (petroleum ether 30/70/ethyl acetate 1:0 v/v 3:7) followed by recrystallisation with methanol to afford 50 mg of the title compound as a white solid. LCMS RT=2.24 min, MH⁺=449.1, 100% UV purity. ¹H NMR (DMSO): 9.74 (1H, s), 8.36 (1H, s), 8.25 (1H, s), 8.20 (1H, dd, J 2.73 Hz), 7.90 (2H, d, J 8.3 Hz), 7.53 (2H, m, J 6.99 Hz), 7.47 (2H, m, J 5.32 Hz), 7.19 (2H, m, J 4.79 Hz), 7.12 (1H, dd, J 3.46 Hz), 6.88 (1H, d, J 8.4 Hz), 5.99 (2H, s), 5.66 (1H,$), 3.20 (3H, s).

The following compounds were prepared by an analogous method.

### 1-(4-(Methylsulfonyl)benzyl)-N-(5-methylthiazol-2-yl)-1H-indazole-3-carboxamide (Compound 125)

The above compound was synthesised according to procedure B. LCMS RT=2.33 min, MH⁺=427.1, 100% UV purity. ¹H NMR (DMSO): 12.13 (1H, s), 8.21 (1H, d, J 7.98 Hz), 7.89 (3H, m, J 9.96 Hz), 7.65 (2H, d, J 8.25 Hz), 7.52 (1H, t, J 7.68 Hz), 7.36 (1H, t, J 87.51 Hz), 7.22 (1H, s), 5.93 (2H, s) 3.16 (3H, s), 2.39 (3H, s).

### 1-(4-(methylsulfonyl)benzyl)-N-(4-methylthiazol-2-yl)-1H-indazole-3-carboxamide (Compound 126)

The above compound was synthesised according to procedure B. LCMS RT=2.33 min, MH⁺=427.1, 90% UV purity. 1H NMR (DMSO): 8.21 (1H, t, J 7.87 Hz), 7.90 (3H, m, J 8.15 Hz), 7.70 (2H, d, J 8.31 Hz), 7.50 (1H, m, J 8.26 Hz), 7.22 (1H, t, J 7.50 Hz), 6.85 (1H, s), 5.92 (2H, s), 3.16 (3H, s), 2.31 (3H, s).

### N-(5-Methylpyridin-2-yl)-1-(4-(methylsulfonyl)benzyl)-1H-indazole-3-carboxamide (Compound 134)

The above compound was synthesised according to procedure B. LCMS RT=2.21 min, MH⁺=421.1.1, 100% UV purity. ¹H NMR (DMSO): 9.77 (1H, s), 8.20 (3H, m, J 8.61 Hz), 7.8 ppm (3H, d, J 8.35 Hz), 7.70 (1H, dd, J 3.50 Hz), 7.56 (3H, m, J 8.20 Hz), 7.37 (1H, t, J 7.38 Hz), 5.96 (2H, s), 3.16 (3H, s), 2.29 (3H, s).

### N-(6-Methylpyridin-3-yl)-1-(4-(methylsulfonyl)benzyl)-1H-indazole-3-carboxamide (Compound 133)

The above compound was synthesised according to procedure B. LCMS RT=1.59 min, MH⁺=421.2, 99% UV purity. ¹H NMR (DMSO): 10.56 (1H, s), 8.90 (1H, d, J 2.49 Hz), 8.26 (1H, d, J 8.10 Hz), 8.16 (1H, dd, J 3.65 Hz), 7.87 (3H, m), 7.51 (3H, m), 7.35 (1H, t, J 7.51 Hz), 7.24 (1H, d, 8.49 Hz), 5.97 (2H, s), 3.00 (3H, s), 2.32 (3H, s).

### N-(Benzo[d][1,3]dioxol-5-yl)-1-(4-(methylsulfonyl)benzyl)-1H-pyrazole-3-carboxamide (Compound 148)

The title compound was prepared by subsequently following procedures B (with 1-(chloromethyl)-4-(methylsulfonyl)benzene and methyl 1H-pyrazole-3-carboxylate, 57% yield), C and P to afford 284 mg (78%, yield over last two steps). (LCMS RT=1.96 min, MH⁺=399.9), ¹H NMR (CDCl₃): 8.56 (1H, s), 7.97 (2H, d, J 8.4), 7.51 (1H, d, J 2.3), 7.45-7.37 (3H, br m), 7.00-6.94 (2H, br m), 6.79 (1H, d, J 8.2), 5.98 (2H, s), 5.47 (2H, s), 3.07 (3H, s).

### N-(Benzofuran-5-yl)-1-(4-(methylsulfonyl)benzyl)-1H-indazole-3-carboxamide (Compound 120)

The above compound was synthesised according to procedures A-D to afford 98 mg (73% final step). LCMS RT=2.43 min, MH⁺=446.1 ¹H NMR (DMSO): 10.42 (1H, s), 8.28 (1H, d, J 8.4 Hz), 8.25 (1H, d, J 2.1 Hz), 7.98 (1H, d, J 2.1 Hz), 7.90 (2H, d, J 8.3 Hz), 7.85 (1H, d, J 8.6 Hz), 7.72 (1H, dd, J 8.9, 2.1 Hz), 7.59-7.48 (4H, m), 7.35 (1H, t, J 7.6 Hz), 6.98 (1H, d, J 2.1 Hz), 5.97 (2H, s), 3.17 (3H, s).

### 1-(4-(Methylsulfonyl)benzyl)-N-(5,6,7,8-tetrahydronaphthalen-2-yl)-1H-indazole-3-carboxamide (Compound 121)

The above compound was synthesised according to procedures A-D to afford 93 mg (67% final step). LCMS RT=2.77 min, MH⁺=460.2 ¹H NMR (DMSO): 10.15 (1H, s), 8.25 (1H, d, J 8.2 Hz), 7.90 (2H, d, J 8.3 Hz), 7.84 (1H, d, J 8.5 Hz), 7.62 (1H, d, J 1.9 Hz), 7.55-7.50 (4H, m), 7.33 (1H, t, J 7.6 Hz), 7.01 (1H, d, J 8.3 Hz), 5.95 (2H, s), 3.16 (3H, s), 2.72-2.68 (4H, m), 1.74 (4H, m).

### N-(4-Cyclohexylphenyl)-1-(4-(methylsulfonyl)benzyl)-1H-indazole-3-carboxamide (Compound 122)

The above compound was synthesised according to procedures A-D to afford 82 mg (56% final step). LCMS RT=3.14 min, MH⁺=487.8 ¹H NMR (DMSO): 10.27 (1H, s), 8.26 (1H, d, J 8.1 Hz), 7.89 (2H, d, J 8.1 Hz), 7.83 (1H, d, J 8.2 Hz), 7.76 (2H, d, J 8.4 Hz), 7.52-7.47 (3H, m), 7.34 (1H, t, 7.9 Hz), 7.19 (2H, d, J 8.4 Hz), 5.96 (2H, s), 3.17 (3H, s), 1.80-1.17 (11H, m).

### N-(Benzo[d][1,3]dioxol-5-yl)-1-(4-(methylsulfonyl)benzyl)-4,5,6,7-tetrahydro-1H-indazole-3-carboxamide (Compound 149)

The above compound was synthesised according to procedures A-D to afford 18 mg (16% step A, 24% step B, 92% step C, 36% step D). LCMS RT=2.29 min, MH⁺=454.3 ¹H NMR (DMSO): 9.92 (1H, s), 7.87 (2H, d, J 8.3 Hz), 7.44 (2H, d, J 8.2 Hz), 7.31 (1H, s), 7.06 (1H, d, J 8.5 Hz), 6.88 (1H, d, J 8.4 Hz), 6.00 (2H, s), 5.55 (2H, s), 3.18 (3H, s), 2.68-2.58 (4H, m), 1.71 (4H, m).

### N-(Benzo[d][1,3]dioxol-5-yl)-1-(4-(methylsulfonyl)benzyl)-1,4,5,6-tetrahydrocyclopenta[c]pyrazole-3-carboxamide (Compound 150)

The above compound was synthesised according to procedures A-D to afford 43 mg (20% step A, 48% step B, 100% step C, 34% step D). LCMS RT=2.20 min, MH⁺=440.3 ¹H NMR (DMSO): 9.86 (1H, s), 7.95 (2H, d, J 8.2 Hz), 7.52 (2H, d, J 8.2 Hz), 7.37 (1H, d, J 1.7 Hz), 7.14 (1H, dd, J 8.4, 1.8 Hz), 6.96 (1H, d, J 8.4 Hz), 6.08 (2H, s), 5.71 (2H, s), 3.26 (3H, s), 2.92 (2H, t, J 7.1 Hz), 2.76 (2H, t, J 7.2 Hz), 2.50-2.43 (2H, m).

### N-(2,3-Dihydro-1H-inden-5-yl)-1-(4-(methylsulfonyl)benzyl)-1H-indazole-3-carboxamide (Compound 130)

The above compound was synthesised according to procedures A-D to afford 95 mg (71% final step). LCMS RT=2.65 min, MH⁺=446.2 ¹H NMR (DMSO): 10.21 (1H, s), 8.25 (1H, d, J 8.1 Hz), 7.89 (2H, d, J 8.4 Hz), 7.84 (1H, d, J 8.6 Hz), 7.79 (2H, s), 7.59-7.47 (4H, m), 7.34 (1H, t, J 7.4 Hz), 7.18 (1H, d, J 8.2 Hz), 5.96 (2H, s), 3.17 (3H, s), 2.89-2.81 (4H, m), 2.05-2.00 (2H, m).

### N-(Benzo[d][1,3]dioxol-5-yl)-1-(4-methylbenzyl)-1H-indazole-3-carboxamide (Compound 131)

The above compound was synthesised according to procedures A-D to afford 85 mg (84% step B, 76% step C, 30% step D). LCMS RT=2.79 min, MH⁺=386.1 ¹H NMR (DMSO): 10.27 (1H, s), 8.22 (1H, d, J 8.2 Hz), 7.77 (1H, d, J 8.5 Hz), 7.54 (1H, d, J 2.0 Hz), 7.46 (1H, t, J 7.1 Hz), 7.35 (1H, dd, J 8.5, 2.1 Hz), 7.30 (1H, t, J 7.6 Hz), 7.20-7.11 (4H, m), 6.90 (1H, d, J 8.4 Hz), 6.01 (2H, s), 5.76 (2H, s), 2.24 (3H, s).

### N-(Benzo[d][1,3]dioxol-5-yl)-1-(4-ethylbenzyl)-1H-indazole-3-carboxamide (Compound 132)

The above compound was synthesised according to procedures A-D to afford 33 mg (75% step B, 100% step C, 10% step D). LCMS RT=2.95 min, MH⁺=400.1 ¹H NMR (DMSO): 10.27 (1H, s), 8.22 (1H, d, J 8.1 Hz), 7.79 (1H, d, J 8.6 Hz), 7.54 (1H, d, J 2.0 Hz), 7.47 (1H, t, J 7.2 Hz), 7.37-7.28 (2H, m), 7.23-7.15 (4H, m), 6.90 (1H, d, J 8.4 Hz), 6.01 (2H, s), 5.77 (2H, s), 2.54 (2H, q, J 7.7 Hz), 1.11 (3H, t, 7.6 Hz).

### Example 5

### Preparation of 1-(4-(Methylsulfonyl)benzyl)-N-(naphthalen-2-yl)-1H-indazole-3-carboxamide (Compound 116)

### Tert-butyl naphthalen-2-ylcarbamate

Procedure E: A solution of 2-naphthoic acid (100 mg, 0.58 mmol) in t-butanol (4 ml) was stirred in the presence of 4A Molecular Sieves (crushed & activated) for 30 minutes. Diphenylphosphoryl azide (0.124 mL, 0.58 mmol) and triethylamine (0.081 mL, 0.58 mmol) were then added and the resulting mixture was heated at 80° C. for 16 h. After concentration of the reaction mixture in vacuo the residue was purified by column chromatography eluting using a gradient (petroleum ether 40/60/ethyl acetate 1:0 v/v 1:1 v/v) which afforded 129 mg (91%) of the title compound.

¹H NMR (CDCl₃): 7.91 (1H, s), 7.69-7.63 (3H, br m), 7.37-7.12 (3H, br m), 6.62 (1H, s), 3.90-3.15, 8H, m). 1.47 (9H, s).

### 1-(4-(Methylsulfonyl)benzyl)-N-(naphthalen-2-yl)-1H-indazole-3-carboxamide (Compound 116)

Procedure F: Tert-butyl naphthalen-2-ylcarbamate (128 mg, 0.53 mmol) was stirred in a 1:1 mixture of dichloromethane and trifluoroacetic acid (1 ml each) for 30 minutes at room temperature. The reaction mixture was then diluted with toluene, concentrated and traces of trifluoroacetic acid were removed by co-evaporating with toluene (twice). To the residue 1-(4-(methylsulfonyl)benzyl)-1H-indazole-3-carboxylic acid (132 mg, 0.40 mmol), (benzotriazol-1-yloxy)tripyrrolidinophosphonium hexafluorophosphate (250 mg, 0.48 mmol), diisopropylethylamine (0.21 mL, 1.20 mmol) and N,N-dimethylformamide (5 mL) were added and the resulting mixture was stirred at room temperature for 16 h. The reaction mixture was partitioned between ethyl acetate and water, the organic phase washed with water (2×), the aqueous phase extracted with ethyl acetate (1×) and the combined organic phases washed with brine, dried over magnesium sulfate and concentrated. Purification by column chromatography eluting using a gradient (petroleum ether 40/60/ethyl acetate 1:0 v/v to 3:7 v/v) afforded 149 mg (77%) of the title compound. (LCMS RT=2.61 min, MH⁺=456.1), ¹H NMR (CDCl₃): 9.02 (1H, s), 8.57-8.49 (2H, br m), 7.94 (2H, d, J 8.4), 7.91-7.80 (3H, br m), 7.70 (1H, dd, J 8.9 and 2.1), 7.56-7.36 (7H, br m), 5.78 (2H, s), 3.05 (3H, s).

### Example 6

### Preparation of N-((1-(4-(Methylsulfonyl)benzyl)-1H-indazol-3-yl)methyl)benzo[d][1,3]-dioxol-5-amine (Compound 90)

Procedure G: A solution of N-(benzo[d][1,3]dioxol-5-yl)-1-(4-(methylsulfonyl)benzyl)-1H-indazole-3-carboxamide (87 mg, 0.19 mmol) in tetrahydrofuran (1 ml) was cooled to 0° C. under argon atmosphere. Borane tetrahydrofuran complex (0.97 ml of 1 M solution, 0.97 mmol) was added and the resulting mixture was heated to reflux for 16 h. After cooling to room temperature sodium hydroxide (aq, 1M, 2 ml) was added and the reaction mixture heated to reflux for 2 h. After cooling to room temperature the reaction mixture was partitioned between ethyl acetate and water. Aqueous layer was extracted with ethyl acetate and the combined organic layers were washed with brine, dried (magnesium sulfate) and concentrated in vacuo. Purification by preparative HPLC afforded 52 mg (63%) of the title compound. (LCMS RT=5.71 min, MH⁺=436.2), ¹H NMR (CDCl₃): 7.77 (2H, d, J 8.3), 7.65 (1H, d, J 8.2), 7.36-7.29 (1H, br m), 7.26-7.16 (3H, br m), 7.12 (1H, t, J 7.4), 6.63-6.58 (1H, br m), 6.55 (1H, br s), 6.50-6.43 (1H, br m), 5.85 (2H, s), 5.56 (2H, s), 4.68 (2H, s), 2.95 (3H, s).

### Example 7

### Preparation of 6-Chloro-2-(1-(4-(methylsulfonyl)benzyl)-1H-indazol-3-yl)benzo[d]oxazole (Compound 95)

Procedure H: A mixture of 1-(4-(methylsulfonyl)benzyl)-1H-indazole-3-carboxylic acid (200 mg, 0.605 mmol), 2-amino-5-chlorophenol (87 mg, 0.605 mg) and polyphosphoric acid (5 ml) was heated at 120° C. for 3 h. The reaction mixture was then poured into ice water, neutralized with potassium carbonate and filtered. Purification by column chromatography eluting using a gradient (petroleum ether 40/60/ethyl acetate 1:0 v/v to 1:9 v/v) followed by two recrystallisations with ethyl acetate afforded 32 mg (12%) of the title compound. (LCMS RT=7.14 min, MH⁺=438.2), ¹H NMR (DMSO-d6): 8.41 (1H, d, J 8.1), 8.07 (1H, d, J 1.9), 7.98 (1H, d, J 8.5), 7.94-7.87 (3H, br m), 7.64-7.55 (3H, br m), 7.54-7.42 (2H, br m), 6.01 (2H, s), 3.17 (3H, s).

### Example 8

### Preparation of 1-(4-(Methylsulfonyl)benzyl)-N-(4-(methylsulfonyl)phenyl)-1H-indazole-3-carboxamide (Compound 101)

Procedure J: To a stirred mixture of 1-(4-(methylsulfonyl)benzyl)-1H-indazole-3-carboxylic acid (100 mg, 0.30 mmol) and oxalylchloride (0.045 mL, 0.36 mmol) in dichloromethane (2 mL) was added a drop of N,N-dimethylformamide and the resulting mixture was stirred at room temperature for 1 h. 4-(methylsulfonyl)aniline (62 mg, 0.36 mmol) and diisopropylethylamine (0.104 mL, 0.60 mmol) were then added and the reaction mixture was stirred for 16 h at room temperature. Concentration in vacuo and purification by column chromatography eluting using a gradient (petroleum ether 40/60/ethyl acetate 1:0 v/v to 2:8 v/v) afforded 100 mg (69%) of the title compound. (LCMS RT=5.24 min, MH⁺=483.7), ¹H NMR (CDCl₃): 9.08 (1H, br s), 8.51-8.45 (1H, br m), 7.99 (4H, s), 7.94 (2H, d, J 8.5), 7.54-7.47 (1H, br m), 7.45-7.36 (4H, br m), 5.78 (2H, s), 3.09 (3H, s), 3.05 (3H, s).

### Example 9

### Preparation of N-(Benzo[d][1,3]dioxol-5-yl)-1-(4-(methylsulfonyl)benzyl)-1H-indazole-3-carbothioamide (Compound 102)

Procedure K: A mixture of N-(benzo[d][1,3]dioxol-5-yl)-1-(4-(methylsulfonyl)benzyl)-1H-indazole-3-carboxamide (130 mg, 0.29 mmol) and Lawesson's reagent (70 mg, 0.174 mmol) were heated in a mixture toluene (2.5 mL) and 1,4-dioxane (0.5 mL) at 110° C. for 16 h. Concentration in vacuo, purification by column chromatography eluting using a gradient (petroleum ether 40/60/ethyl acetate 1:0 v/v to 3:7 v/v) followed by further purification by preparative HPLC afforded 17 mg (13%) of the title compound. (LCMS RT=6.31 min, MH⁺=466.3), ¹H NMR (CDCl₃): 10.27 (1H, br s), 8.90 (1H, d, J 8.1), 7.83 (2H, d, J 8.4), 7.50 (1H, d, J 2.1), 7.42-7.24 (4H, br m—obscured by NMR solvent signal), 7.19 (1H, s), 7.08 (1H, dd, J 8.5 and 2.2), 6.79 (1H, d, J 8.3), 5.95 (2H, s), 5.65 (2H, s), 2.95 (3H, s).

### Example 10

### Preparation of N-(1-(4-(Methylsulfonyl)benzyl)-1H-indazol-3-yl)benzo[d][1,3]-dioxole-5-carboxamide (Compound 114)

### Tert-butyl 1-(4-(methylsulfonyl)benzyl)-1H-indazol-3-ylcarbamate

The title compound was prepared according to procedure E. Concentration of the reaction mixture in vacuo the residue was purified by column chromatography eluting using a gradient (40/60 petroleum ether/ethyl acetate 1:0 v/v 3:7 v/v) afforded 94 mg (78%) of the corresponding Boc protected amine. ¹H NMR (CDCl₃): 7.99 (1H, d, J 8.4), 7.82 (2H, d, J 8.5), 7.62 (1H, s), 7.39-7.27 (3H, br m), 7.22 (1H, d, J 8.5), 7.14 (1H, td, J 7.5 and 0.9), 5.54 (2H, s), 2.99 (3H, s), 1.54 (9H, s).

### N-(1-(4-(Methylsulfonyl)benzyl)-1H-indazol-3-yl)benzo[d][1,3]dioxole-5-carboxamide (Compound 114)

The title compound was prepared according to procedure F. Purification by column chromatography eluting using a gradient (petroleum ether/ethyl acetate 1:0 v/v to 3:7 v/v) followed by trituration petroleum ether/ethyl acetate afforded 56 mg (54%) of the title compound. (LCMS RT=2.10 min, MH⁺=450.2), ¹H NMR (CDCl₃): 8.40 (1H, br s), 8.16 (1H, d, J 8.3), 7.90 (2H, d, J 8.4), 7.53 (1H, dd, J 8.2 and 1.9), 7.49-7.37 (4H, br m), 7.33-7.29 (1H, br m—overlapping with NMR solvent signal on right hand side), 7.22 (1H, t, J 7.5), 6.92 (1H, d, J 8.1), 6.10 (2H, s), 5.60 (2H, s), 3.03 (3H, s).

### Example 11

### Preparation of 1-(4-chlorophenyl)-3-(1-(4-(methylsulfonyl)benzyl)-1H-indazol-3-yl)urea (Compound 118)

Procedure L: Tert-butyl 1-(4-(methylsulfonyl)benzyl)-1H-indazol-3-ylcarbamate (0.30 mmol) was stirred in a 1:1 mixture of dichloromethane and trifluoroacetic acid (1 ml each) at room temperature. The reaction mixture was then diluted with toluene, concentrated and traces of trifluoroacetic acid were removed by co-evaporating with toluene (twice). To the residue redissolved in dichloromethane (2 ml), 1-chloro-4-isocyanatobenzene (55 mg, 0.36 mmol) and triethylamine (0.42 mL, 0.30 mmol) were added and the resulting mixture was stirred at room temperature for 16 h. The solids were collected by filtration and repetitive recrystalisations with ethyl acetate afforded 52 mg (38%) of the title compound. (LCMS RT=2.23 min, MH⁺=455.1), ¹H NMR (DMSO-d6): 9.85 (1H, br. s), 9.75 (1H, br s), 8.14-7.01 (12H, br m), 5.71 (2H, s), 3.16 (3H, s—obscured by water signal on left hand side).

### Example 12

### Preparation of N-(4-Isopropylphenyl)-1-(4-(morpholine-4-carbonyl)benzyl)-1H-indazole-3-carboxamide (Compound 19)

### (4-(Chloromethyl)phenyl)(morpholino)methanone

Procedure M: To a stirred mixture of 4-bromomethylbenzoic acid (0.5 mL, 2.3 mmol) and oxalylchloride (0.344 mL, 2.76 mmol) in dichloromethane (5 mL) was added a drop of N,N-dimethylformamide and the resulting mixture was stirred at room temperature for 1 h. Morpholine (0.46 mL, 4.6 mmol) and diisopropylethylamine (0.80 mL, 4.6 mmol) were then added and the reaction mixture was stirred for 16 h at room temperature. After concentration in vacuo and purification by column chromatography eluting using a gradient (40/60 petroleum ether/ethyl acetate 1:0 v/v to 0:1 v/v) afforded 467 mg (85%) of the title compound. (LCMS RT=12.09 min, MH⁺=240.2), ¹H NMR (CDCl₃): 7.39-7.30 (4H, m), 4.52 (2H,$), 3.90-3.15, 8H, m).

### Methyl 1-(4-(morpholine-4-carbonyl)benzyl)-1H-indazole-3-carboxylate

Following procedure B 72 mg (42%) the title compound was obtained (purification carried out by column chromatography eluting using a gradient (40/60 petroleum, ether/ethyl acetate 1:0 v/v to 2:8 v/v). (LCMS RT=7.15 min, 2M+H⁺=759.2), ¹H NMR (CDCl₃): 8.23 (1H, d, J 8.0), 7.36-7.22 (4H, m), 5.71 (2H,$), 4.04 (3H, s), 3.67-3.38 (8H, m).

### 1-(4-(Morpholine-4-carbonyl)benzyl)-1H-indazole-3-carboxylic acid

Following procedure C (starting from methyl 1-(4-(morpholine-4-carbonyl)benzyl)-1H-indazole-3-carboxylate) the title compound was isolated with quantitative yield and used in the next step without further purification.

### N-(4-Isopropylphenyl)-1-(4-(morpholine-4-carbonyl)benzyl)-1H-indazole-3-carboxamide (Compound 19)

Procedure N: 1-(4-(morpholine-4-carbonyl)benzyl)-1H-indazole-3-carboxylic acid

(0.19 mmol) was taken up in N,N-dimethylformamide (1.5 mL), 4-isopropylaniline (31 mL, 0.23 mmol), 2-(1H-7-Azabenzotriazol-1-yl)-1,1,3,3-tetramethyl uronium hexafluorophosphate methanaminium (87 mg, 0.23 mmol) and diisopropylethylamine (0.1 mL, 0.57 mmol) were added and the resulting reaction mixture was stirred for 16 h at room temperature. The reaction mixture was partitioned between ethyl acetate and water, the organic phase washed with water (2×), the aqueous phase extracted with ethyl acetate (1×) and the combined organic phases washed with brine, dried over magnesium sulfate and concentrated. The residue was purified by column chromatography eluting using a gradient (petroleum ether 40/60/ethyl acetate 1:0 v/v to 0:1 v/v) to afford 50 mg (55%) of the title compound. (LCMS RT=6.92 min, MH⁺=483.2), ¹H NMR (CDCl₃): 8.83 (1H, s), 8.47 (1H, d, J 8.1), 7.38 (2H, d, J 8.5), 7.46-7.24 (9H, m), 5.67 (2H, s), 4.04 (3H, s), 3.74-3.42 (8H, m), 2.92 (1H, m), 1.29 (3H, s), 1.26 (3H, s).

### Example 13

### Preparation of N-(2,2-Difluorobenzo[d][1,3]-dioxol-5-yl)-1-(4-(morpholine-4-carbonyl)benzyl)-1H-indazole-3-carboxamide (Compound 25)

Procedure P: 1-(4-(morpholine-4-carbonyl)benzyl)-1H-indazole-3-carboxylic acid

(0.30 mmol) was taken up in N,N-dimethylformamide (2 mL), 2,2-difluorobenzo[d][1,3]dioxol-5-amine (62 mg, 0.36 mmol), (benzotriazol-1-yloxy)tripyrrolidinophosphonium hexafluorophosphate (187 mg, 0.36 mmol) and diisopropylethylamine (0.157 mL, 0.90 mmol) were added and the resulting mixture was stirred at room temperature for 16 h. The reaction mixture was partitioned between ethyl acetate and water, the organic phase washed with water (2×), the aqueous phase extracted with ethyl acetate (1×) and the combined organic phases washed with brine, dried over magnesium sulfate and concentrated. Purification by column chromatography eluting using a gradient (40/60 petroleum ether/1:0 v/v to 0:1 v/v) followed by preparative HPLC afforded 20 mg (13%). (LCMS RT=6.62 min, MH⁺=520.8), ¹H NMR (CDCl₃): 8.89 (1H, s), 8.44 (1H, d, J 8.1), 7.87 (1H, d, J 2.0), 7.48-7.31 (5H, br m), 7.29-7.19 (3H, br m), 7.03 (1H, d, J 8.7), 5.67 (2H, s), 3.91-3.25 (8H, br m).

The following compounds were prepared according to procedure P as the last step. Purification of the final products to required purity specifications has been carried out by column chromatography and/or trituation (s)/recrystalisation(s).

### N-(Benzo[d][1,3]dioxol-5-yl)-1-(4-(morpholine-4-carbonyl)phenethyl)-1H-indazole-3-carboxamide (Compound 92)

The title compound was prepared by subsequently following procedures M (92% yield), B (yield 30%), C and P to afford 116 mg (78%). (LCMS RT=9.95 min, MH⁺=not found), ¹H NMR (CDCl₃): 8.74 (1H, s), 8.40 (1H, d, J 7.8), 7.52 (1H, d, J 2.1), 7.35-7.16 (5H, br m, partially obscured by NMR solvent signal) 7.15-7.04 (3H, br m), 6.84 (1H, d, J 8.3), 6.01 (2H, s), 4.67 (2H, t, J 7.1), 3.89-3.19 (10H, br m).

### N-(Benzo[d][1,3]dioxol-5-yl)-1-(4-(piperidine-1-carbonyl)benzyl)-1H-indazole-3-carboxamide (Compound 93)

The title compound was prepared by subsequently following procedures M (from piperidine with 64% yield), B (78% yield), C and P to afford 161 mg (39% over last 2 steps). LCMS: RT 6.53 min, MH⁺483.3 ¹H NMR (DMSO-d6): 10.30 (1H, s), 8.25 (1H, d, J 8.1 Hz), 7.84 (1H, d, J 8.6 Hz), 7.54 (1H, d, J 2.0 Hz), 7.52-7.46 (1H, m), 7.37-7.29 (6H, m), 6.90 (1H, d, J 8.4 Hz), 6.01 (2H, s), 5.86 (2H, s), 3.53 (2H, s), 3.20 (2H, s), 1.57-1.41 (6H, m).

### N-(Benzo[d][1,3]dioxol-5-yl)-1-(3-(morpholine-4-carbonyl)benzyl)-1H-indazole-3-carboxamide (Compound 100)

The title compound was prepared by subsequently following procedures M (from 3-(chloromethyl)benzoic acid with 72% yield), B (54% yield), C and P to afford 181 mg (61% over last 2 steps). (LCMS RT=5.91 min, MH⁺=485), ¹H NMR (CDCl₃): 8.76 (1H, br s), 8.47 (1H, d, J 8.1), 7.51 (1H, d, J 2.1), 7.48-7.21 (7H, br s—overlapping with NMR solvent signal), 7.06 (1H, dd, J 8.5 and 2.1), 6.83 (1H, d, J 8.3), 6.00 (2H, s), 5.69 (2H, s), 3.91-3.18 (8H, br m).

### N-(Benzo[d][1,3]dioxol-5-yl)-1-(4-(dimethylcarbamoyl)benzyl)-1H-indazole-3-carboxamide (Compound 117)

The title compound was prepared by subsequently following procedures M (from dimethylamine with 41% yield), B (30% yield), C and P to afford 85 mg (25% over last 2 steps). LCMS: RT 2.10 min, MH⁺443.3 ¹H NMR (DMSO-d6): 10.12 (1H, s), 8.07 (1H, d, J 8.2), 7.65 (1H, d, J 8.5), 7.37 (1H, d, J 2.1), 7.31 (1H, t, J 7.6), 7.21-7.10 (6H, br m), 6.72 (1H, d, J 8.5), 5.83 (2H, s), 5.69 (2H, s), 2.80-2.76 (3H, s), 2.66 (3H, s).

### N-(Benzo[d][1,3]dioxol-5-yl)-1-benzyl-1H-indazole-3-carboxamide (Compound 87)

The title compound was prepared by subsequently following procedures B (with benzylbromide), C and P to afford 112 mg (75%, yield over last two steps). (LCMS RT=6.97 min, MH⁺=371.9), ¹H NMR (CDCl₃): 8.79 (1H, s), 8.46 (1H, d, J 8.1), 7.51 (1H, d, J 2.1), 7.46-7.30 (6H, br m), 7.25-7.19 (2H, br s), 7.06 (1H, dd, J 8.3 and 2.1), 6.82 (1H, d, J 8.4), 6.00 (2H, s), 5.67 (2H, s).

### N-(Benzo[d][1,3]dioxol-5-yl)-1-(pyridin-4-ylmethyl)-1H-indazole-3-carboxamide (Compound 97)

The title compound was prepared by subsequently following procedures B (with 4-(bromomethyl)pyridine hydrobromide), C and P to afford 53 mg (36%, yield over last two steps). (LCMS RT=4.80 min, MH⁺=373.1), ¹H NMR (CDCl₃): 8.63 (1H, br m), 8.40 (1H, d, J 8.1), 7.42-7.34 (2H, br m), 7.31-7.22 (2H, br m), 7.18 (2H, s), 7.07-6.92 (3H, br m), 6.72 (1H, d, J 8.4), 5.90 (2H, s), 5.59 (2H, s).

### N-(Benzo[d][1,3]dioxol-5-yl)-1-(pyridin-3-ylmethyl)-1H-indazole-3-carboxamide (Compound 106)

The title compound was prepared by subsequently following procedures B (with 3-(chloromethyl)pyridine hydrochloride), C and P to afford 218 mg (71%, yield over last two steps). (LCMS RT=1.88 min, MH⁺=373), ¹H NMR (CDCl₃): 8.74 (1H, s), 8.66 (1H, s), 8.59 (1H, d, J 4.8), 8.47 (1H, d, J 8.2), 7.54-7.41 (6H, br m—overlapping with NMR solvent signal), 7.05 (1H, dd, J 8.3 and 2.2), 6.82 (1H, d, J 8.3), 6.00 (2H, s), 5.68 (2H, s).

### N-(Benzo[d][1,3]dioxol-5-yl)-1-(4-(trifluoromethyl)benzyl)-1H-indazole-3-carboxamide (Compound 115)

The title compound was prepared by subsequently following procedures B (with 1-(chloromethyl)-4-(trifluoromethyl)benzene), C and P to afford 138 mg (57%, yield over last two steps). (LCMS RT=2.79 min, MH⁺=440.2), ¹H NMR (CDCl₃): 8.75 (1H, br s), 8.51-8.46 (1H, br m), 7.61 (2H, d, J 8.2), 7.51 (1H, d, J 3.1), 7.49-7.42 (1H, br m), 7.39-7.28 (4H, br m—overlapping with NMR solvent signal at left hand side), 7.05 (1H, dd, J 8.4 and 2.2), 6.82 (1H, d, J 8.3), 6.00 (2H, s), 5.73 (2H, s).

### N-(4-Acetamidophenyl)-1-(4-(trifluoromethyl)benzyl)-1H-indazole-3-carboxamide (Compound 109)

The title compound was prepared by subsequently following procedures B (with 1-(chloromethyl)-4-(trifluoromethyl)benzene), C and P to afford 72 mg (51%, yield over last two steps). (LCMS RT=2.41 min, MH⁺=453.1), ¹H NMR (CD₃OD): 8.22 (1H, d, J 8.1), 7.62 (2H, d, J 8.8), 7.56-7.43 (5H, br m), 7.40-7.30 (3H, br m), 7.23 (1H, t, J 7.4), 5.78 (2H, s), 2.03 (3H, s).

### 1-(4-Isopropylbenzyl)-N-(4-(methylsulfonyl)phenyl)-1H-indazole-3-carboxamide (Compound 91)

The title compound was prepared by subsequently following procedures B (with 1-(chloromethyl)-4-isopropylbenzene), C and P to afford 30 mg (19%, yield over last two steps). (LCMS RT=10.85 min, MH⁺=not found), ¹H NMR (CDCl₃): 9.14 (1H, s), 8.44 (1H, d, J 8.1), 8.03-7.95 (4H, br m), 7.46 (2H, d, J 3.9), 7.41-7.33 (1H, br m), 7.26-7.14 (4H, br m), 5.66 (2H, s), 3.09 (3H, s), 2.98-2.82 (1H, br m), 1.23 (6H, d, J 6.9).

### N-(Benzo[d][1,3]dioxol-5-yl)-1-(4-isopropylbenzyl)-1H-indazole-3-carboxamide (Compound 119)

The title compound was prepared by subsequently following procedures B (with 1-(chloromethyl)-4-isopropylbenzene with 71% yield), C and P to afford 193 mg (58%, yield over last two steps). (LCMS RT=3.06 min, MH⁺=414.2), ¹H NMR (DMSO-d6): 10.29 (1H, s), 8.22 (1H, d, J 8.2), 7.82 (1H, d, J 8.5), 7.55 (1H, d, J 2.1), 7.47 (1H, t, J 7.7), 7.38-7.27 (2H, br m), 7.25-7.17 (4H, br m), 6.90 (1H, d, J 8.5), 6.01 (2H, s), 5.77 (2H, s), 2.88-2.77 (1H, m), 1.14 (6H, d, J 6.9).

### N-(Benzo[d][1,3]dioxol-5-yl)-1-(4-tert-butylbenzyl)-1H-indazole-3-carboxamide (Compound 42)

The title compound was prepared by subsequently following procedures B (with 1-(bromomethyl)-4-tert-butylbenzene with 68% yield), C and P to afford 182 mg (55%, yield over last two steps). (LCMS RT=3.19 min, MH⁺=428.4), ¹H NMR (DMSO-d6): 10.27 (1H, s), 8.23 (1H, d, J 8.1), 7.82 (1H, d, J 8.5), 7.55 (1H, d, J 2.1), 7.48 (1H, t, J 7.7), 7.37-7.28 (4H, br m), 7.22 (2H, d, J 8.4), 6.90 (1H, d, J 8.5), 6.01 (2H, s), 5.77 (2H, s), 1.22 (9H, s).

### N-(Benzo[d][1,3]dioxol-5-yl)-1-(2-(methylsulfonyl)benzyl)-1H-indazole-3-carboxamide (Compound 136)

The title compound was prepared from 1-(chloromethyl)-2-(methylsulfonyl)benzene by subsequently following procedures B (yield 77%), C and P to afford 90 mg (64%). LCMS RT=2.45 min, MH⁺=450.0 ¹H NMR (DMSO-d6): 10.28 (1H, br s), 8.29 (1H, d, J 8.3), 8.08-8.01 (1H, br m), 7.78 (1H, d, J 8.6), 7.61-7.48 (4H, br m), 7.40-7.30 (2H, br m), 6.89 (1H, d, J 8.4), 6.57-6.51 (1H, br m), 6.33 (2H, s), 6.01 (2H, s), 3.49 (3H, s).

### N-(Benzo[d][1,3]dioxol-5-yl)-1-(4-(morpholinosulfonyl)benzyl)-1H-indazole-3-carboxamide (Compound 137)

The title compound was prepared from 4-{[4-(bromomethyl)phenyl]sulfonyl}morpholine by subsequently following procedures B (yield 48%), C and P to afford 90 mg (67%). LCMS RT=1.20 min, MH⁺=521.0 ¹H NMR (DMSO-d6): 10.31 (1H, br s), 8.26 (1H, d, J 8.0), 7.83 (1H, d, J 8.6), 7.72 (2H, d, J 8.3), 7.55-7.45 (4H, br m), 7.38-7.31 (2H, br m), 6.90 (1H, d, J 8.4), 6.01 (2H, s), 5.98 (2H, s), 3.62-3.54 (4H, br m), 2.84-2.78 (4H, br m).

### Example 13

### Preparation of N-(Benzo[d][1,3]-dioxol-5-yl)-1-(thiophen-3-ylmethyl)-1H-indazole-3-carboxamide (Compound 129)

Procedure R: To a stirred solution of thiophen-3-ylmethanol (2 mmol, 0.29 g) in dry DCM (20 mL) under an inert atmosphere was added phosphorous tribromide (4 mmol, 0.38 mL). The reaction mixture was stirred at room temperature for 16 h, and then concentrated in vacuo and partitioned between saturated sodium bicarbonate solution and DCM. The layers were separated and the aqueous was extracted twice more with DCM. The combined organic layers were washed with brine, dried over MgSO₄ and concentrated in vacuo to give a quantitative yield of 3-(bromomethyl)thiophene. ¹H NMR (CDCl₃): 7.09 (2H, m), 6.94 (1H, dd, J 4.9, 1.3 Hz), 4.33 (2H, s).

Subsequent steps were performed according to the general procedure to afford 61 mg (69% step B, 100% step C, 69% crude yield dropping to 14% after purification in step D). LCMS RT=2.61 min, MH⁺=378.1 ¹H NMR (DMSO): 10.27 (1H, s), 8.22 (1H, d, J 8.2 Hz), 7.84 (1H, d, J 8.6 Hz), 7.55 (1H, d, J 2.1 Hz), 7.51-7.45 (3H, m), 7.37-7.28 (2H, m), 7.07 (1H, dd, J 4.6, 1.6 Hz), 6.90 (1H, d, J 8.4 Hz), 6.01 (2H, s), 5.79 (2H, s).

The following compounds were prepared by procedures set out in the earlier examples.

### N-(Benzo[d][1,3]dioxol-5-yl)-1-(4-(pyrrolidine-1-carbonyl)benzyl)-1H-indazole-3-carboxamide (Compound 142)

The above compound was synthesised according to procedures M (with pyrrolidine), B, C then D to afford 46 mg (29% step M, 44% step B, 64% step C and 52% step D). LCMS RT=2.35 min, MH⁺=469.4 ¹H NMR (DMSO): 10.31 (1H, s), 8.25 (1H, d, J 8.1 Hz), 7.83 (1H, d, J 8.5 Hz), 7.55-7.46 (4H, m), 7.37-7.28 (4H, m), 6.90 (1H, d, J 8.5 Hz), 6.00 (2H, s), 5.87 (2H, s), 3.42 (2H, t, J 6.9 Hz), 3.31 (2H, t, J 7.3 Hz), 1.79 (4H, m).

### N-(2,3-Dihydrobenzo[b][1,4]dioxin-6-yl)-1-(4-(morpholine-4-carbonyl)benzyl)-1H-indazole-3-carboxamide (Compound 143)

The above compound was synthesised according to procedures M, B, C then D to afford 75 mg (91% step M, 31% step B, 98% step C, 56% step D). LCMS RT=2.24 min, MH⁺=499.4 ¹H NMR (DMSO): 10.23 (1H, s), 8.24 (1H, d, J 8.2 Hz), 7.83 (1H, d, J 8.6 Hz), 7.51-7.46 (2H, m), 7.39-7.30 (6H, m), 6.82 (1H, d, J 8.8 Hz), 5.86 (2H, s), 4.27-4.19 (4H, m), 3.63-3.32 (8H, m).

### N-(4-Methoxyphenyl)-1-(4-(morpholine-4-carbonyl)benzyl)-1H-indazole-3-carboxamide (Compound 144)

The above compound was synthesised according to procedures M, B, C then D to afford 84 mg (66% yield final step). LCMS RT=2.27, MH⁺=471.4 ¹H NMR (DMSO): 10.25 (1H, s), 8.25 (1H, d, J 8.0 Hz), 7.84 (1H, d, J 8.6 Hz), 7.78 (2H, d, J 9.0 Hz), 7.49 (1H, t, J 8.2 Hz), 7.40-7.30 (5H, m), 6.93 (2H, d, J 9.1 Hz), 5.86 (2H, s), 3.75 (3H, s), 3.63-3.31 (8H, m).

### 1-(4-(Morpholine-4-carbonyl)benzyl)-N-(4-(trifluoromethoxy)phenyl)-1H-indazole-3-carboxamide (Compound 61)

The above compound was synthesised according to procedures M, B, C then D to afford 73 mg (52% yield final step). LCMS RT=tbd, MH⁺=tbd ¹H NMR (DMSO): 10.63 (1H, s), 8.26 (1H, d, J 8.1 Hz), 8.02 (2H, d, J 9.1 Hz), 7.85 (1H, d, J 8.5 Hz), 7.53-7.32 (8H, m), 5.89 (2H, s), 3.56-3.29 (8H, m).

### Example 14

### Preparation of N-(benzo[d][1,3]-dioxol-5-yl)-1-(3-(methylsulfonyl)benzyl)-1H-indazole-3-carboxamide (Compound 127)

### 1-(Chloromethyl)-3-(methylsulfonyl)benzene

Borane solution (1M in THF) was added dropwise to a stirred mixture of 3-methylsulfonyl)benzoic acid (400 mg, 2.00 mmol) in anhydrous THF (2 ml) at 0° C. The resulting mixture was then allowed to warm to room temperature and stirred for 3 h. The reaction mixture quenched with water/1 M HCl (aq.), extracted with ethyl acetate (3 times), the combined organic extracts dried (sodium sulfate) and concentrated in vacuo to give crude (3-(methylsulphonyl)phenyl)methanol as an oil which was used in the next step without further purification. The alcohol was dissolved in dichloromethane (6 ml) and triethylamine (0.418 mL, 3.0 mmol). Methanesulfonylchloride (first batch (0.186 mL, 2.4 mmol) was then added, followed by a second batch (0.039 mL, 0.5 mmol) and the resulting mixture was stirred for 16 h. 1 N HCl solution (aq.) was then added and the reaction mixture was extracted with diethyl ether (3 times), the combined organic extracts were dried (sodium sulfate) and concentrated in vacuo. The residue was purified by column chromatography eluting using a gradient (petroleum ether 40/60/ethyl acetate 1:0 v/v 0:1) to afford 212 mg (52% over 2 steps) of the title compound as an oil. ¹H NMR (CDCl₃): 7.98 (1H, s), 7.91 (1H, d, J 7.8), 7.70 (1H, d, J 7.8), 7.59 (1H, t, J 7.8), 4.65 (2H, s), 3.08 (s, 3H).

### N-(Benzo[d][1,3]dioxol-5-yl)-1-(3-(methylsulfonyl)benzyl)-1H-indazole-3-carboxamide (Compound 127)

The title compound was prepared from 1-(chloromethyl)-3-(methylsulfonyl)benzene by subsequently following procedures B (yield 42%), C and P afford 90 mg (46%). (LCMS RT=2.34 min, MH⁺=450.1), ¹H NMR (CDCl₃): 8.74 (1H, s), 8.49 (1H, d, J 8.2), 7.97 (1H, s), 7.91 (1H, d, J 8.0), 7.59-7.33 (6H, br m), 7.06 (1H, d, J 8.0 and 2.1), 6.82 (1H, d, J 8.3), 6.00 (2H, s), 5.74 (2H, s), 3.06 (3H, s).

### Example 15

### Preparation of N-(benzo[d][1,3]-dioxol-5-yl)-1-(4-(N,N-dimethylsulfamoyl)benzyl)-1H-indazole-3-carboxamide (Compound 128)

### 4-(Bromomethyl)-N,N-dimethylbenzenesulfonamide/4-(chloromethyl)-N,N-dimethylbenzenesulfonamide

A solution of dimethylamine (1 ml of 2M in THF) and DIPEA (0.265 mL, 2.1 mmol) in DCM (5 ml) was added drop-wise to a stirred solution of 4-(bromomethyl)benzene-1-sulfonyl chloride (539 mg, 2.00 mmol) in DCM (5 ml) at 0° C. After the addition was completed the resulting mixture was stirred for 16 h whilst warming up to room temperature. Another 0.2 ml of 2M dimethylamine in THF was then added in order to push the reaction the completion—reaction mixture was stirred for another 4 h. Solution was diluted with water, aq. layer back extracted with DCM, the combined organic extracts were washed with brine, dried (magnesium sulfate) and concentrated in vacuo. The residue was recrystalised with CHCl₃ (removing the insoluble impurities) to give 190 mg product (34% based on the bromo-product). ¹H NMR (CDCl₃): 7.71-7.64 (2H, br m), 7.53-7.46 (2H, br m), 4.56 (2H, s), 2.63 (3H, s).

### N-(Benzo[d][1,3]dioxol-5-yl)-1-(4-(N,N-dimethylsulfamoyl)benzyl)-1H-indazole-3-carboxamide (Compound 128)

The title compound was prepared from 4-(bromomethyl)-N,N-dimethylbenzenesulfonamide/4-(chloromethyl)-N,N-dimethylbenzenesulfonamide by subsequently following procedures B (yield 35%), C and P afford 69 mg (42%). (LCMS RT=2.49 min, MH⁺=478.7), ¹H NMR (CDCl₃): 8.63 (1H, br s), 8.38 (1H, d, J 8.2), 7.65 (1H, d, J 8.2), 7.40 (1H, d, J 2.1), 7.38-7.33 (1H, br m), 7.32-7.23 (4H, br m), 6.94 (1H, dd, J 8.3 & 2.2), 6.72 (1H, d, J 8.4), 5.89 (2H, s), 5.63 (2H, s), 2.60 (6H, s).

### Example 16

### Preparation of N-(Benzo[d][1,3]-dioxol-5-yl)-1)-1-(4-(diethylcarbamoyl)benzyl)-1H-indazole-3-carboxamide (Compound 151)

Procedures M (using diethylamine), B, C then D were used to afford 69 mg of the title compound (39% step M, 44% step B, 100% step C, 37% step D). LCMS RT=2.45 min, MH⁺=471.4. ¹H NMR (D₆-DMSO): 10.31 (1H, s), 8.25 (1H, d, J 8.1), 7.83 (1H, d, J 8.5), 7.54 (1H, d, J 2.0), 7.49 (1H, t, J 8.0), 7.37-7.27 (6H, m), 6.90 (1H, d, J 8.4), 6.01 (2H, s), 5.87 (2H, s), 3.12 (4H, m), 1.10-1.01 (6H, m).

### Example 17

### Preparation of N-(Benzo[d][1,3]dioxol-5-yl)-1-(4-(ethylsulfonyl)benzyl)-1H-indazole-3-carboxamide (Compound 156)

Procedure S: To a stirred solution of ethanethiol (1.31 mL, 17.7 mmol) in dimethylsulfoxide (10 mL) was added potassium carbonate (4.45 g, 32.2 mmol) and 4-fluorobenzaldehyde (2 g, 16.1 mmol). The reaction mixture was heated to 100° C. and stirred for 16 hours. The reaction mixture was then allowed to cool to room temperature and poured into water. The aqueous phase was extracted with ethyl acetate (×3) then the combined organic layers were washed (water then twice with brine), dried over sodium sulfate and concentrated in vacuo to afford 2.46 g (92%) of 4-(ethylthio)benzaldehyde. ¹H NMR (D₆-DMSO): 9.91 (1H, s), 7.81 (2H, d, J 8.3), 7.45 (2H, d, J 8.4), 3.10 (2H, q, J 7.4), 1.29 (3H, t, J 7.4).

Procedure T: To a stirred solution of 4-(ethylthio)benzaldehyde (2.46 g, 14.8 mmol) in dichloromethane (100 mL) at 0° C. was added m-CPBA (7 g, 31.1 mmol) in portions. The reaction mixture was stirred for 1 hour then 1M sodium hydroxide (35 mL) was added and stirred for a further 10 minutes. The reaction mixture was extracted with dichloromethane and the organic layer was washed with brine and concentrated in vacuo. ¹H NMR suggested that m-CPBA remained, so the crude product was dissolved in ethyl acetate, washed five times with 1M sodium hydroxide then concentrated in vacuo to give 2.16 g (74%) of 4-(ethylsulfonyl)benzaldehyde. ¹H NMR (D₆-DMSO): 10.20 (1H, s), 8.24-8.15 (4H, m), 3.44 (2H, q, J 7.4), 1.16 (3H, t, J 7.4).

Procedure U: To a stirred solution of 4-(ethylsulfonyl)benzaldehyde (1.7 g, 8.6 mmol) in IMS (40 mL) was added sodium tetraborohydride (650 mg, 17.2 mmol). The reaction mixture was stirred at room temperature for 2 hours, then neutralised with amberlite-H⁺resin, filtered and concentrated in vacuo to afford a quantitative yield of (4-(ethylsulfonyl)phenyl)methanol. ¹H NMR (CDCl₃): 7.88 (2H, d, J 8.3), 7.57 (2H, d, J 8.0), 4.84 (2H, s), 3.12 (2H, q, J 7.4), 1.28 (3H, t, J 7.4).

1.26 g (55%) of 1-(bromomethyl)-4-(ethylsulfonyl)benzene was obtained via procedure R.

Procedure V: A stirred solution of methyl 1H-indazole-3-carboxylate (0.92 g, 5.2 mmol), 1-(bromomethyl)-4-(ethylsulfonyl)benzene (1.37 g, 5.2 mmol) and cesium carbonate (1.69 g, 5.2 mmol) in anhydrous DMF was heated to 80° C. After 16 hours, the reaction mixture was allowed to cool to room temperature and was partitioned between ethyl acetate and water. The organic phase was washed three times with water then the combined aqueous layers were extracted with ethyl acetate. The combined organic layers were washed with brine, dried over MgSO₄ and concentrated in vacuo. The crude product was purified by column chromatography, eluting with ethyl acetate/petrol 0/1 to 4/1, v/v, to afford 824 mg (44%) of methyl 1-(4-(ethylsulfonyl)benzyl)-1H-indazole-3-carboxylate. ¹H NMR (CDCl₃): 8.30 (1H, d, J 8.5), 7.86 (2H, d, J 8.3), 7.49-7.43 (1H, m), 7.40-7.35 (4H, m), 5.81 (2H, s), 4.09 (3H, s), 3.09 (2H, q, J 7.4), 1.26 (3H, t, J 7.5).

Subsequent steps were carried out according to procedures C and D to afford 249 mg of the title compound (98% step C, 74% step D). LCMS RT=2.38, MH⁺=464.1. ¹H NMR (D₆-DMSO): 10.32 (1H, s), 8.26 (1H, d, J 8.1), 7.84 (3H, t, J 8.2), 7.54-7.47 (4H, m), 7.36-7.31 (2H, m), 6.90 (1H, d, J 8.4), 6.01 (2H, s), 5.97 (2H, s), 3.24 (2H, q, J 7.4), 1.05 (3H, t, J 7.4).

### Example 18

### Preparation of N-(Benzo[d][1,3]dioxol-5-yl)-1-(4-(isopropylsulfonyl)benzyl)-1H-indazole-3-carboxamide (Compound 152)

Procedures S (using propane-2-thiol, 90% yield), T (62%), U (100%), R (58%), V (40%), C (79%) and D (73%) were used to afford 245 mg of the title compound. LCMS RT=2.45 min, MH⁺=478.1. ¹H NMR (D₆-DMSO): 10.33 (1H, s), 8.26 (1H, d, J 8.1), 7.83 (3H, d, J 8.3), 7.54-7.46 (4H, m), 7.36-7.31 (2H, m), 6.90 (1H, d, J 8.4), 6.01 (1H, s), 5.98 (1H, s), 3.39 (1H, m), 1.11 (6H, d, J 6.8).

### Example 19

### Preparation of N-(Benzo[d][1,3]dioxol-5-yl)-1-((5-ethylpyridin-2-yl)methyl)-1H-indazole-3-carboxamide (Compound 153)

Procedures R (100%), B (72%), C (72%) then D (45%) were used to afford 137 mg of the title compound. LCMS RT=2.44, MH⁺=401.1. ¹H NMR (D₆-DMSO): 10.29 (1H, s), 8.37 (1H, d, J 1.7), 8.24 (1H, d, J 8.1), 7.76 (1H, d, J 8.5), 7.61 (1H, dd, J 8.0 & 2.3), 7.53 (1H, d, J 2.0), 7.50-7.44 (1H, m), 7.36-7.32 (2H, m), 7.05 (1H, d, J 8.0), 6.89 (1H, d, J 8.5), 6.00 (2H, s), 5.88 (2H, s), 2.57 (2H, q, J 7.6), 1.14 (3H, t, J 7.6).

### Example 20

### Preparation of N-(Benzo[d][1,3]-dioxol-5-yl)-1-(5-isopropyl-1,2,4-oxadiazol-3-yl)methyl)-1H-indazole-3-carboxamide (Compound 154)

Procedures R (56%), B (65%), C (76%) then D (46%) were used to afford 74 mg of the title compound. LCMS RT=2.50, MH⁺=405.9. ¹H NMR (D₆-DMSO): 10.32 (1H, s), 8.24 (1H, d, J 8.1), 7.84 (1H, d, J 8.5), 7.56-7.52 (2H, m), 7.38-7.33 (2H, m), 6.89 (1H, d, J 8.4), 6.01 (4H, s), 3.26 (1H, m), 1.26 (6H, d, J 7.0).

### Example 21

### Preparation of N-(benzo[d][1,3]dioxol-5-yl)-1-(5-ethyl-1,3,4-oxadiazol-2-yl)methyl)-1H-indazole-3-carboxamide (Compound 155)

Procedures B (80%), C (93%) then D (14%) were used to afford 59 mg of the title compound. LCMS RT=2.18 min, MH⁺=392.0. ¹H NMR (D₆-DMSO): 10.34 (1H, s), 8.24 (1H, d, J 8.1), 7.86 (1H, d, J 8.6), 7.59-7.53 (1H, m), 7.52 (1H, d, J 2.0), 7.37 (1H, t, J 7.6), 7.34 (1H, dd, J 8.5 & 2.1), 6.89 (1H, d, J 8.5), 5.14 (2H, s), 6.01 (2H, s), 2.81 (2H, q, J 7.6), 1.18 (3H, m).

The following compounds were prepared using previously described methods.

### N-(2,3-Dihydrobenzo[b][1,4]dioxin-6-yl)-5-fluoro-1-(4-(morpholine-4-carbonyl)benzyl)-1H-indazole-3-carboxamide (Compound 160)

Procedures A-D were used to prepare 301 mg of the title compound. LCMS RT=1.98 min, MH⁺=516.9. ¹H NMR (D₆-DMSO): 10.27 (1H, s), 7.90 (2H, m), 7.49-7.31 (7H, m), 6.82 (1H, d, J 8.8), 5.88 (2H, s), 4.23 (4H, m), 3.59-3.33 (8H, br m).

### N-(2,3-Dihydrobenzo[b][1,4]dioxin-6-yl)-1-(4-ethylbenzyl)-1H-indazole-3-carboxamide (Compound 158)

Procedures A-D were used to prepare 407 mg of the title compound. LCMS RT=2.58 min, MH⁺=413.9. ¹H NMR (D₆-DMSO): 10.20 (1H, s), 8.23 (1H, d, J 8.1), 7.80 (1H, d, J 8.5), 7.50 (1H, d, J 2.4), 7.46 (1H, m), 7.36-7.28 (2H, m), 7.19 (4H, m), 6.82 (1H, 8.8), 5.77 (2H, s), 4.24 (2H, q, J 4.8), 2.54 (2H, q, J 7.6), 1.12 (3H, t, J 7.6).

### 1-(4-Ethylbenzyl)-N-(2,2,3,3-tetrafluoro-2,3-dihydrobenzo[b][1,4]dioxin-6-yl)-1H-indazole-3-carboxamide (Compound 159)

Procedures A-D were used to prepare 597 mg of the title compound. LCMS RT=3.50 min, MH⁺=486.2. ¹H NMR (D₆-DMSO): 10.76 (1H, s), 8.24 (1H, d, J 8.1), 8.08 (1H, d, J 2.3), 7.84 (2H, m), 7.50 (2H, m), 7.34 (1H, t, J 7.7), 7.19 (4H, q, J 8.2), 5.80 (2H, s), 2.54 (2H, q, J 7.6), 1.12 (3H, t, J 7.6). 11-(4-(Morpholine-4-carbonyl)benzyl)-N-(2,2,3,3-tetrafluoro-2,3-dihydrobenzo[b][1,4]dioxin-6-yl)-1H-indazole-3-carboxamide (Compound 161)

Procedures A-D were used to prepare 137 mg of the title compound. Purification by preparative hplc at the final stage. LCMS RT=2.44 min, MH⁺=571.2. NMR (D₆-DMSO): 10.78 (1H, s), 8.26 (1H, d, J 8.2), 8.08 (1H, d J 2.3), 7.85 (2H, m), 7.50 (2H, m), 7.36 (5H, m), 5.90 (2H, s), 3.68-3.45 (8H, br m).

### N-(2,3-Dihydrobenzo[b][1,4]dioxin-6-yl)-6-fluoro-1-(4-(morpholine-4-carbonyl)benzyl)-1H-indazole-3-carboxamide (Compound 162)

Procedures A-D were used to prepare 120 mg of the title compound. Purification by preparative hplc at the final stage. LCMS RT=1.99 min, MH⁺=517.3. ¹H NMR (D₆-DMSO): 10.26 (1H, s), 8.24 (1H, q, J 4.8), 7.79 (1H, dd, J 2.2 & 9.6), 7.47 (1H, d, J 2.4), 7.35 (5H, m), 7.22 (1H, td, J 2.2 & 9.4), 6.82 (1H, d, J 8.8), 5.82 (2H,$), 4.30-4.18 (4H, br m), 3.69-3.44 (8H, br m).

### 1-(4-(Methylsulphonyl)benzyl)-1H-indazole-3-carboxylic acid (Compound 157)

The title compound was prepared according to the protocols A-C to afford 435 mg (45%). LCMS RT=1.55 min, MH⁺=330.8. ¹H NMR (D₆-DMSO): 13.16 (1H, br s), 8.11 (1H, d, J 8.2), 7.87 (3H, br t, J 8.1), 7.48 (3H, m), 7.34 (1H, m), 5.92 (2H, s), 3.17 (3H, s).

### Example 22

The potential activity of the compounds of formula (I) for use in the treatment of DMD may be demonstrated in the following predictive assay.

### Luciferase Reporter Assay (Murine H2K Cells)

The cell line used for the screen is an immortalized mdx mouse H2K cell line that has been stably transfected with a plasmid containing ≈5 kb fragment of the Utrophin A promoter including the first untranslated exon linked to a luciferase reporter gene.

Under conditions of low temperature and interferon containing media, the cells remain as myoblasts. These are plated into 96 well plates and cultured in the presence of compound for three days. The level of luciferase is then determined by cell lysis and reading of the light output from the expressed luciferase gene utilising a plate luminometer.

### Luciferase Assay for 96 Well Plates

The H2K/mdx/Utro A reporter cell line cells were plated out into 96 well plates (Falcon 353296, white opaque) at a density of approximately 5000 cells/well in 190 μl normal growth medium. The plates were then incubated at 33° C. in the presence of 10% CO₂ for 24 hrs.

Compounds were dosed by adding 10 μl of diluted compound to each well giving a final concentration of 10 μM (where a different final concentration was required, the amount of compound solution added was amended accordingly). The plates were then incubated for a further 48 hrs. Cells were then lysed in situ following the manufacture's protocols (Promega Steady-Glo Luciferase Assay System(E2520). Then counted for 10 seconds using a plate luminometer (Victor1420).

### Compound Storage

Compounds for screening were stored at −20° C. as 10 mM stocks in 100% DMSO until required.

### Results

Biological activity as assessed using the luciferase reporter assay in murine H2K cells, and the results are shown in Table 1, which also lists the concentration of the test compound solution in μM.

The results in Table 1 are classified as follows:

+ Up to 200% relative to control
++ Between 201% and 300% relative to control
+++ Between 301% and 400% relative to control
++++ Above 401% relative to control

TABLE 1

| Compound | | |
|---|---|---|
| No. | Activity | Concentration |
| 1 | + | 10 |
| 2 | + | 10 |
| 3 | + | 10 |
| 4 | +++ | 10 |
| 5 | + | 10 |
| 6 | +++ | 10 |
| 7 | + | 10 |
| 8 | + | 10 |
| 9 | + | 10 |
| 10 | ++ | 10 |
| 11 | + | 10 |
| 12 | + | 10 |
| 13 | + | 10 |
| 14 | +++ | 10 |
| 15 | + | 10 |
| 16 | ++++ | 10 |
| 17 | ++ | 10 |
| 18 | ++ | 10 |
| 19 | ++++ | 10 |
| 20 | + | 10 |
| 21 | ++++ | 10 |
| 22 | + | 10 |
| 23 | + | 10 |
| 24 | + | 10 |
| 25 | ++++ | 10 |
| 26 | + | 10 |
| 27 | + | 10 |
| 28 | ++ | 10 |
| 29 | + | 10 |
| 30 | + | 10 |
| 31 | + | 10 |
| 32 | + | 10 |
| 33 | + | 10 |
| 34 | +++ | 10 |
| 35 | + | 10 |
| 36 | + | 10 |
| 37 | + | 10 |
| 38 | ++ | 10 |
| 39 | + | 10 |
| 40 | + | 30 |
| 41 | + | 10 |
| 42 | ++++ | 10 |
| 43 | + | 10 |
| 44 | + | 10 |
| 45 | ++++ | 10 |
| 46 | + | 10 |
| 47 | + | 10 |
| 48 | + | 10 |
| 49 | + | 10 |
| 50 | + | 10 |
| 51 | + | 10 |
| 52 | + | 10 |
| 53 | + | 10 |
| 54 | + | 10 |
| 55 | + | 10 |
| 56 | + | 10 |
| 57 | + | 10 |
| 58 | ++ | 10 |
| 59 | + | 10 |
| 60 | + | 10 |
| 61 | ++++ | 10 |
| 62 | +++ | 10 |
| 63 | ++++ | 10 |
| 64 | + | 10 |
| 65 | ++ | 10 |
| 66 | + | 10 |
| 67 | + | 10 |
| 68 | + | 10 |
| 69 | + | 10 |
| 70 | + | 10 |
| 71 | + | 10 |
| 72 | + | 10 |
| 73 | + | 10 |
| 74 | + | 10 |
| 75 | ++ | 10 |
| 76 | + | 10 |
| 77 | + | 10 |
| 78 | + | 10 |
| 79 | + | 10 |
| 80 | + | 10 |
| 81 | + | 10 |
| 82 | + | 10 |
| 83 | + | 10 |
| 84 | +++ | 10 |
| 85 | + | 10 |
| 86 | + | 10 |
| 87 | + | 10 |
| 88 | + | 3 |
| 89 | ++ | 10 |
| 90 | + | 0.3 |
| 91 | + | 10 |
| 92 | + | 3 |
| 93 | + | 10 |
| 94 | + | 10 |
| 95 | + | 10 |
| 96 | + | 10 |
| 97 | + | 10 |
| 98 | + | 10 |
| 99 | ++ | 10 |
| 100 | + | 10 |
| 101 | + | 10 |
| 102 | ++ | 10 |
| 103 | + | 3 |
| 104 | +++ | 10 |
| 105 | ++++ | 10 |
| 106 | ++ | 10 |
| 107 | ++ | 10 |
| 108 | ++++ | 10 |
| 109 | + | 10 |
| 110 | +++ | 10 |
| 111 | + | 10 |
| 112 | ++ | 10 |
| 113 | + | 10 |
| 114 | ++ | 10 |
| 115 | + | 10 |
| 116 | ++ | 10 |
| 117 | ++ | 10 |
| 118 | + | 0.3 |
| 119 | + | 10 |
| 120 | +++ | 10 |
| 121 | ++ | 10 |
| 122 | ++ | 10 |
| 123 | ++ | 10 |
| 124 | +++ | 10 |
| 125 | + | 10 |
| 126 | + | 10 |
| 127 | + | 10 |
| 128 | ++ | 10 |
| 129 | + | 10 |
| 130 | ++ | 10 |
| 131 | + | 10 |
| 132 | ++ | 10 |
| 133 | + | 10 |
| 134 | + | 10 |
| 135 | ++ | 10 |
| 136 | + | 10 |
| 137 | ++ | 10 |
| 138 | ++ | 10 |
| 139 | + | 10 |
| 140 | +++ | 10 |
| 141 | + | 0.3 |
| 142 | ++ | 10 |
| 143 | +++ | 10 |
| 144 | +++ | 10 |
| 145 | + | 10 |
| 146 | + | 10 |
| 147 | + | 10 |
| 148 | + | 30 |
| 149 | + | 0.1 |
| 150 | + | 1 |
| 151 | + | 10 |
| 152 | + | 10 |
| 153 | + | 10 |
| 154 | + | 10 |
| 155 | + | 10 |
| 156 | ++ | 10 |
| 157 | + | 10 |
| 158 | ++ | 10 |
| 159 | + | 10 |
| 160 | + | 10 |
| 161 | ++ | 10 |
| 162 | ++ | 10 |
| 163 | + | 10 |

The results in Table 1 show that all of the exemplified compounds had increased activity in the luciferase reporter assay relative to the control.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims.

All publications, patents, and patent applications cited in this specification are incorporated herein by reference as if each such publication, patent or patent application were specifically and individually indicated to be incorporated herein by reference.

1. A method for the therapeutic or prophylactic treatment of (a) when X is N, Duchenne muscular dystrophy, Becker muscular dystrophy, or cachexia, or (b) when X is CR^{x}, Duchenne muscular dystrophy or Becker muscular dystrophy, comprising administering a compound of formula (I): 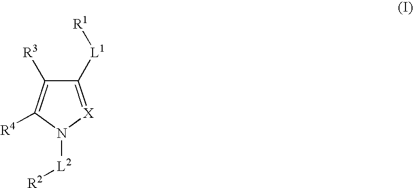
or a tautomer, enantiomer, pharmaceutically acceptable salt, hydrate, solvate, complex, or prodrug thereof, wherein
X is CR^{x} or N;
R^{x} is H or C₁-C₆ alkyl;
L¹ is a bond, —(CR⁵R⁵)ₙ—, —NR⁵—, —O—, —S—, —(CR⁵R⁵)ₙNR⁵—, —C(O)NR⁵—, —C(O)NR⁵O—, —C(S)NR⁵—, —NR⁵C(O)—, —NR⁵C(S)—, —NR⁵C(NH)—, —SO₂NR⁵—, —NR⁵SO₂—, —C(O)—, —C(S)—, —SO—, —SO₂—, —CR⁵═CR⁵—, —C≡C—, —NR⁵C(O)NR⁵—, —NR⁵C(S)NR⁵—, —NR⁵C(NH)NR⁵—, —NR⁵C(O)O—, or —OC(O)NR⁵—;
R¹ is C₁-C₁₀ alkyl, C₂-C₆ alkenyl, or C₂-C₆ alkynyl, each of which is optionally substituted with one or more OR^{S}, N(R⁵)₂, R⁶, or OR⁶; or
R¹ is an optionally substituted 5-10 membered mono- or bicyclic aromatic or 4-7 membered non-aromatic carbocyclic or heterocyclic ring system;
L² is —(CR⁵R⁵)ₙ—, —(CR⁵R⁵)ₙO—, —C(O)—, —C(NR⁵)—, —SO₂—, —C(O)NR⁵—, or —SO₂NR⁵—;
R² is C₁-C₁₀ alkyl, C₂-C₆ alkenyl, or C₂-C₆ alkynyl, each of which is optionally substituted with optionally substituted 5-10 membered mono- or bicyclic aromatic or 4-7 membered non-aromatic carbocyclic or heterocyclic ring system; or
R² is an optionally substituted 5-10 membered mono- or bicyclic aromatic or 4-7 membered non-aromatic carbocyclic or heterocyclic ring system;
Each of R³ and R⁴ is independently hydrogen, C₁-C₆ alkyl, OH, —O(C₁-C₆ alkyl), halo, SOₘR⁵, or NR⁵R⁵; or
R³ and R⁴ together with the carbon atoms to which they are attached form a 5-6 membered aromatic or 5-7 membered non-aromatic carbocyclic or heterocyclic ring system, each of which is optionally substituted with one or more C₁-C₆ alkyl, OH, —O(C₁-C₆ alkyl), halo, SOₘR⁵, C(O)R⁵, or NR⁵R⁵;
m is 0, 1, or 2;
n is 1 or 2;
each R⁵ is independently H or C₁-C₆ alkyl optionally substituted with one or more halo; and
R⁶ is an optionally substituted 5-10 membered mono- or bicyclic aromatic or 4-7 membered non-aromatic carbocyclic or heterocyclic ring system.

2. The method of claim 1, wherein L¹ is a bond, —CH₂—, —CH₂CH₂—, —C(O)NR⁵—, —C(S)NR⁵—, —NR⁵—C(O)—NR⁵—, —NR⁵C(O)—, —C(O)—, —NR⁵—SO₂—, —C(O)NR⁵—O—, —S—, —SO₂—, or —CH₂NR⁵—; where R⁵ is H or C₁-C₄ alkyl.

3-4. (canceled)

5. The method of claim 1, wherein R¹ is a 5-10 membered mono- or bicyclic aromatic or 4-7 membered non-aromatic carbocyclic or heterocyclic ring system, each of which is unsubstituted or substituted with one or more R⁵, R⁶, —C(O)NR⁵R⁵, C(O)NHR⁶, —NR⁵C(O)R⁵, —NR⁵C(O)R⁶, —C(O)OR⁵, —C(O)OR⁶, —C(O)R⁵, —C(O)R⁶, —(CH₂)_{q}OR⁵, —(CH₂)_{q}OR⁶, —SO₂R⁵, —SO₂R⁶, halo, or CN, wherein q is 0, 1, or 2.

6. The method of claim 5, wherein R¹ is (i) phenyl, pyridine, pyrimidine, pyridazine, pyrrole, furan, thiophene, benzofuran, benzothiazole, benzodioxolane, benzodioxyl, benzodioxane, thiadiazole, isoxazole, cyclopropyl, cyclobutyl, piperazine, pyrrolidine, pyrrolidinone, piperidine, piperazine, morpholine, thiazole, naphthalene, quinoxaline, quinoline, benzoxazole, indane, or tetrahydronaphthalene, each of which is optionally substituted with one or more CONH₂, CON(CH₃)₂, CONH(C₁-C₃ alkyl), CO(C₁-C₃ alkyl), C(O)heterocyclyl, COOH, COO(C₁-C₃ alkyl), SO₂CH₃, CH₂CH₂OH, O(C₁-C₃ alkyl), —NHC(O)(C₁-C₃ alkyl), heterocyclyl, phenoxy, C₁-C₃ alkyl, (CH₂)_{q}OH, (CH₂)_{q}O-phenyl, cyano, or halo; or
(ii) R¹ is

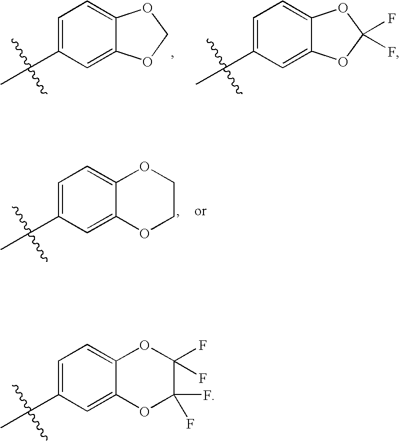

7-8. (canceled)

9. The method of claim 1, wherein R¹ is —(CH₂)—R⁶ or —(CH₂)₂—R⁶, wherein R⁶ is phenyl, furan, or tetrahydrofuran, each of which is optionally substituted with one or more C₁-C₃ alkyl, O(C₁-C₃ alkyl), or halo.

10. The method of claim 1, wherein L² is —(CH₂)ᵣ—, —(CH₂)ᵣO—, —C(O)— or SO₂—, wherein r is 1, 2 or 3.

11-12. (canceled)

13. The method of claim 1, wherein R² is an optionally substituted 5- or 6-membered aromatic or non-aromatic cyclic group.

14. The use method of claim 13, wherein R² is phenyl, pyridine, piperidine, cyclohexyl, pyrimidine, thiophene, isoxazole, or oxadiazole, each of which is optionally substituted with one or more halo, cyano, R⁷, C(O)NR⁵R⁷, —C(O)R⁷, —SO₂NR⁵R⁷, —SO₂N(R⁵)(OR⁷), —(CR⁵R⁵)_{q}R⁷, —SO₂R⁷, —(CR⁵R⁵)_{q}OR⁷ or —O(CR⁵R⁵)₉R⁷; wherein
R⁵ is H or C₁-C₄ alkyl;
R⁷ is (i) H or C₁-C₆ alkyl, cycloalkyl, heterocyclyl, aryl, aralkyl, or heteroaryl, wherein the alkyl, cycloalkyl, heterocyclyl, aryl, aralkyl, and heteroaryl are each optionally substituted with one or more halo or —O(C₁-C₆ alkyl); and wherein the cycloalkyl, heterocyclyl, aryl, aralkyl, and heteroaryl are each optionally substituted with one or more C₁-C₆ alkyl; or (ii) C₁-C₄ alkyl, C₁-C₄ alkenyl, or C₁-C₄ alkynyl; or (iii) methyl, trifluoromethyl, ethyl, propyl, isopropyl, t-butyl, 2-propenyl, or 2-propynyl; or (iv) phenyl, benzodioxolane, pyrrolidine, morpholine, piperidine, or pyrrolidinedione; or
when R⁵ and R⁷ are attached to the same nitrogen atom, R⁵ and R⁷ may be combined with that nitrogen atom to form a 4 to 7 membered saturated or unsaturated ring system which may contain one or more additional heteroatoms.

15. The method of claim 1, wherein R² is naphthalene or benzodioxolane, each of which is optionally substituted with one or more halo, cyano, R⁷, C(O)NR⁵R⁷, —C(O)R⁷, —SO₂NR⁵R⁷, —SO₂N(R⁵)(OR⁷), —(CR⁵R⁵)_{q}R⁷, —SO₂R⁷, —(CR⁵R⁵)_{q}OR⁷ or —O(CR⁵R⁵)_{q}R⁷; wherein
R⁵ is H or C₁-C₄ alkyl;
R⁷ is (i) H or C₁-C₆ alkyl, cycloalkyl, heterocyclyl, aryl, aralkyl, or heteroaryl, wherein the alkyl, cycloalkyl, heterocyclyl, aryl, aralkyl, and heteroaryl are each optionally substituted with one or more halo or —O(C₁-C₆ alkyl); and wherein the cycloalkyl, heterocyclyl, aryl, aralkyl, and heteroaryl are each optionally substituted with one or more C₁-C₆ alkyl; or (ii) alkyl, C₁-C₄ alkenyl, or C₁-C₄ alkynyl; or (iii) methyl, trifluoromethyl, ethyl, propyl, isopropyl, t-butyl, 2-propenyl, or 2-propynyl; or (iv) phenyl, benzodioxolane, pyrrolidine, morpholine, piperidine, or pyrrolidinedione; or
when R⁵ and R⁷ are attached to the same nitrogen atom, R⁵ and R⁷ may be combined with that nitrogen atom to form a 4 to 7 membered saturated or unsaturated ring system which may contain one or more additional heteroatoms.

16-20. (canceled)

21. The method of claim 1, wherein X is N.

22. The method of claim 1, wherein R³ and R⁴ are independently hydrogen or C₁-C₄ alkyl.

23. The method of claim 1, wherein R³ and R⁴ combine to form an optionally substituted fused ring system.

24. The method of claim 23, wherein R³ and R⁴ combine to form optionally substituted benzene, cyclohexenyl, or cyclopentenyl ring.

25. (canceled)

26. The method of claim 24, or a tautomer, enantiomer or pharmaceutically acceptable salt, hydrate, solvate, complex or prodrug thereof, having formula (Ia): 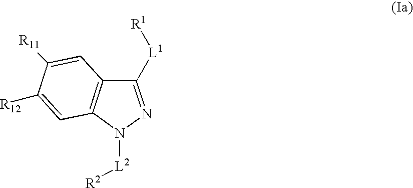
wherein
R¹¹ and R¹² are each independently H, C₁-C₆ alkyl, —O(C₁-C₆ alkyl), or halo.

27. The method of claim 26, wherein (i) R¹¹ is H and R¹² is H; or (ii) R¹¹ is H and R¹² is F; or (iii) R¹² is H and R¹¹ is F, CF₃, OMe, or methyl.

28-29. (canceled)

30. The method of claim 1, wherein the compound is:
N-(benzo[d]thiazol-2-yl)-1-(4-(N-methoxy-N-methylsulfamoyl)benzyl)-1H-indazole-3-carboxamide;
1-(1-(4-((benzo[d][1,3]dioxol-5-yloxy)methyl)benzyl)-5-(trifluoromethyl)-1H-indazol-3-yl)-3-(2,5-dimethoxyphenyl)urea;
1-(4-(methylsulfonyl)benzyl)-N-(thiazol-2-yl)-1H-indazole-3-carboxamide;
N-(benzo[d][1,3]dioxol-5-yl)-1-(4-(methylsulfonyl)benzyl)-1H-indazole-3-carboxamide;
N-(4-methoxyphenyl)-1-(3-phenoxypropyl)-1H-indazole-3-carboxamide;
1-(4-(N-methyl-N-(prop-2-ynyl)sulfamoyl)benzyl)-N-(4-(piperidin-1-yl)phenyl)-1H-indazole-3-carboxamide;
N-(benzo[d]thiazol-2-yl)-1-(4-(N,N-diethylsulfamoyl)benzyl)-1H-indazole-3-carboxamide;
5-methoxy-1-(4-(N-methoxy-N-methylsulfamoyl)benzyl)-N-(4-(piperidin-1-yl)phenyl)-1H-indazole-3-carboxamide;
1-(1-(4-((benzo[d][1,3]dioxol-5-yloxy)methyl)benzyl)-5-(trifluoromethyl)-1H-indazol-3-yl)-3-(3-fluorophenyl)urea;
1-(4-(N,N-diethylsulfamoyl)benzyl)-N-(4-(piperidin-1-yl)phenyl)-1H-indazole-3-carboxamide;
Methyl 4-(3-(1-(4-((3-methoxyphenoxy)methyl)benzyl)-1H-indazol-3-yl)ureido) benzoate;
1-(2,6-dimethoxyphenyl)-3-(1-(4-(trifluoromethyl)benzyl)-1H-indazol-3-yl)urea;
1-(1-((6-chlorobenzo[d][1,3]dioxol-5-yl)methyl)-5-methyl-1H-indazol-3-yl)-3-(2-isopropylphenyl)urea;
1-(3-phenoxypropyl)-N-(4-(piperidin-1-yl)phenyl)-1H-indazole-3-carboxamide;
1-m-tolyl-3-(1-(4-(trifluoromethyl)benzyl)-1H-indazol-3-yl)urea;
1-(3-phenoxypropyl)-N-(thiazol-2-yl)-1H-indazole-3-carboxamide;
N-(benzo[d]thiazol-2-yl)-1-(4-(morpholine-4-carbonyl)benzyl)-1H-indazole-3-carboxamide;
1-(4-(methylsulfonyl)benzyl)-N-(4-(piperidin-1-yl)phenyl)-1H-indazole-3-carboxamide;
N-(4-isopropylphenyl)-1-(4-(morpholine-4-carbonyl)benzyl)-1H-indazole-3-carboxamide;
1-(4-(benzyl(methyl)carbamoyl)benzyl)-N-(4-(trifluoromethyl)phenyl)-1H-indazole-3-carboxamide;
N-(benzo[d][1,3]dioxol-5-yl)-1-(4-(N-methyl-N-(prop-2-ynyl)sulfamoyl)benzyl)-1H-indazole-3-carboxamide;
1-(4-(benzyl(methyl)carbamoyl)benzyl)-N-(4-isopropylphenyl)-1H-indazole-3-carboxamide;
1-(4-(methylsulfonyl)benzyl)-N-(4-(trifluoromethoxy)phenyl)-1H-indazole-3-carboxamide;
N-(benzyloxy)-1-(4-(N-methyl-N-phenylsulfamoyl)benzyl)-1H-indazole-3-carboxamide;
N-(2,2-difluorobenzo[d][1,3]dioxol-5-yl)-1-(4-(morpholine-4-carbonyl)benzyl)-1H-indazole-3-carboxamide;
N-(4-isopropylphenyl)-1-(2-phenoxyethyl)-1H-indazole-3-carboxamide;
1-(biphenyl-4-ylmethyl)-N-(4-isopropylphenyl)-1H-indazole-3-carboxamide;
1-(2-fluorobenzyl)-3-(1-(4-((3-methoxyphenoxy)methyl)benzyl)-1H-indazol-3-yl)urea;
1-(1-(4-((benzo[d][1,3]dioxol-5-yloxy)methyl)benzyl)-5-(trifluoromethyl)-1H-indazol-3-yl)-3-(2-(trifluoromethyl)phenyl)urea;
1-(2,3-dihydro-1H-inden-5-yl)-3-(1-(4-((3-methoxyphenoxy)methyl)benzyl)-1H-indazol-3-yl)urea;
1-(1-(4-((benzo[d][1,3]dioxol-5-yloxy)methyl)benzyl)-5-(trifluoromethyl)-1H-indazol-3-yl)-3-(2,4-dimethoxyphenyl)urea;
1-(1-((6-chlorobenzo[d][1,3]dioxol-5-yl)methyl)-1H-indazol-3-yl)-3-(3-methylbenzyl)urea;
N-(1-(4-((2,5-dioxopyrrolidin-1-yl)methyl)benzyl)-1H-indazol-3-yl)quinoxaline-2-carboxamide;
N-(4-methoxyphenyl)-1-(4-(methylsulfonyl)benzyl)-1H-indazole-3-carboxamide;
N-(2,2-difluorobenzo[d][1,3]dioxol-5-yl)-1-(4-(methylsulfonyl)benzyl)-1H-indazole-3-carboxamide;
1-(4-(N-methoxy-N-methylsulfamoyl)benzyl)-N-(thiazol-2-yl)-1H-indazole-3-carboxamide;
1-(1-((6-chlorobenzo[d][1,3]dioxol-5-yl)methyl)-1H-indazol-3-yl)-3-(4-methoxyphenethyl)urea;
N-(4-carbamoylphenyl)-1-(4-(morpholine-4-carbonyl)benzyl)-1H-indazole-3-carboxamide;
1-(4-(N,N-diethylsulfamoyl)benzyl)-N-(pyridin-2-yl)-1H-indazole-3-carboxamide;
1-(2-(trifluoromethoxy)phenyl)-3-(1-(4-(trifluoromethyl)benzyl)-1H-indazol-3-yl)urea;
1-(4-(N,N-diethylsulfamoyl)benzyl)-N-(2-methyl-1,3-dioxoisoindolin-5-yl)-1H-indazole-3-carboxamide;
N-(benzo[d][1,3]dioxol-5-yl)-1-(4-tert-butylbenzyl)-1H-indazole-3-carboxamide;
1-(2-(4-fluorophenoxy)ethyl)-N-(4-(piperidin-1-yl)phenyl)-1H-indazole-3-carboxamide;
1-(2-methoxybenzyl)-3-(1-(4-((3-methoxyphenoxy)methyl)benzyl)-1H-indazol-3-yl)urea;
N-(benzo[d][1,3]dioxol-5-yl)-1-(3-phenoxypropyl)-1H-indazole-3-carboxamide;
N-(benzo[d][1,3]dioxol-5-yl)-1-(2-(4-fluorophenoxy)ethyl)-1H-indazole-3-carboxamide;
1-(1-((6-chlorobenzo[d][1,3]dioxol-5-yl)methyl)-1H-indazol-3-yl)-3-(2-fluorobenzyl)urea;
1-(4-tert-butylbenzyl)-N-(2,2-difluorobenzo[d][1,3]dioxol-5-yl)-1H-indazole-3-carboxamide;
1-(4-(N-methoxy-N-methylsulfamoyl)benzyl)-N-(pyridin-2-yl)-1H-indazole-3-carboxamide;
N-(benzo[d][1,3]dioxol-5-yl)-1-(biphenyl-4-ylmethyl)-1H-indazole-3-carboxamide;
1-(1-(4-((benzo[d][1,3]dioxol-5-yloxy)methyl)benzyl)-5-(trifluoromethyl)-1H-indazol-3-yl)-3-m-tolylurea;
1-(1-((6-chlorobenzo[d][1,3]dioxol-5-yl)methyl)-1H-indazol-3-yl)-3-(3,5-dimethoxyphenyl)urea;
1-(2,3-dihydro-1H-inden-5-yl)-3-(1-(4-(trifluoromethyl)benzyl)-1H-indazol-3-yl)urea;
1-(3,4-dimethoxyphenyl)-3-(1-(4-(3-methoxyphenoxy)methyl)benzyl)-1H-indazol-3-yl)urea;
1-(1-((6-chlorobenzo[d][1,3]dioxol-5-yl)methyl)-1H-indazol-3-yl)-3-(furan-2-ylmethyl)urea;
N-(4-methoxyphenyl)-1-(2-phenoxyethyl)-1H-indazole-3-carboxamide;
1-(1-(4-((benzo[d][1,3]dioxol-5-yloxy)methyl)benzyl)-5-(trifluoromethyl)-1H-indazol-3-yl)-3-p-tolylurea;
N-(benzo[d][1,3]dioxol-5-yl)-1-(2-phenoxyethyl)-1H-indazole-3-carboxamide;
1-p-tolyl-3-(1-(4-(trifluoromethyl)benzyl)-1H-indazol-3-yl)urea;
1-(2-(4-fluorophenoxy)ethyl)-N-(4-isopropylphenyl)-1H-indazole-3-carboxamide;
1-(4-(morpholine-4-carbonyl)benzyl)-N-(4-(trifluoromethoxy)phenyl)-1H-indazole-3-carboxamide;
N-(4-isopropylphenyl)-1-(4-(methylsulfonyl)benzyl)-1H-indazole-3-carboxamide;
N-(benzo[d][1,3]dioxol-5-yl)-1-(4-(morpholine-4-carbonyl)benzyl)-1H-indazole-3-carboxamide;
N-(4-carbamoylphenyl)-1-(4-(morpholinosulfonyl)benzyl)-1H-indazole-3-carboxamide;
1-(4-tert-butylbenzyl)-N-(4-methoxyphenyl)-1H-indazole-3-carboxamide;
N-(4-isopropylphenyl)-1-(4-(N-methyl-N-(prop-2-ynyl)sulfamoyl)benzyl)-1H-indazole-3-carboxamide;
1-(2-(4-fluorophenoxy)ethyl)-N-(pyridin-2-yl)-1H-indazole-3-carboxamide;
1-(1-(4-(3-methoxyphenoxy)methyl)benzyl)-1H-indazol-3-yl)-3-(2-(trifluoromethoxy)phenyl)urea;
1-(biphenyl-4-ylmethyl)-N-(4-(piperidin-1-yl)phenyl)-1H-indazole-3-carboxamide;
1-(4-tert-butylbenzyl)-N-(4-(trifluoromethoxy)phenyl)-1H-indazole-3-carboxamide;
1-(2-phenoxyethyl)-N-(thiazol-2-yl)-1H-indazole-3-carboxamide;
1-(4-(morpholine-4-carbonyl)benzyl)-N-((tetrahydrofuran-2-yl)methyl)-1H-indazole-3-carboxamide;
1-(3,4-dimethoxyphenethyl)-3-(1-(4-(trifluoromethyl)benzyl)-1H-indazol-3-yl)urea;
1-(biphenyl-4-ylmethyl)-N-(4-methoxyphenyl)-1H-indazole-3-carboxamide;
1-(2-phenoxyethyl)-N-(4-(piperidin-1-yl)phenyl)-1H-indazole-3-carboxamide;
N-(4-carbamoylphenyl)-1-(4-(N-methyl-N-(prop-2-ynyl)sulfamoyl)benzyl)-1H-indazole-3-carboxamide;
1-(4-(methylsulfonyl)benzyl)-N-(pyridin-2-yl)-1H-indazole-3-carboxamide;
1-(1-((6-chlorobenzo[d][1,3]dioxol-5-yl)methyl)-1H-indazol-3-yl)-3-(2-methoxybenzyl)urea;
1-(4-tert-butylbenzyl)-N-(4-(piperidin-1-yl)phenyl)-1H-indazole-3-carboxamide;
1-(4-methoxyphenethyl)-3-(1-(4-((3-methoxyphenoxy)methyl)benzyl)-1H-indazol-3-yl)urea;
N-(4-tert-butylphenyl)-1-(4-(methylsulfonyl)benzyl)-1H-indazole-3-carboxamide;
N-(3,4-dimethoxyphenyl)-1-(4-(methylsulfonyl)benzyl)-1H-indazole-3-carboxamide;
N-phenyl-1-(4-(methylsulfonyl)benzyl)-1H-indazole-3-carboxamide;
N-(4-ethylphenyl)-1-(4-(methylsulfonyl)benzyl)-1H-indazole-3-carboxamide;
N-(2-isopropylphenyl)-1-(4-(methylsulfonyl)benzyl)-1H-indazole-3-carboxamide;
N-(3-methoxyphenyl)-1-(4-(methylsulfonyl)benzyl)-1H-indazole-3-carboxamide;
N-(benzo[d][1,3]dioxol-5-yl)-1-benzyl-1H-indazole-3-carboxamide;
N-(4-hydroxyphenyl)-1-(4-(methylsulfonyl)benzyl)-1H-indazole-3-carboxamide;
N-(4-chlorophenyl)-1-(4-(methylsulfonyl)benzyl)-1H-indazole-3-carboxamide;
N-((1-(4-(methylsulfonyl)benzyl)-1H-indazol-3-yl)methyl)benzo[d][1,3]dioxol-5-amine;
1-(4-isopropylbenzyl)-N-(4-(methylsulfonyl)phenyl)-1H-indazole-3-carboxamide;
N-(benzo[d][1,3]dioxol-5-yl)-1-(4-(morpholine-4-carbonyl)phenethyl)-1H-indazole-3-carboxamide;
N-(benzo[d][1,3]dioxol-5-yl)-1-(4-(piperidine-1-carbonyl)benzyl)-1H-indazole-3-carboxamide;
N-(4-fluorophenyl)-1-(4-(methylsulfonyl)benzyl)-1H-indazole-3-carboxamide;
6-chloro-2-(1-(4-(methylsulfonyl)benzyl)-1H-indazol-3-yl)benzo[d]oxazole;
N-(3-isopropylphenyl)-1-(4-(methylsulfonyl)benzyl)-1H-indazole-3-carboxamide;
N-(benzo[d][1,3]dioxol-5-yl)-1-(pyridin-4-ylmethyl)-1H-indazole-3-carboxamide;
N-(3-isopropyl-1,2,4-thiadiazol-5-yl)-1-(4-(methylsulfonyl)benzyl)-1H-indazole-3-carboxamide;
1-(4-(methylsulfonyl)benzyl)-N-(quinolin-6-yl)-1H-indazole-3-carboxamide;
N-(benzo[d][1,3]dioxol-5-yl)-1-(3-(morpholine-4-carbonyl)benzyl)-1H-indazole-3-carboxamide;
1-(4-(methylsulfonyl)benzyl)-N-(4-(methylsulfonyl)phenyl)-1H-indazole-3-carboxamide;
N-(benzo[d][1,3]dioxol-5-yl)-1-(4-(methylsulfonyl)benzyl)-1H-indazole-3-carbothioamide;
1-(4-(methylsulfonyl)benzyl)-N-(quinolin-3-yl)-1H-indazole-3-carboxamide;
N-(3,4-dimethylphenyl)-1-(4-(methylsulfonyl)benzyl)-1H-indazole-3-carboxamide;
N-(2,3-dihydrobenzo[b][1,4]dioxin-6-yl)-1-(4-(methylsulfonyl)benzyl)-1H-indazole-3-carboxamide;
N-(benzo[d][1,3]dioxol-5-yl)-1-(pyridin-3-ylmethyl)-1H-indazole-3-carboxamide;
N-(4-acetamidophenyl)-1-(4-(methylsulfonyl)benzyl)-1H-indazole-3-carboxamide;
1-(4-(methylsulfonyl)benzyl)-N-p-tolyl-1H-indazole-3-carboxamide;
N-(4-acetamidophenyl)-1-(4-(trifluoromethyl)benzyl)-1H-indazole-3-carboxamide;
1-(4-(methylsulfonyl)benzyl)-N-(quinoxalin-6-yl)-1H-indazole-3-carboxamide;
N-(3,4-dichlorophenyl)-1-(4-(methylsulfonyl)benzyl)-1H-indazole-3-carboxamide;
1-(4-(methylsulfonyl)benzyl)-N-(4-propylphenyl)-1H-indazole-3-carboxamide;
N-(1,3-dihydrobenzo[c]thiophen-2,2-dioxy-5-yl)-1-(4-(methylsulfonyl)benzyl)-1H-indazole-3-carboxamide;
N-(1-(4-(methylsulfonyl)benzyl)-1H-indazol-3-yl)benzo[d][1,3]dioxole-5-carboxamide;
N-(benzo[d][1,3]dioxol-5-yl)-1-(4-(trifluoromethyl)benzyl)-1H-indazole-3-carboxamide;
1-(4-(methylsulfonyl)benzyl)-N-(naphthalen-2-yl)-1H-indazole-3-carboxamide;
N-(benzo[d][1,3]dioxol-5-yl)-1-(4-(dimethylcarbamoyl)benzyl)-1H-indazole-3-carboxamide;
1-(4-chlorophenyl)-3-(1-(4-(methylsulfonyl)benzyl)-1H-indazol-3-yl)urea;
N-(benzo[d][1,3]dioxol-5-yl)-1-(4-isopropylbenzyl)-1H-indazole-3-carboxamide;
N-(benzofuran-5-yl)-1-(4-(methylsulfonyl)benzyl)-1H-indazole-3-carboxamide;
1-(4-(methylsulfonyl)benzyl)-N-(5,6,7,8-tetrahydronaphthalen-2-yl)-1H-indazole-3-carboxamide;
N-(4-cyclohexylphenyl)-1-(4-(methylsulfonyl)benzyl)-1H-indazole-3-carboxamide;
N-(benzo[d][1,3]dioxol-5-yl)-1-(benzo[d][1,3]dioxol-5-ylmethyl)-1H-indazole-3-carboxamide;
N-(benzo[d][1,3]dioxol-5-yl)-1-(4-tert-butylbenzoyl)-1H-indazole-3-carboxamide;
1-(4-(methylsulfonyl)benzyl)-N-(5-methylthiazol-2-yl)-1H-indazole-3-carboxamide;
1-(4-(methylsulfonyl)benzyl)-N-(4-methylthiazol-2-yl)-1H-indazole-3-carboxamide;
N-(benzo[d][1,3]dioxol-5-yl)-1-(3-(methylsulfonyl)benzyl)-1H-indazole-3-carboxamide;
N-(benzo[d][1,3]dioxol-5-yl)-1-(4-(N,N-dimethylsulfamoyl)benzyl)-1H-indazole-3-carboxamide;
N-(benzo[d][1,3]dioxol-5-yl)-1-(thiophen-3-ylmethyl)-1H-indazole-3-carboxamide;
N-(2,3-dihydro-1H-inden-5-yl)-1-(4-(methylsulfonyl)benzyl)-1H-indazole-3-carboxamide;
N-(benzo[d][1,3]dioxol-5-yl)-1-(4-methylbenzyl)-1H-indazole-3-carboxamide;
N-(benzo[d][1,3]dioxol-5-yl)-1-(4-ethylbenzyl)-1H-indazole-3-carboxamide;
N-(6-methylpyridin-3-yl)-1-(4-(methylsulfonyl)benzyl)-1H-indazole-3-carboxamide;
N-(5-methylpyridin-2-yl)-1-(4-(methylsulfonyl)benzyl)-1H-indazole-3-carboxamide;
N-(2,2-difluorobenzo[d][1,3]dioxol-5-yl)-2-(3-(dimethylcarbamoyl)benzyl)-2H-indazole-3-carboxamide;
N-(benzo[d][1,3]dioxol-5-yl)-1-(2-(methylsulfonyl)benzyl)-1H-indazole-3-carboxamide;
N-(benzo[d][1,3]dioxol-5-yl)-1-(4-(morpholinosulfonyl)benzyl)-1H-indazole-3-carboxamide;
1-(4-(dimethylcarbamoyl)benzyl)-N-p-tolyl-1H-indazole-3-carboxamide;
1-(4-(dimethylcarbamoyl)benzyl)-N-(4-isopropylphenyl)-1H-indazole-3-carboxamide;
N-(benzo[d][1,3]dioxol-5-yl)-1-(4-(methylsulfonyl)benzoyl)-1H-indazole-3-carboxamide;
N-(benzo[d][1,3]dioxol-5-yl)-1-(naphthalen-2-ylmethyl)-1H-indazole-3-carboxamide;
N-(benzo[d][1,3]dioxol-5-yl)-1-(4-(pyrrolidine-1-carbonyl)benzyl)-1H-indazole-3-carboxamide;
N-(2,3-dihydrobenzo[b][1,4]dioxin-6-yl)-1-(4-(morpholine-4-carbonyl)benzyl)-1H-indazole-3-carboxamide;
N-(4-methoxyphenyl)-1-(4-(morpholine-4-carbonyl)benzyl)-1H-indazole-3-carboxamide;
N-(benzo[d][1,3]dioxol-5-yl)-1-(isoxazol-3-ylmethyl)-1H-indazole-3-carboxamide;
N-(benzo[d][1,3]dioxol-5-yl)-1-(4-(methylsulfonyl)benzyl)-1H-indole-3-carboxamide;
N-(2,4-dichlorophenyl)-1-ethyl-1H-pyrazole-3-carboxamide;
N-(benzo[d][1,3]dioxol-5-yl)-1-(4-(methylsulfonyl)benzyl)-1H-pyrazole-3-carboxamide;
N-(benzo[d][1,3]dioxol-5-yl)-1-(4-(methylsulfonyl)benzyl)-4,5,6,7-tetrahydro-1H-indazole-3-carboxamide;
N-(benzo[d][1,3]dioxol-5-yl)-1-(4-(methylsulfonyl)benzyl)-1,4,5,6-tetrahydrocyclopenta[c]pyrazole-3-carboxamide;
N-(benzo[d][1,3]dioxol-5-yl)-1-(4-(diethylcarbamoyl)benzyl)-1H-indazole-3-carboxamide;
N-(benzo[d][1,3]dioxol-5-yl)-1-(4-(isopropylsulfonyl)benzyl)-1H-indazole-3-carboxamide;
N-(benzo[d][1,3]dioxol-5-yl)-1-((5-ethylpyridin-2-yl)methyl)-1H-indazole-3-carboxamide;
N-(benzo[d][1,3]dioxol-5-yl)-1-((5-isopropyl-1,2,4-oxadiazol-3-yl)methyl)-1H-indazole-3-carboxamide;
N-(benzo[d][1,3]dioxol-5-yl)-1-((5-ethyl-1,3,4-oxadiazol-2-yl)methyl)-1H-indazole-3-carboxamide;
N-(benzo[d][1,3]dioxol-5-yl)-1-(4-(ethylsulphonyl)benzyl)-1,4-indazole-3-carboxamide;
1-(4-(methylsulphonyl)benzyl)-1H-indazole-3-carboxylic acid;
N-(2,3-dihydrobenzo[b][1,4]dioxin-6-yl)-1-(4-ethylbenzyl)-1H-indazole-3-carboxamide;
1-(4-ethylbenzyl)-N-(2,2,3,3-tetrafluoro-2,3-dihydrobenzo[b][1,4]dioxin-6-yl)-1H-indazole-3-carboxamide;
N-(2,3-dihydrobenzo[b][1,4]dioxin-6-yl)-5-fluoro-1-(4-(morpholine-4-carbonyl)benzyl)-1H-indazole-3-carboxamide;
1-(4-(morpholine-4-carbonyl)benzyl)-N-(2,2,3,3-tetrafluoro-2,3-dihydrobenzo[b][1,4]dioxin-6-yl)-1H-indazole-3-carboxamide;
N-(2,3-dihydrobenzo[b][1,4]dioxin-6-yl)-6-fluoro-1-(4-(morpholine-4-carbonyl)benzyl)-1H-indazole-3-carboxamide; or
N-(2,3-dihydrobenzo[b][1,4]dioxin-6-yl)-1-(4-(morpholine-4-carbonyl)benzyl)-6-(trifluoromethyl)-1H-indazole-3-carboxamide;
or a tautomer, enantiomer or pharmaceutically acceptable salt, hydrate, solvate, complex or prodrug thereof.

31. A compound, or a tautomer, enantiomer or pharmaceutically acceptable salt, hydrate, solvate, complex or prodrug thereof, wherein the compound is:
1-(4-(Morpholine-4-carbonyl)benzyl)-N-(4-(trifluoromethoxy)phenyl)-1H-indazole-3-carboxamide;
N-(3,4-Dimethoxyphenyl)-1-(4-(methylsulfonyl)benzyl)-1H-indazole-3-carboxamide;
N-(4-Methoxyphenyl)-1-(4-(morpholine-4-carbonyl)benzyl)-1H-indazole-3-carboxamide;
N-(Benzo[d][1,3]dioxol-5-yl)-1-(4-(methylsulfonyl)benzyl)-1H-indole-3-carboxamide; or
N-(Benzo[d][1,3]dioxol-5-yl)-1-(4-(methylsulfonyl)benzyl)-1,4,5,6-tetrahydrocyclopenta[c]pyrazole-3-carboxamide.

32. A pharmaceutical composition comprising the compound of claim 31 and a pharmaceutically acceptable excipient.

33. A method for the therapeutic or prophylactic treatment of Duchenne muscular dystrophy, Becker muscular dystrophy, or cachexia, comprising administering the compound of claim 31.
